(19) United States
(12) Patent Application Publication
(10) Pub. No.: US 2024/0361689 A1
(43) Pub. Date: Oct. 31, 2024

(54) PHOTOSENSITIVE RESIN COMPOSITION, PHOTOSENSITIVE RESIN LAYER USING THE SAME, AND COLOR FILTER

(71) Applicant: SAMSUNG SDI CO., LTD., Yongin-si (KR)

(72) Inventors: Heejo MOON, Suwon-si (KR); Baek Soung PARK, Suwon-si (KR); Juho JUNG, Suwon-si (KR); Chang-Hyun KWON, Suwon-si (KR); Kwangwon SEO, Suwon-si (KR)

(21) Appl. No.: 18/382,720

(22) Filed: Oct. 23, 2023

(30) Foreign Application Priority Data
Apr. 5, 2023 (KR) 10-2023-0044996

(51) Int. Cl. G03F 7/004; C07D 401/14; G03F 7/00; G03F 7/027

(52) U.S. Cl. CPC G03F 7/0048; C07D 401/14; G03F 7/0007; G03F 7/027; G02B 5/223

(57) ABSTRACT

a photosensitive resin composition, a photosensitive resin layer, and a color filter manufactured using the same, the photosensitive resin composition includes a colorant; a photopolymerizable compound; a photopolymerization initiator; a binder resin; and a solvent; wherein the colorant includes a pigment, a dispersant, and a dispersion aid represented by Chemical Formula 1:

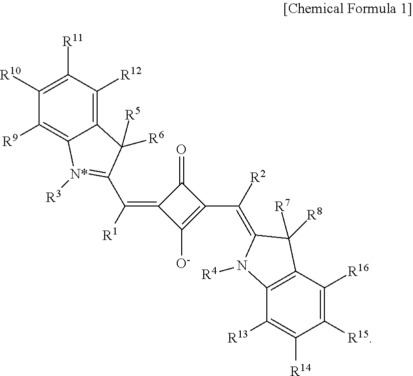

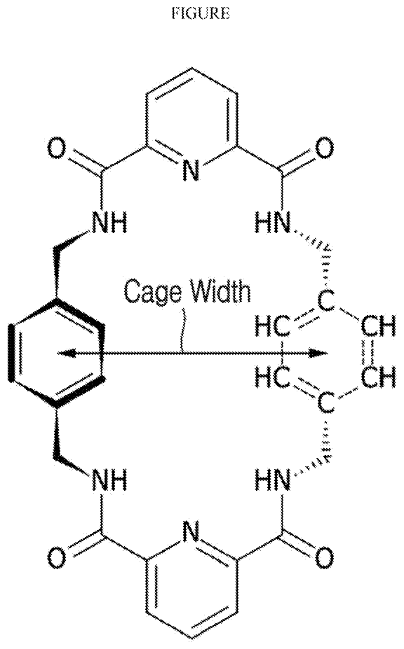

### CROSS-REFERENCE TO RELATED APPLICATION

This application claims priority to and the benefit of Korean Patent Application No. 10-2023-0044996 filed in the Korean Intellectual Property Office on Apr. 5, 2023, the entire contents of which are incorporated herein by reference.

### BACKGROUND

### 1. Field

Embodiments relate to a photosensitive resin composition, a photosensitive resin layer using the same, and a color filter.

### 2. Description of the Related Art

A liquid crystal display device among many types of displays has an advantage of lightness, thinness, low cost, low power consumption for operation, and improved adherence to an integrated circuit and has been used for a laptop computer, a monitor, and a TV screen.

The liquid crystal display device may include a lower substrate on which a black matrix, a color filter, and an ITO pixel electrode are formed, and an upper substrate on which an active circuit portion including a liquid crystal layer, a thin film transistor, and a capacitor layer and an ITO pixel electrode are formed.

Color filters may be formed in a pixel region by sequentially stacking a plurality of color filters (e.g., three primary colors such as red (R), green (G), and blue (B)) in a predetermined order to form each pixel, and a black matrix layer may be disposed in a predetermined pattern on a transparent substrate to form a boundary between the pixels.

The pigment dispersion method, e.g., a method of forming a color filter, may provide a colored thin film by repeating a series of processes such as coating a photopolymerizable composition including a colorant on a transparent substrate including a black matrix, exposing a formed pattern to light, removing a non-exposed part with a solvent, and thermally curing the same.

The photosensitive resin composition (pigment-type photosensitive resin composition) used to manufacture a color filter according to the pigment dispersion method may include an alkali-soluble resin, a photopolymerizable monomer, a photopolymerization initiator, an epoxy resin, a solvent, and other additives. The pigment dispersion method having the above characteristics may used to manufacture LCDs for mobile phones, laptops, monitors, TVs, or the like.

### SUMMARY

The embodiments may be realized by providing a photosensitive resin composition including a colorant; a photopolymerizable compound; a photopolymerization initiator; a binder resin; and a solvent; wherein the colorant includes a pigment, a dispersant, and a dispersion aid represented by Chemical Formula 1:

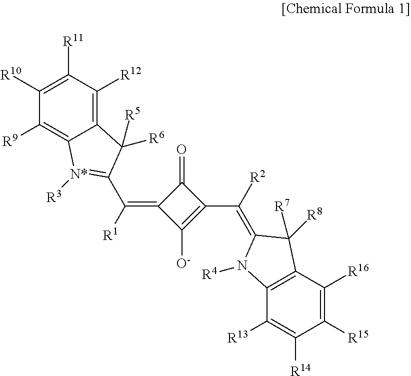

in Chemical Formula 1, R1 to R8 are each independently a hydrogen atom or a substituted or unsubstituted C1 to C20 alkyl group; and R9 to R16 are each independently a hydrogen atom, a substituted or unsubstituted C1 to C20 alkyl group, or a substituted or unsubstituted acidic functional group, provided that at least one of R9 to R16 is an acidic functional group.

The embodiments may be realized by providing a photosensitive resin layer manufactured using the photosensitive resin composition according to an embodiment.

The embodiments may be realized by providing a color filter including the photosensitive resin layer according to an embodiment.

The embodiments may be realized by providing a display device including the color filter according to an embodiment.

### BRIEF DESCRIPTION OF THE DRAWING

Features will be apparent to those of skill in the art by describing in detail exemplary embodiments with reference to the attached drawing in which:

the FIGURE briefly shows the structure of the shell and its width.

### DETAILED DESCRIPTION OF THE EMBODIMENTS

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. As used herein, the term “or” is not necessarily an exclusive term, e.g., “A or B” would include A, B, or A and B.

As used herein, when specific definition is not otherwise provided, “substituted” refers to replacement of at least one hydrogen atom of a compound by a substituent of a halogen atom (F, Cl, Br, or I), a hydroxy group, a C1 to C20 alkoxy group, a nitro group, a cyano group, an amine group, an imino group, an azido group, an amidino group, a hydrazino group, a hydrazono group, a carbonyl group, a carbamyl group, a thiol group, an ester group, an ether group, a carboxyl group or a salt thereof, a sulfonic acid group or a salt thereof, a phosphoric acid or a salt thereof, a C1 to C20 alkyl group, a C2 to C20 alkenyl group, a C2 to C20 alkynyl group, a C6 to C30 aryl group, a C3 to C20 cycloalkyl group, a C3 to C20 cycloalkenyl group, a C3 to C20 cycloalkynyl group, a C2 to C20 heterocycloalkyl group, a C2 to C20 heterocycloalkenyl group, a C2 to C20 heterocycloalkynyl group, or a combination thereof.

As used herein, when specific definition is not otherwise provided, a “heterocycloalkyl group”, a “heterocycloalkenyl group”, a “heterocycloalkynyl group,” and a “heterocycloalkylene group” refer to each cyclic compound of cycloalkyl, cycloalkenyl, cycloalkynyl, and cycloalkylene including at least one heteroatom of N, O, S, or P.

As used herein, when a definition is not otherwise provided, the term “combination” refers to mixing or copolymerization. Additionally, “copolymerization” refers to block copolymerization to random copolymerization, and “copolymer” refers to block copolymerization to random copolymerization.

As used herein, when a definition is not otherwise provided, hydrogen is bonded at the position when a chemical bond is not drawn where supposed to be given.

As used herein, when specific definition is not otherwise provided, “*” refers to a linking point with the same or different atom or chemical formula.

As used herein, when a definition is not otherwise provided, “particle diameter” may mean a diameter of a particle, and the diameter of the particle may be a Z-average value of the particle diameter measured through dynamic light scattering.

### (Photosensitive Resin Composition)

Some example embodiments may provide a photosensitive resin composition including, e.g., (A) a colorant; (B) a photopolymerizable compound; (C) a photopolymerization initiator; (D) a binder resin; and (E) a solvent. In an implementation, the colorant may include, e.g., a pigment, a dispersant, and a dispersion aid represented by Chemical Formula 1.

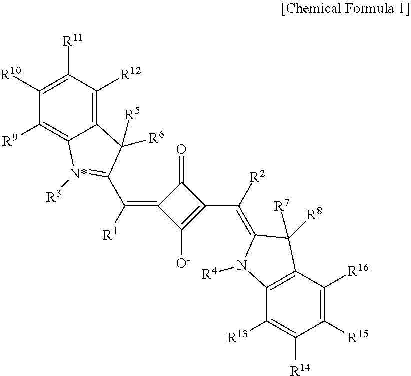

In Chemical Formula 1, R¹ to R⁸ may each independently be or include, e.g., a hydrogen atom or a substituted or unsubstituted C1 to C20 alkyl group.

R⁹ to R¹⁶ may each independently be or include, e.g., a hydrogen atom, a substituted or unsubstituted C1 to C20 alkyl group, or a substituted or unsubstituted acidic functional group. In an implementation, at least one of R⁹ to R¹⁶ may be, e.g., a substituted or unsubstituted acidic functional group.

### (A) Colorant

The photosensitive resin composition of some example embodiments may be a pigment-type photosensitive resin composition, and the colorant may include a pigment. A color filter manufactured by using a pigment-type photosensitive resin composition could have a limit in terms of luminance caused by a pigment particle size. Additionally, in order to be applied to image sensors, a resin composition made of smaller particles may be required to form fine patterns. In order to achieve this, a compound and a composition using the same may help disperse the pigment and prevent re-agglomeration.

In this regard, the colorant may include the dispersion aid represented by Chemical Formula 1.

The dispersion aid represented by Chemical Formula 1 may function as a dispersion aid and a colorant.

The dispersion aid represented by Chemical Formula 1 may have a mother moiety in which both benzene rings of squarene (SQ) are each substituted with an indole ring, and at least one of the indole rings may include an acidic functional group and may interact with the pigment, the dispersant, or both.

In an implementation, in the dispersion aid represented by Chemical Formula 1, the acidic functional group may be covalently bonded with the dispersant or dispersion resin, and the mother moiety may be covalently bonded with the pigment, which may help the interaction between the pigment and the dispersant. As a result, it is possible to provide a photosensitive resin composition that has high dispersibility and dispersion stability and has excellent coloring power and luminance even when the color filter is thinned.

In the dispersion aid represented by Chemical Formula 1, the mother moiety may express green color, so that the coloring power and luminance of the color filter may be further improved.

By using the photosensitive resin composition according to some example embodiments including this, a color filter and display device with excellent color reproducibility and luminance may be implemented.

R¹ to R⁸ may each independently be, e.g., a hydrogen atom or a substituted or unsubstituted C1 to C20 alkyl group.

In an implementation, both R¹ and R² may be hydrogen atoms.

In an implementation, both R³ and R⁴ may be hydrogen atoms.

In an implementation, R⁵ to R⁸ may each independently be, e.g., a substituted or unsubstituted C1 to C20 alkyl group. In an implementation, all of R⁵ to R⁸ may be a substituted or unsubstituted methyl group.

In an implementation, one of R⁹ to R¹² may be a substituted or unsubstituted acidic functional group and one of R¹³ and R¹⁶ be a substituted or unsubstituted acidic functional group. In an implementation, R¹¹ and R¹⁵ may each independently be a substituted or unsubstituted acidic functional group. In an implementation, the dispersion aid may be represented by, e.g., Chemical Formula 1-1.

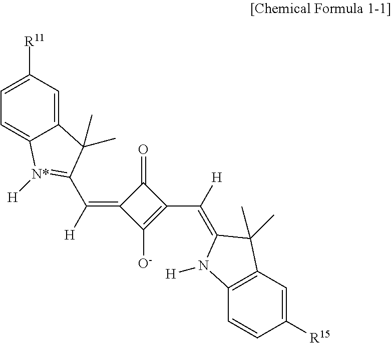

In Chemical Formula 1-1, R¹¹ and R¹⁵ may each independently be, e.g., a substituted or unsubstituted acidic functional group.

In an implementation, the substituted or unsubstituted acidic functional group may be an amine group having a terminal end substituted with *—COOH or *—SO₃H, e.g., an amine group having *—COOH or *—SO₃H at a terminal end thereof. In an implementation, the substituted or unsubstituted acidic functional group may be represented by Chemical Formula A.

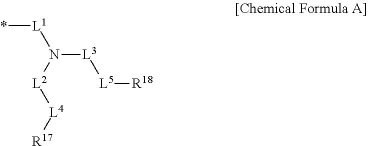

In Chemical Formula A, L1 may be or may include, e.g., a single bond or a substituted or unsubstituted C1 to C20 alkylene group.

L² and L³ may each independently be or include, e.g., a single bond, a substituted or unsubstituted C1 to C20 alkylene group, or *—(C—O)—*.

L⁴ and L⁵ may each independently be or include, e.g., a single bond or a substituted or unsubstituted C1 to C20 alkylene group.

R¹⁷ and R¹⁸ may each independently be or include, e.g., a hydrogen atom, *—COOH, or *—SO₃H. In an implementation, at least one of R¹⁷ and R¹⁸ may be, e.g., *—COOH, or *—SO₃H.

In an implementation, L1 may be, e.g., a substituted or unsubstituted C1 to C20 alkylene group. In an implementation, L1 may be, e.g., a substituted or unsubstituted methylene group.

In an implementation, L2 and L3 may both be single bonds, or both may be *—(C═O)—*.

In an implementation, L4 and L5 may both be, e.g., a substituted or unsubstituted ethylene group or a substituted or unsubstituted propylene group.

In an implementation, R¹⁷ and R¹⁸ may each independently be, e.g., *—COOH or *—SO₃H.

In an implementation, the substituted or unsubstituted acidic functional group may be one of the following groups.

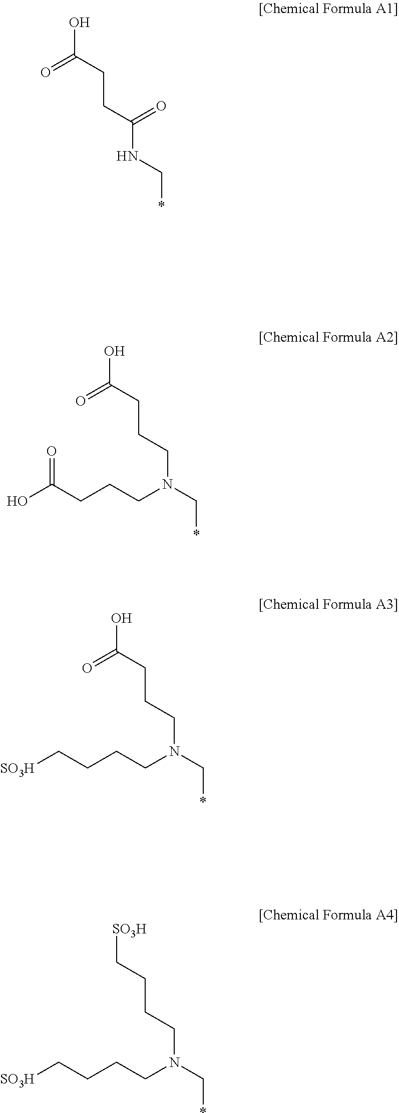 [SMILES: *CNC(=O)CCC(=O)O ; *CN(CCCC(=O)O)CCCC(=O)O ; *CN(CCCCS(=O)(=O)O)CCCC(=O)O ; *CN(CCCCS(=O)(=O)O)CCCCS(=O)(=O)O]

In an implementation, the dispersion aid may have a symmetric structure or an asymmetric structure. In an implementation, a symmetric structure may be advantageous in that the synthesis step may be short and easy, thereby lowering the manufacturing cost. In an implementation, the acidic functional group may be represented by, e.g., Chemical Formula A2 or A4.

In an implementation, the dispersion aid may include a compound represented by one of the following Chemical Formulae.

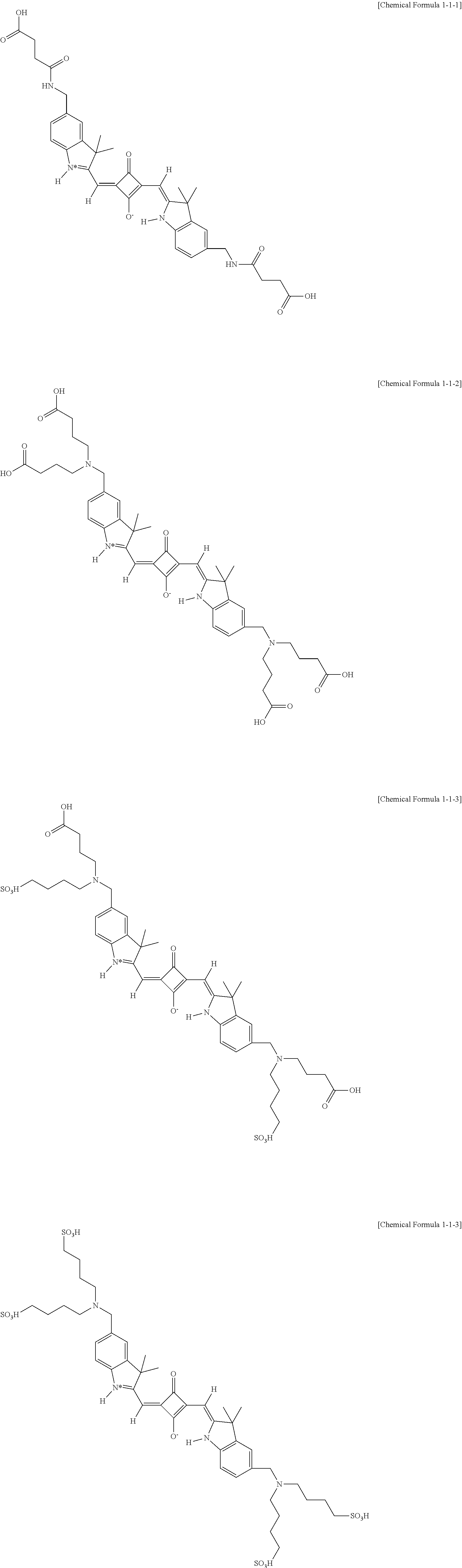

In an implementation, the dispersion aid may include the compound represented by Chemical Formula 1 as a core, and may further include a shell surrounding the core. In an implementation, the shell may be represented by, e.g., Chemical Formula 2.

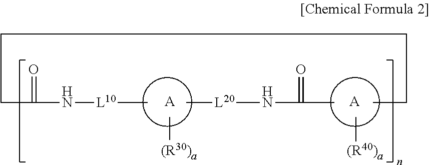

In Chemical Formula 2, A and B may each independently be or include, e.g., a benzene ring, a pyridine ring, or a furan ring.

L¹⁰ and L²⁰ may each independently be or include, e.g., a single bond or a substituted or unsubstituted C1 to C10 alkylene group.

R³⁰ and R⁴⁰ may each independently be, e.g., a halogen.

a and b may each independently be, e.g., an integer of 0 to 4.

n may be, e.g., an integer of 2 to 10.

In an implementation, A may be a benzene ring, and B may be a benzene ring, a pyridine ring, or a furan ring.

In an implementation, L¹⁰ and L²⁰ may each independently be, e.g., a substituted or unsubstituted C1 to C10 alkylene group. In an implementation, both L¹⁰ and L²⁰ may be, e.g., a substituted or unsubstituted methylene group.

In an implementation, n may be 2.

In an implementation, the shell may be represented by one of the following Chemical Formulae.

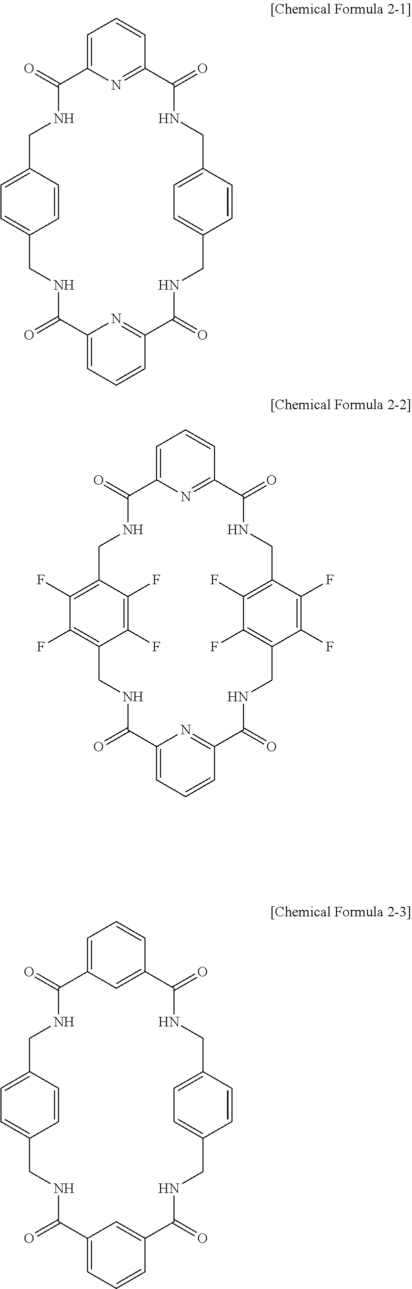 [SMILES: O=C1NCc2ccc(cc2)CNC(=O)c2cccc(n2)C(=O)NCc2ccc(cc2)CNC(=O)c2cccc1n2 ; O=C1NCc2c(F)c(F)c(c(F)c2F)CNC(=O)c2cccc(n2)C(=O)NCc2c(F)c(F)c(c(F)c2F)CNC(=O)c2cccc1n2 ; O=C1NCc2ccc(cc2)CNC(=O)c2cccc(c2)C(=O)NCc2ccc(cc2)CNC(=O)c2cccc1c2]

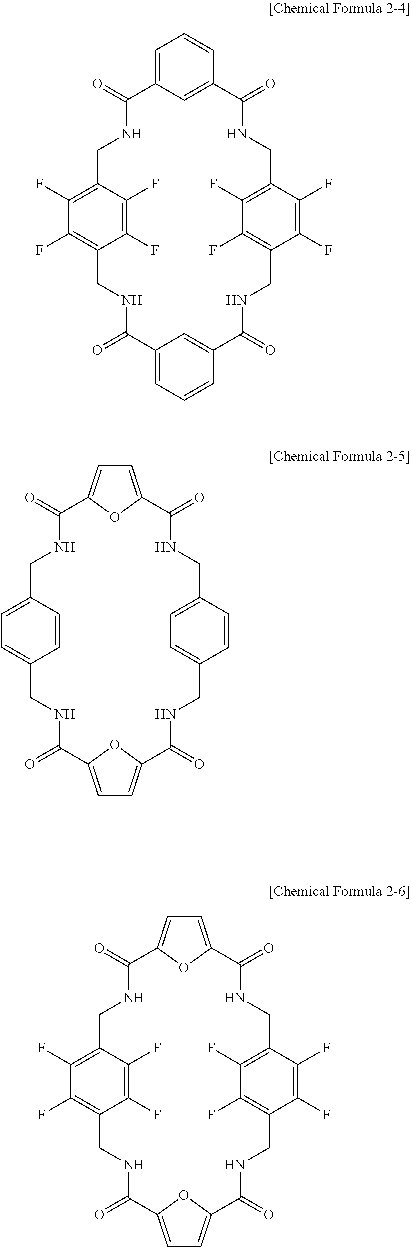 [SMILES: O=C1NCc2c(F)c(F)c(c(F)c2F)CNC(=O)c2cccc(c2)C(=O)NCc2c(F)c(F)c(c(F)c2F)CNC(=O)c2cccc1c2 ; O=C1NCc2ccc(cc2)CNC(=O)c2ccc(o2)C(=O)NCc2ccc(cc2)CNC(=O)c2ccc1o2 ; O=C1NCc2c(F)c(F)c(c(F)c2F)CNC(=O)c2ccc(o2)C(=O)NCc2c(F)c(F)c(c(F)c2F)CNC(=O)c2ccc1o2]

In an implementation, the dispersion aid having the core-shell structure may be represented by one of the following Chemical Formulae.

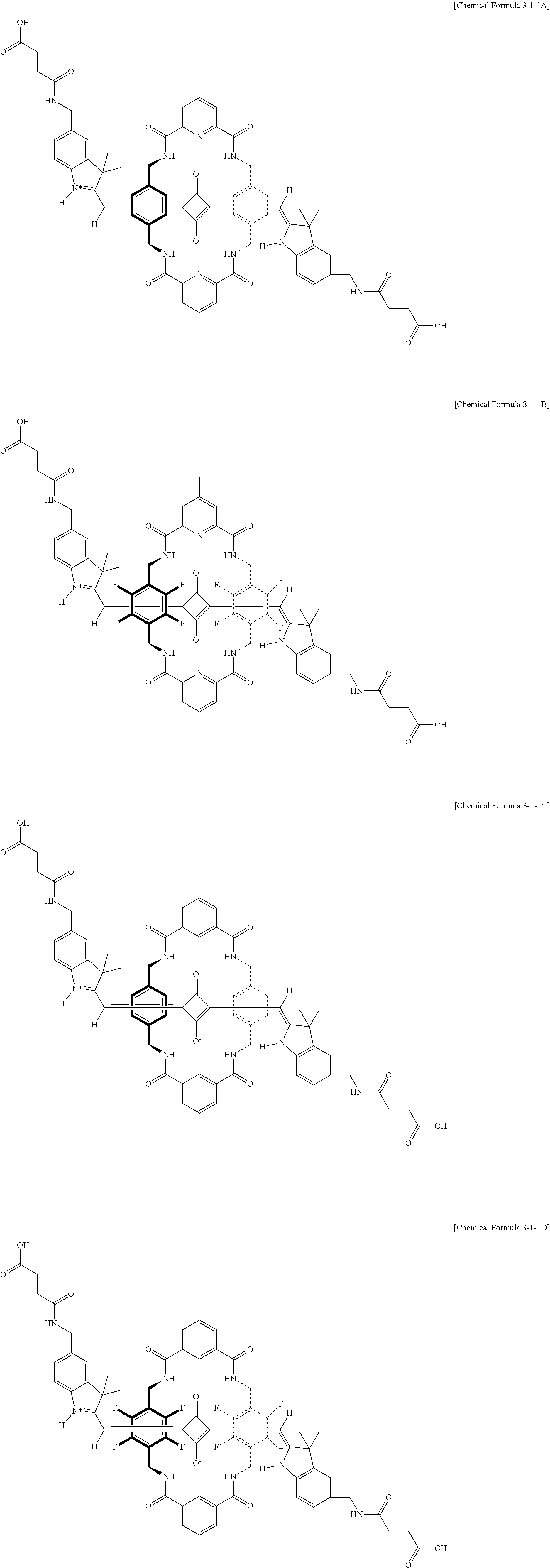

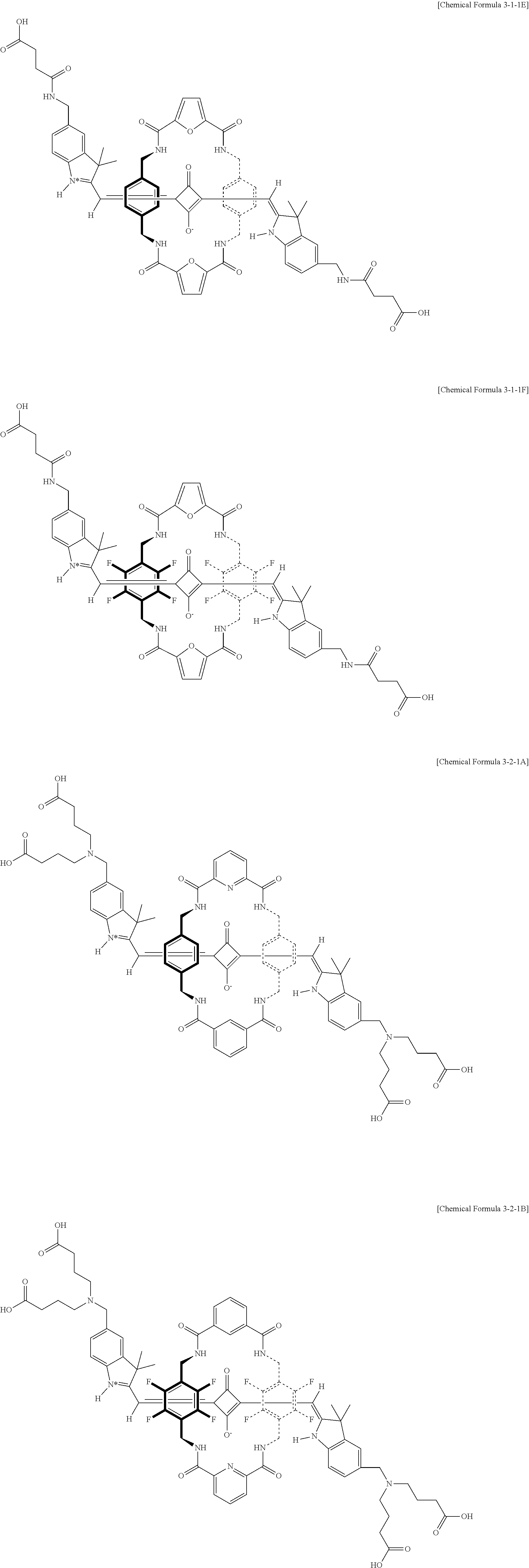

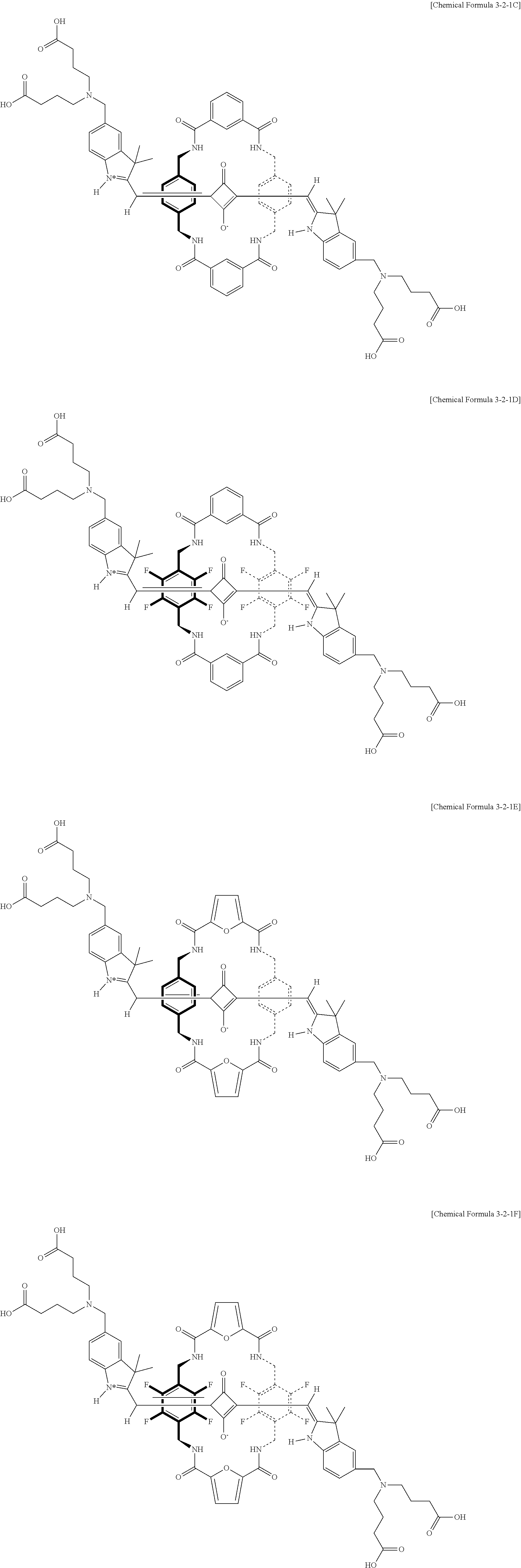

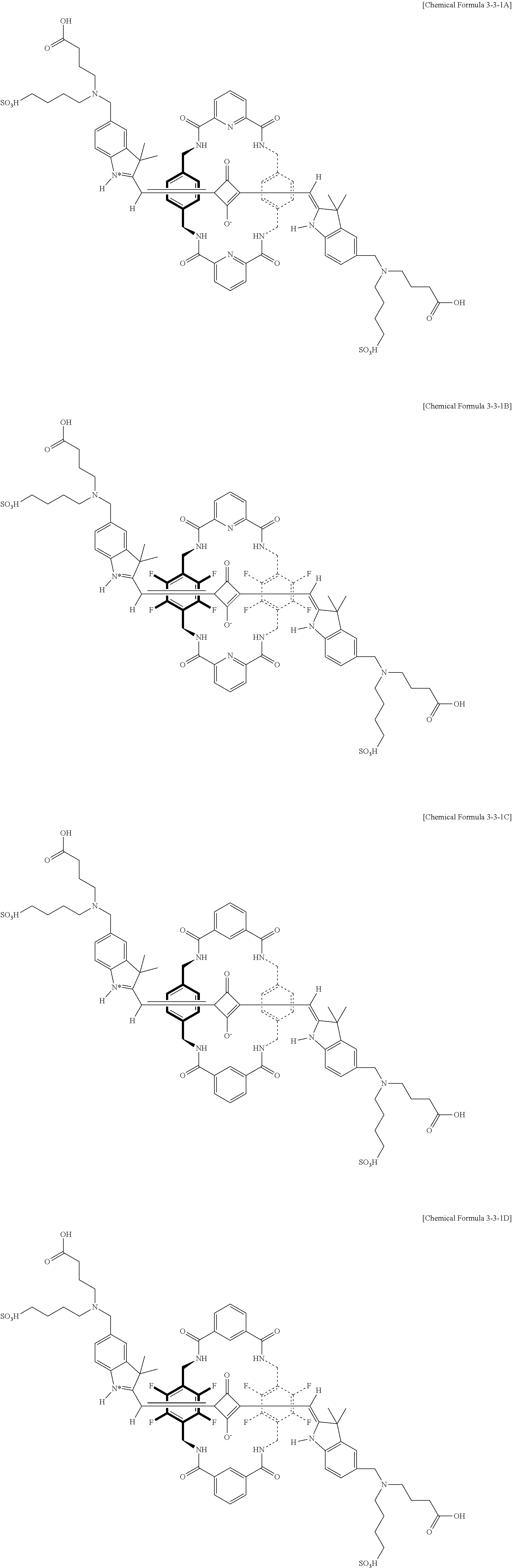

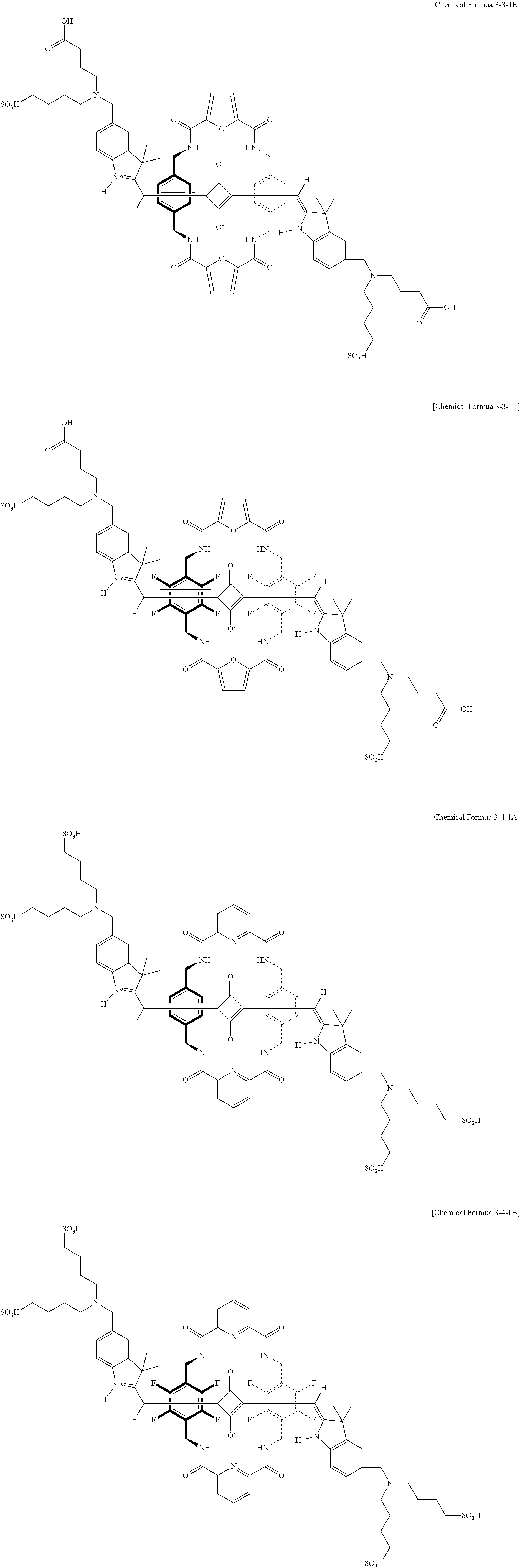

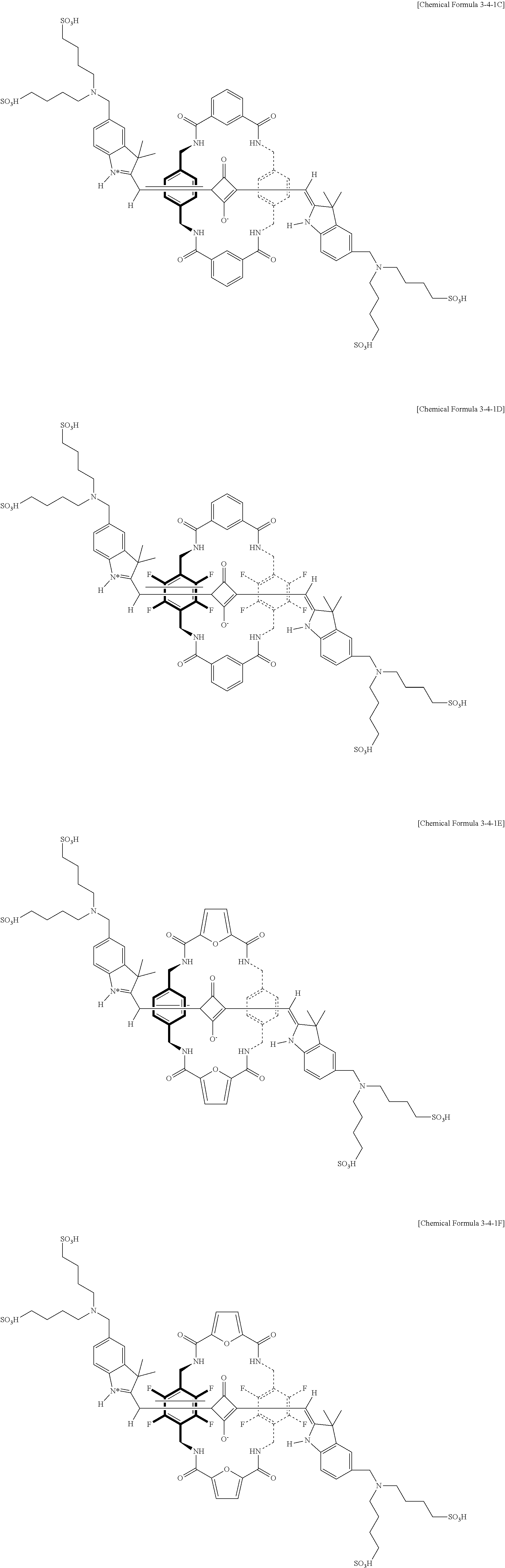

A length of the core represented by Chemical Formula 1 may be about 1 nm to about 3 nm, e.g., about 1.5 nm to about 2 nm. Within the above ranges, a core-shell structured compound may be easily formed. Within the above ranges, the shell of the macrocyclic compound may surround the core represented by Chemical Formula 1. If a different core that does not fall within the above ranges were to be used, it could be difficult to expect improvement in durability because it could be difficult for the shell to form a structure surrounding the core.

The cage width in this disclosure refers to an internal distance of the shell, e.g., in a shell represented by Chemical Formula 2, e.g., a distance between two different phenylene groups in which both methylene groups are linked (refer to the FIGURE).

The wider the cage width of the shell, the more likely slip-off of the cage could occur, and the higher the possibility that the shell could come out of the dye, which in turn could reduce the durability of the dye. If the cage width of the shell were to be too small, synthesis itself could be impossible. Accordingly, the cage size of the shell may be appropriately set.

A cage width of the shell may range from about 6.5 Å to about 7.5 Å and a volume of the shell may range from about 10 Å to about 16 Å. Within the ranges, a core-shell dye having a structure surrounding the core represented by Chemical Formula 1 may be obtained, and thus a color filter having improved durability may be realized when the core-shell dye is added to a photosensitive resin composition.

The shell represented by Chemical Formula 2 may include an amide bond (—CONH—). The hydrogen atom included in the amide bond of the shell represented by Chemical Formula 2 may form a non-covalent bond with the oxygen atom of the compound represented by Chemical Formula 1. In an implementation, the two atoms may form a hydrogen bond, which may help enhance the durability of the core-shell dye.

In an implementation, the dispersion aid may have a squarene-based mother moiety that expresses green color, and may help further improve the green coloring power and luminance of the photosensitive resin composition.

In an implementation, the dispersion aid may have a maximum absorption wavelength (λₘₐₓ) of about 530 to about 700 nm.

In an implementation, the core itself represented by Chemical Formula 1 may have a maximum absorption peak at a wavelength of about 530 nm to about 680 nm. In an implementation, the maximum absorption peak of the core-shell dye may vary depending on whether a halogen group is introduced into the shell represented by Chemical Formula 2. In an implementation, the halogen group may be introduced into the shell represented by Chemical Formula 2, and the dispersion aid may have a maximum absorption peak at a wavelength of about 530 nm to about 700 nm, e.g., about 550 to about 700 nm.

In an implementation, the halogen group may be introduced into the shell represented by Chemical Formula 2, the maximum absorption peak of the core-shell compound may shift to a long wavelength region of about 20 nm, and excellent matching to the green wavelength band may be achieved.

In an implementation, the dispersion aid may be a compound of a core-shell structure, and it may include a core containing the compound represented by Chemical Formula 1 and the shell at a molar ratio of about 1:1. Within the above molar ratio, a coating layer (shell) surrounding the core containing the compound represented by Chemical Formula 1 may be well formed.

The dispersion aid may be included in an amount of about 0.5 wt % to about 6 wt %, e.g., about 1 wt % to about 5 wt %, or about 2 wt % to about 3 wt %, based on a total weight of a photosensitive resin composition.

In an implementation, a weight ratio of the dispersion aid and the pigment may be about 1:20 to about 1:70, e.g., about 1:30 to about 1:60, or about 1:40 to about 1:50. In an implementation, the dispersion aid and the pigment may each be solid.

Within the above ranges, the dispersibility and dispersion stability of the photosensitive resin composition according to the embodiment may be improved while the coloring power and luminance of the color filter may be improved.

The colorant may include a pigment, and the pigment may include a green pigment, a blue pigment, a red pigment, a violet pigment, a yellow pigment, or a black pigment.

The red pigment may include, e.g., C.I. Red Pigment 254, C.I. Red Pigment 255, C.I. Red Pigment 264, C.I. Red Pigment 270, C.I. Red Pigment 272, C.I. Red Pigment 177, C.I. Red Pigment 89, or the like in the color index, which may be used alone or in a mixture of two or more.

The violet pigment may include, e.g., C.I. Violet Pigment 23 (V.23), C.I. Violet Pigment 29, Dioxazine Violet, First Violet B, Methyl Violet Lake, Indanethrene Brilliant Violet, or the like in the color index, which may be used alone or in a mixture of two or more.

The green pigment may include, e.g., C.I. Green Pigment 7, C.I. Green Pigment 36, C.I. Green Pigment 58, C.I. Green Pigment 59 or the like in the color index, which may be used alone or in a mixture of two or more.

The blue pigment may include, e.g., copper phthalocyanine pigments such as C.I. Blue Pigment 15:6, C.I. Blue Pigment 15, C.I. Blue Pigment 15:1, C.I. Blue Pigment 15:2, C.I. Blue Pigment 15:3, C.I. Blue Pigment 15:4, C.I. Blue Pigment 15:5, C.I. Blue Pigment 15:6, C.I. Blue Pigment 16 or the like in the color index, which may be used alone or in a mixture of two or more.

The yellow pigment may include, e.g., an isoindoline pigment such as C.I. Yellow Pigment 185, C.I. Yellow Pigment 139, or the like, a quinophthalone pigment such as C.I. Yellow Pigment 138, a nickel complex pigment such as C.I. Yellow Pigment 150 or the like in the color index, which may be used alone or in a mixture of two or more.

The black pigment may include, e.g., aniline black, perylene black, titanium black, carbon black, or the like in the color index, which may be used alone or in a mixture of two or more.

The pigments may be used alone or in a mixture of two or more. In an implementation, the pigment may be a green pigment, a yellow pigment, or a mixture thereof.

The dispersant may help the pigment to be uniformly dispersed in the dispersion, and nonionic, anionic or cationic dispersants may be used, respectively. In an implementation, polyalkylene glycol or its ester, polyoxy alkylene, a polyhydric alcohol ester alkylene oxide adduct, an alcohol alkylene oxide adduct, sulfonic acid ester, sulfonic acid salt, carboxylic acid ester, carboxylic acid salt, alkyl amide alkylene oxide adduct, an alkylamine, or the like may be used, and these may be used alone or in combination of two or more.

The pigment may be included in the photosensitive resin composition for a color filter in the form of a dispersion. This pigment dispersion may further include a dispersion solvent, a dispersion resin, or the like, in addition to the pigment, the dispersant, and the dispersion aid. Solid pigment excluding the solvent may be included in an amount of about 5 wt % to about 20 wt %, e.g., about 8 wt % to about 15 wt %, based on a total weight of the pigment dispersion.

Solvents for the pigment dispersion may include, e.g., ethylene glycol acetate, ethyl cellosolve, propylene glycol methyl ether acetate, ethyl lactate, polyethylene glycol, cyclohexanone, propylene glycol methyl ether, or the like. In an implementation, the solvent may include, e.g., propylene glycol methyl ether acetate.

The dispersion resin may be an acrylic resin containing a carboxy group may be used, which may help improve stability of the pigment dispersion and also improve pixel patternability.

The colorant may include the pigment and may further include a dye, and in this case, the resin composition of some example embodiments may be a hybrid type composition. In an implementation, the dye may include a metal complex dye.

The metal complex dye may be a compound having maximum absorbance in the wavelength range of 200 nm to 650 nm, and if the compound has absorbance in the above range in order to match the color coordinates to the combination of dyes, the metal complex dye of all colors that dissolves in an organic solvent may be used.

In an implementation, the metal complex dye may be a green dye having maximum absorbance in a wavelength range of 530 nm to 680 nm, a yellow dye having maximum absorbance in a wavelength range of 200 nm to 400 nm, an orange dye having a maximum absorbance in a wavelength range of 300 nm to 500 nm, a red dye having maximum absorbance in a wavelength range of 500 nm to 650 nm, or a combination thereof.

The metal complex dye may be, e.g., a direct dye, an acidic dye, a basic dye, an acidic mordant dye, a sulfide dye, a reduction dye, an azoic dye, a dispersion dye, a reactive dye, an oxidation dye, an oil-soluble dye, an azo dye, an anthraquinone dye, an indigoid dye, a carbonium ion dye, a phthalocyanine dye, a nitro dye, a quinoline dye, a cyanine dye, a polymethine dye, or a combination thereof.

The metal complex dye may include a metal ion of, e.g., Mg, Ni, Cu, Co, Zn, Cr, Pt, Pd, or Fe.

The metal complex dye may be a complex of, e.g., C.I. Solvent Dye such as C.I. Solvent Green 1, 3, 4, 5, 7, 28, 29, 32, 33, 34, 35, or the like; C.I. Acid Dye such as C.I. Acid Green 1, 3, 5, 6, 7, 8, 9, 11, 13, 14, 15, 16, 22, 25, 27, 28, 41, 50, 50:1, 58, 63, 65, 80, 104, 105, 106, 109, or the like; C.I. Direct Dye such as C.I. Direct Green 25, 27, 31, 32, 34, 37, 63, 65, 66, 67, 68, 69, 72, 77, 79, 82, or the like; C.I. Basic Dye such as C.I. Basic Green 1, or the like; C.I. Mordant Dye such as C.I. Mordant Green 1, 3, 4, 5, 10, 13, 15, 19, 21, 23, 26, 29, 31, 33, 34, 35, 41, 43, 53, or the like; C.I. Green pigments such as Pigment Green 7, 36, 58, or the like; Solvent Yellow 19, Solvent Yellow 21, Solvent Yellow 25, Solvent Yellow 79, Solvent Yellow 82, Solvent Yellow 88, Solvent Orange 45, Solvent Orange 54, Solvent Orange 62, Solvent Orange 99, Solvent Red 8, Solvent Red 32, Solvent Red 109, Solvent Red 112, Solvent Red 119, Solvent Red 124, Solvent Red 160, Solvent Red 132, or Solvent Red 218, and the metal ion.

The metal complex dye may have a solubility of greater than or equal to about 5, e.g., about 5 to about 10, in a solvent used in the photosensitive resin composition according to some embodiments. The solubility may be obtained by an amount (g) of the dye dissolved in 100 g of the solvent. Within the above ranges, compatibility with other components constituting the photosensitive resin composition according to some embodiments and coloring power may be secured, and precipitation of the dye may be prevented.

The solvent may include, e.g., propylene glycol monomethyl ether acetate (PGMEA), ethyl lactate (EL), ethylene glycol ethyl acetate (EGA), cyclohexanone, 3-methoxy-1-butanol, or a combination thereof.

In the above ranges, it can be usefully used for color filters such as LCDs and LEDs that express high luminance and high contrast ratio in a desired color coordinate.

The metal complex dye may be included in an amount of about 0.01 wt % to about 1 wt %, e.g., about 0.01 wt % to about 0.5 wt %, based on a total weight of the photosensitive resin composition. Within the above ranges, high luminance and contrast ratio can be exhibited in a desired color coordinate.

In an implementation, the dye and the pigment may be mixed and used, e.g., in a weight ratio of about 0.1:99.9 to about 99.9:0.1, or about 1:9 to about 9:1. Within the above weight ratio ranges, chemical resistance and maximum absorption wavelength may be controlled within an appropriate range, and high luminance and contrast ratio may be exhibited in a desired color coordinate.

The colorant may be included in an amount of about 5 wt % to about 50 wt %, e.g., about 6 wt % to about 40 wt %, or about 7 wt % to about 30 wt %, based on a total solid content of the photosensitive resin composition. Within the above ranges, a coloring effect and developability are improved.

### (B) Photopolymerizable Compound

The photopolymerizable compound may include, e.g., a mono-functional or multi-functional ester of (meth)acrylic acid including at least one ethylenic unsaturated double bond.

The photopolymerizable compound may cause sufficient polymerization during exposure in a pattern-forming process and form a pattern having excellent heat resistance, light resistance, and chemical resistance due to the ethylenic unsaturated double bond.

Examples of the photopolymerizable compound may include ethylene glycol di(meth)acrylate, diethylene glycol di(meth)acrylate, triethylene glycol di(meth)acrylate, propylene glycol di(meth)acrylate, neopentyl glycol di(meth)acrylate, 1,4-butanediol di(meth)acrylate, 1,6-hexanediol di(meth)acrylate, bisphenol A di(meth)acrylate, pentaerythritol di(meth)acrylate, pentaerythritol tri (meth)acrylate, pentaerythritol tetra(meth)acrylate, pentaerythritol hexa (meth)acrylate, dipentaerythritol di(meth)acrylate, dipentaerythritol tri (meth)acrylate, dipentaerythritol penta (meth)acrylate, dipentaerythritol hexa (meth)acrylate, bisphenol A epoxy (meth)acrylate, ethylene glycol monomethylether (meth)acrylate, trimethylol propane tri (meth)acrylate, tris(meth)acryloyloxyethyl phosphate, novolacepoxy (meth)acrylate, and the like.

Commercially available examples of the photopolymerizable compound may be as follows. The mono-functional (meth)acrylic acid ester may include Aronix M-101®, Aronix M-111R, Aronix M-114® (Toagosei Chemistry Industry Co., Ltd.); KAYARAD TC-110SR, KAYARAD TC-120SR (Nippon Kayaku Co., Ltd.); V-158R, V-2311R (Osaka Organic Chemical Ind., Ltd.), and the like. Examples of a difunctional (meth)acrylic acid ester may include Aronix M-210R, Aronix M-240R, Aronix M-6200R (Toagosei Chemistry Industry Co., Ltd.), KAYARAD HDDAR, KAYARAD HX-220R, KAYARAD R-604R (Nippon Kayaku Co., Ltd.), V-260R, V-312R, V-335 HPR (Osaka Organic Chemical Ind., Ltd.), and the like. Examples of a tri-functional (meth)acrylic acid ester may include Aronix M-309R, Aronix M-400R, Aronix M-4058, Aronix M-450R, Aronix M-7100R, Aronix M-8030R, Aronix M-8060R (Toagosei Chemistry Industry Co., Ltd.); KAYARAD TMPTAR, KAYARAD DPCA-20R, KAYARAD DPCA-30R, KAYARAD DPCA-60R, KAYARAD DPCA-120R (Nippon Kayaku Co., Ltd.); V-295R, V-300R, V-360R, V-GPTR, V-3PAR, V-400R (Osaka Yuki Kayaku Kogyo Co. Ltd.), and the like. These may be used alone or as a mixture of two or more.

The photopolymerizable compound may be used by treating it with an acid anhydride to provide better developability.

The photopolymerizable compound may be included in an amount of about 0.1 wt % to about 20 wt %, e.g., about 1 wt % to about 15 wt %, or about 2 wt % to about 13 wt %, based on a total weight of the photosensitive resin composition. Within the above ranges, sufficient curing may occur during exposure to light in the pattern formation process, resulting in excellent reliability and excellent developability in an alkaline developer.

### (C) Photopolymerization Initiator

The photopolymerization initiator may be an initiator suitably used in photosensitive resin compositions, e.g., an acetophenone compound, a benzophenone compound, a thioxanthone compound, a benzoin compound, a triazine compound, an oxime compound, or a combination thereof.

Examples of the acetophenone compound may include 2,2′-diethoxy acetophenone, 2,2′-dibutoxy acetophenone, 2-hydroxy-2-methylpropinophenone, p-t-butyltrichloro acetophenone, p-t-butyldichloro acetophenone, 4-chloro acetophenone, 2,2′-dichloro-4-phenoxy acetophenone, 2-methyl-1-(4-(methylthio)phenyl)-2-morpholinopropan-1-one, 2-benzyl-2-dimethylamino-1-(4-morpholinophenyl)-butan-1-one, and the like.

Examples of the benzophenone compound may include benzophenone, benzoyl benzoate, benzoyl methyl benzoate, 4-phenyl benzophenone, hydroxy benzophenone, acrylated benzophenone, 4,4′-bis(dimethyl amino)benzophenone, 4,4′-bis(diethylamino) benzophenone, 4,4′-dimethylaminobenzophenone, 4,4′-dichlorobenzophenone, 3,3′-dimethyl-2-methoxybenzophenone, and the like.

Examples of the thioxanthone compound may include thioxanthone, 2-methylthioxanthone, isopropyl thioxanthone, 2,4-diethyl thioxanthone, 2,4-diisopropyl thioxanthone, 2-chlorothioxanthone, and the like.

Examples of the benzoin compound may include benzoin, benzoin methyl ether, benzoin ethyl ether, benzoin isopropyl ether, benzoin isobutyl ether, benzyldimethylketal, and the like.

Examples of the triazine compound may include 2,4,6-trichloro-s-triazine, 2-phenyl 4,6-bis(trichloromethyl)-s-triazine, 2-(3′, 4′-dimethoxystyryl)-4,6-bis(trichloromethyl)-s-triazine, 2-(4′-methoxynaphthyl)-4,6-bis(trichloromethyl)-s-triazine, 2-(p-methoxyphenyl)-4,6-bis(trichloromethyl)-s-triazine, 2-(p-tolyl)-4,6-bis(trichloro methyl)-s-triazine, 2-biphenyl 4,6-bis(trichloro methyl)-s-triazine, bis(trichloromethyl)-6-styryl-s-triazine, 2-(naphthol-yl)-4,6-bis(trichloromethyl)-s-triazine, 2-(4-methoxynaphthol-yl)-4,6-bis(trichloromethyl)-s-triazine, 2-4-bis(trichloromethyl)-6-piperonyl-s-triazine, 2-4-bis(trichloromethyl)-6-(4-methoxystyryl)-s-triazine, and the like.

Examples of the oxime compound may include an O-acyloxime-based compound, 2-(o-benzoyloxime)-1-[4-(phenylthio)phenyl]-1,2-octanedione, 1-(o-acetyloxime)-1-[9-ethyl-6-(2-methylbenzoyl)-9H-carbazol-3-yl]ethanone, O-ethoxycarbonyl-α-oxyamino-1-phenylpropan-1-one, and the like. Specific examples of the O-acyloxime-based compound may be 1,2-octanedione, 2-dimethylamino-2-(4-methylbenzyl)-1-(4-morpholin-4-yl-phenyl)-butan-1-one, 1-(4-phenylsulfanyl phenyl)-butane-1,2-dione2-oxime-O-benzoate, 1-(4-phenylsulfanyl phenyl)-octane-1,2-dione2-oxime-O-benzoate, 1-(4-phenylsulfanyl phenyl)-octan-1-oneoxime-O-acetate and 1-(4-phenylsulfanyl phenyl)-butan-1-oneoxime-O-acetate, and the like.

In an implementation, the photopolymerization initiator may include a carbazole compound, a diketone compound, a sulfonium borate compound, a diazo compound, an imidazole compound, a biimidazole compound, or a fluorene-based compound.

The photopolymerization initiator may be used with a photosensitizer capable of causing a chemical reaction by absorbing light and becoming excited and then, transferring its energy.

Examples of the photosensitizer may include tetraethylene glycol bis-3-mercapto propionate, pentaerythritol tetrakis-3-mercapto propionate, dipentaerythritol tetrakis-3-mercapto propionate, and the like.

The photopolymerization initiator may be included in an amount of about 0.1 wt % to about 5 wt %, e.g., about 1 wt % to about 3 wt %, based on a total weight of the photosensitive resin composition. Within the ranges, sufficient photopolymerization may occur during exposure in a pattern-forming process, excellent reliability may be realized, heat resistance, light resistance, and chemical resistance of patterns, resolution and close contacting properties may be improved, and decrease of transmittance due to a non-reaction initiator may be prevented.

### (D) Binder Resin

The binder resin may include, e.g., an acrylic binder resin.

The acrylic resin may be a copolymer of a first ethylenic unsaturated monomer and a second ethylenic unsaturated monomer that is copolymerizable therewith, and may be a resin including at least one acryl repeating unit.

The first ethylenic unsaturated monomer may be an ethylenic unsaturated monomer including at least one carboxyl group and examples of the monomer may include acrylic acid, methacrylic acid, maleic acid, itaconic acid, fumaric acid, or a combination thereof.

The first ethylenic unsaturated monomer may be included in an amount of about 5 wt % to about 50 wt %, e.g., about 10 wt % to about 40 wt %, based on a total weight of the acrylic binder resin.

The second ethylenic unsaturated monomer may be an aromatic vinyl compound such as styrene, α-methylstyrene, vinyl toluene, vinylbenzylmethylether or the like; an unsaturated carboxylate ester compound such as methyl (meth)acrylate, ethyl (meth)acrylate, butyl (meth)acrylate, 2-hydroxyethyl (meth)acrylate, 2-hydroxy butyl (meth)acrylate, benzyl (meth)acrylate, cyclohexyl (meth)acrylate, phenyl (meth)acrylate, or the like; an unsaturated amino alkyl carboxylate ester compound such as 2-aminoethyl (meth)acrylate, 2-dimethylaminoethyl (meth)acrylate, or the like; a carboxylic acid vinyl ester compound such as vinyl acetate, vinyl benzoate, or the like; an unsaturated glycidyl carboxylate ester compound such as glycidyl (meth)acrylate, or the like; a vinyl cyanide compound such as (meth)acrylonitrile or the like; an unsaturated amide compound such as (meth) acrylamide, or the like; or the like, and may be used alone or as a mixture of two or more.

Examples of the acrylic resin may include a (meth)acrylic acid/benzylmethacrylate copolymer, a (meth)acrylic acid/benzylmethacrylate copolymer, a (meth)acrylic acid/benzylmethacrylate/styrene copolymer, a (meth)acrylic acid/benzylmethacrylate/2-hydroxyethylmethacrylate copolymer, a (meth)acrylic acid/benzylmethacrylate/styrene/2-hydroxyethylmethacrylate copolymer, and the like, and these may be used alone or as a mixture of two or more.

The binder resin may include an epoxy binder resin.

The binder resin may help improve heat resistance by further including an epoxy binder resin. The epoxy binder resin may include, e.g., a phenol novolac epoxy resin, a tetramethyl biphenyl epoxy resin, a bisphenol A epoxy resin, a bisphenol F epoxy resin, an alicyclic epoxy resin, or a combination thereof.

In an implementation, the binder resin including the epoxy binder resin may help secure dispersion stability of a colorant such as a pigment, and may help form a pixel having a desired resolution during a developing process.

The epoxy binder resin may be included in an amount of about 1 wt % to about 10 wt %, e.g., about 5 wt % to about 10 wt %, based on a total weight of the binder resin. Within the above ranges, film residue ratio and chemical resistance may be greatly improved.

An epoxy equivalent weight of the epoxy-based resin may be about 150 g/eq to about 200 g/eq. Within the above range, there may be an advantageous effect in improving a curing degree of the formed pattern and fixing the colorant in the structure in which the pattern is formed.

The binder resin may be dissolved in a solvent in a solid form to form a photosensitive resin composition. In an implementation, the binder resin in the solid form may be about 0.1 wt % to about 30 wt %, e.g., about 20 wt % to about 30 wt %, based on a total amount of the binder resin solution dissolved in the solvent.

The binder resin may be included in an amount of about 0.1 wt % to about 20 wt %, e.g., about 0.5 wt % to about 15 wt %, or about 1 wt % to about 10 wt %, based on a total solid content of the photosensitive resin composition. Within the above ranges, it is possible to obtain excellent surface smoothness due to excellent developability and improved crosslinking property during manufacture of the color filter.

### (E) Solvent

The solvent may be a material having compatibility with the colorant, the binder resin, the photopolymerizable compound, and the photopolymerization initiator but not reacting therewith.

Examples of the solvent may include alcohols such as methanol, ethanol, and the like; ethers such as dichloroethyl ether, n-butyl ether, diisoamyl ether, methylphenyl ether, tetrahydrofuran, and the like; glycol ethers such as ethylene glycol monomethylether, ethylene glycol monoethylether, and the like; cellosolve acetates such as methyl cellosolve acetate, ethyl cellosolve acetate, diethyl cellosolve acetate, and the like; carbitols such as methylethyl carbitol, diethyl carbitol, diethylene glycol monomethylether, diethylene glycol monoethylether, diethylene glycol dimethylether, diethylene glycol methylethylether, diethylene glycol diethylether, and the like; propylene glycol alkylether acetates such as propylene glycol monomethylether acetate, propylene glycol propylether acetate, and the like; aromatic hydrocarbons such as toluene, xylene and the like; ketones such as methylethylketone, cyclohexanone, 4-hydroxy-4-methyl-2-pentanone, methyl-n-propylketone, methyl-n-butylketone, methyl-n-amylketone, 2-heptanone, and the like; saturated aliphatic monocarboxylic acid alkyl esters such as ethyl acetate, n-butyl acetate, isobutyl acetate, and the like; lactate esters such as methyl lactate, ethyl lactate, and the like; oxy acetic acid alkyl esters such as oxy methyl acetate, oxy ethyl acetate, butyl oxyacetate, and the like; alkoxy acetic acid alkyl esters such as methoxy methyl acetate, methoxy ethyl acetate, methoxy butyl acetate, ethoxy methyl acetate, ethoxy ethyl acetate, and the like; 3-oxy propionic acid alkyl esters such as 3-oxy methyl propionate, 3-oxy ethyl propionate, and the like; 3-alkoxy propionic acid alkyl esters such as 3-methoxy methyl propionate, 3-methoxy ethyl propionate, 3-ethoxy ethyl propionate, 3-ethoxy methyl propionate, and the like; 2-oxy propionic acid alkyl esters such as 2-oxy methyl propionate, 2-oxy ethyl propionate, 2-oxy propyl propionate, and the like; 2-alkoxy propionic acid alkyl esters such as 2-methoxy methyl propionate, 2-methoxy ethyl propionate, 2-ethoxy ethyl propionate, 2-ethoxy methyl propionate, and the like; 2-oxy-2-methyl propionic acid esters such 2-oxy-2-methyl methyl propionate, 2-oxy-2-methyl ethyl propionate, and the like, monooxy monocarboxylic acid alkyl esters of 2-alkoxy-2-methyl alkyl propionates such as 2-methoxy-2-methyl methyl propionate, 2-ethoxy-2-methyl ethyl propionate, and the like; esters such as 2-hydroxy ethyl propionate, 2-hydroxy-2-methyl ethyl propionate, hydroxy ethyl acetate, 2-hydroxy-3-methyl methyl butanoate, and the like; ketonate esters such as ethyl pyruvate, and the like. In an implementation, high boiling point solvent such as N-methylformamide, N,N-dimethylformamide, N-methylformanilide, N-methylacetamide, N,N-dimethylacetamide, N-methylpyrrolidone, dimethylsulfoxide, benzylethylether, dihexylether, acetylacetone, isophorone, caproic acid, caprylic acid, 1-octanol, 1-nonanol, benzylalcohol, benzyl acetate, ethyl benzoate, diethyl oxalate, diethyl maleate, γ-butyrolactone, ethylene carbonate, propylene carbonate, phenyl cellosolve acetate, or the like may be also used.

In an implementation, considering compatibility and reactivity, the solvent may include, e.g., propylene glycol monomethyl ether acetate (PGMEA), n-butyl acetate (n-BA), and ethylene glycol dimethyl ether, or a combination thereof.

The solvent may be included in a balance amount, e.g., about 70 wt % to about 90 wt %, or about 80 wt % to about 90 wt %, based on a total weight of the photosensitive resin composition. Within the above ranges, the photosensitive resin composition has excellent applicability and a coating film with excellent flatness may be obtained.

### (F) Other Additives

In an implementation, the photosensitive resin composition may further include an additive, e.g., malonic acid; 3-amino-1,2-propanediol; a coupling agent containing a vinyl group or a (meth)acryloxy group; or a radical polymerization initiator, in order to help prevent stains or spots during the coating, to help improve leveling performance, and to help prevent the generation of undeveloped residues.

The additives may be adjusted according to desired physical properties.

The coupling agent may be a silane coupling agent, and examples of the silane coupling agent may include trimethoxysilyl benzoic acid, γ methacryl oxypropyl trimethoxysilane, vinyl triacetoxysilane, vinyl trimethoxysilane, γ isocyanate propyl triethoxysilane, γ glycidoxy propyl trimethoxysilane, and β epoxycyclohexyl) ethyltrimethoxysilane, which may be used alone or in mixture of 2 or more types.

The silane coupling agent may be included, e.g., in an amount of 0.01 part by weight to 1 part by weight based on 100 parts by weight of the photosensitive resin composition.

In an implementation, the photosensitive resin composition for a color filter may further include a surfactant, e.g., a fluorine surfactant.

Examples of the fluorine surfactant may include F-482, F-484, and F-478 of DIC Co., Ltd.

The surfactant may be included in an amount of 0.01 wt % to 5 wt %, e.g., 0.01 wt % to 2 wt %, based on a total weight of the photosensitive resin composition. Within the above ranges, a problem in that foreign substances are generated after development may be avoided.

In an implementation, other additives, e.g., an antioxidant, a stabilizer, or the like may be added to the photosensitive resin composition within a range that does not impair physical properties.

### (Photosensitive Resin Layer, Color Filter, and Display Device)

Some example embodiments provide a photosensitive resin layer manufactured using the photosensitive resin composition according to some example embodiments.

The photosensitive resin layer of some example embodiments may be divided into a color positive photoresist composition and a color negative photoresist composition.

The photosensitive resin layer of some example embodiments may be a color negative photoresist. This may have the advantage that coloring caused by the photoresist may not occur and that light sensitivity may be relatively higher than that of positive photoresist.

Some embodiments may provide a color filter manufactured using the aforementioned photosensitive resin composition.

A method of manufacturing the color filter may be as follows.

The aforementioned photosensitive resin composition may be coated to form a photosensitive resin composition layer with a thickness of 0.5 μm to 10 μm on a glass substrate using a suitable method such as spin coating, roller coating, spray coating, or the like.

Subsequently, the substrate having the photosensitive resin composition layer may be irradiated by light (or other radiation or energy) to form a pattern required for a color filter. The irradiation may be performed by using UV, an electron beam, or an X-ray as a radiation source, and the UV may be irradiated, e.g., in a region of 190 nm to 450 nm or 200 nm to 400 nm. The irradiation may be performed by further using a photoresist mask. After performing the irradiation process in this way, the photosensitive resin composition layer exposed to the light source may be treated with a developer. In an implementation, a non-exposed region in the photosensitive resin composition layer may be dissolved and may form the pattern for a color filter. This process may be repeated as many times as the number of desired colors, obtaining a color filter having a desired pattern. In an implementation, the image pattern obtained through development in the above process may be cured by reheating or irradiating an actinic ray thereinto, and crack resistance, solvent resistance, and the like may be improved.

According to some example embodiments, a display device including the aforementioned color filter is provided.

The display device may be a liquid crystal display device, a CMOS image sensor, or the like.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Synthesis Example 1: Compound Represented by Chemical Formula 3-1-1A

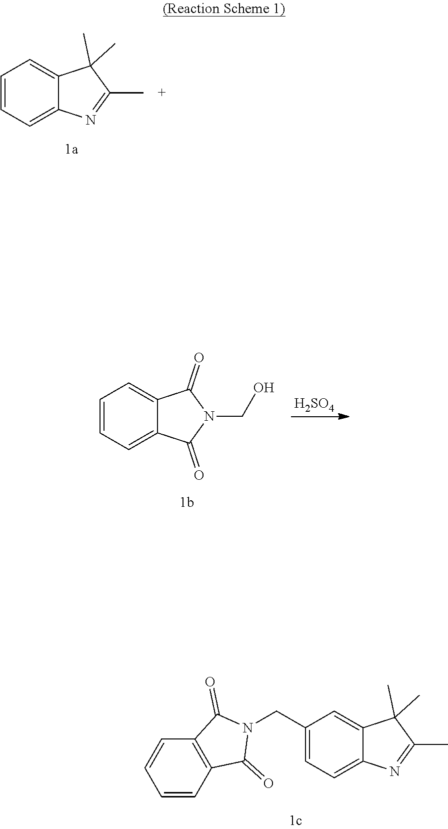

110 mL of sulfuric acid was added to a mixture of 20.0 g of the compound 1a and 20.0 g of the compound 1b and then, stirred at ambient temperature for 72 hours. After cooling in an ice bath, a 10% NaOH (aq) solution was slowly added thereto to produce precipitates. The solids were vacuum-filtered and washed with distilled water. The corresponding solids were recrystallized with n-hexane and dried overnight in a 40° C. vacuum oven to obtain 34 g of the compound 1c.

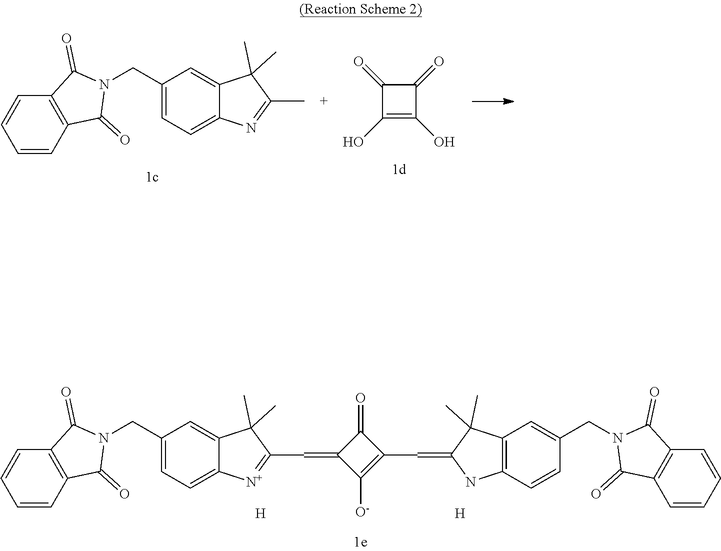

5.00 g of the compound 1c and 895 mg of the compound 1d were dissolved in a mixed solution of n-butanol/toluene (1/1) at ambient temperature and then, heated up to 120° C. and reacted overnight. After removing the solvents under a reduced pressure, n-hexane was added to the remaining reaction mixture and then, stirred at 0° C. for 15 minutes. Subsequently, solids were obtained by filtration under a reduced pressure and then, through column chromatography (eluent: DCM-EtOAc) to obtain 2.24 g of the compound 1e.

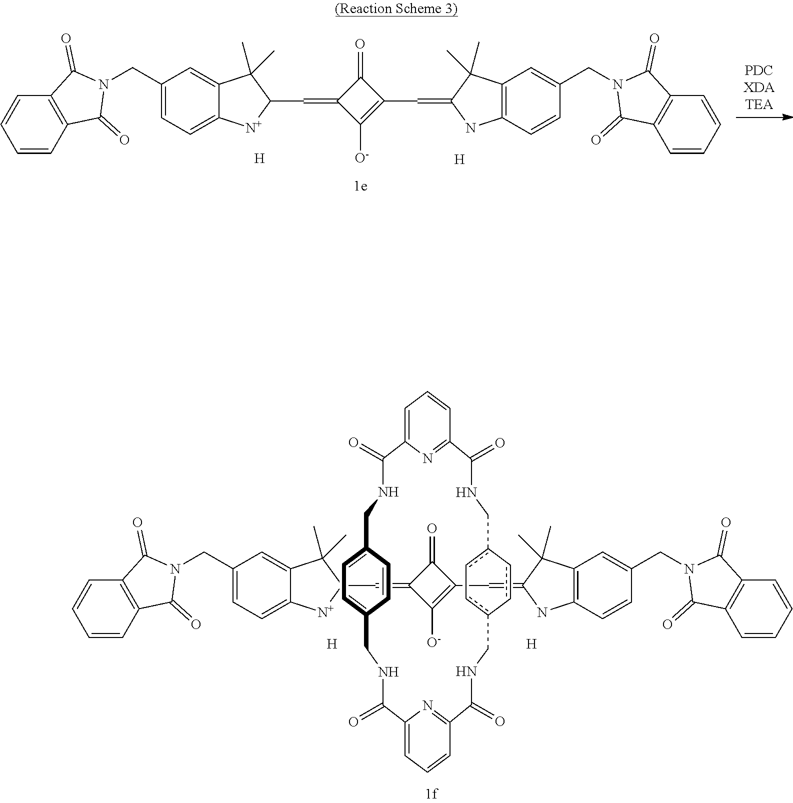

After preparing a solution by dissolving 1.1 g of the compound 1e in chloroform and cooling the solution to 0° C., the following two types of solutions were prepared: (i) one solution prepared by dissolving 0.80 g p-xylxylenediamine (XDA) and 1.2 g of triethylamine in chloroform and (ii) the other solution prepared by dissolving 1.2 g of pyridine-2,6-dicarbonyl dichloride (PDC) in chloroform. These two solutions in the order of (i) and (ii) were slowly added to the solution of the compound 1e and then, stirred for 2 hours at ambient temperature. After removing the solvent under reduced pressure, the residue was redissolved in ethyl acetate to precipitate polymer by-products. After removing the polymer by-products through vacuum filtration, the solvent was removed under reduced pressure. After redissolving the obtained product in chloroform, the above step was performed two additional times. 0.17 g of Compound 1f was obtained by purifying through column chromatography (eluent: DCM-EtOAc).

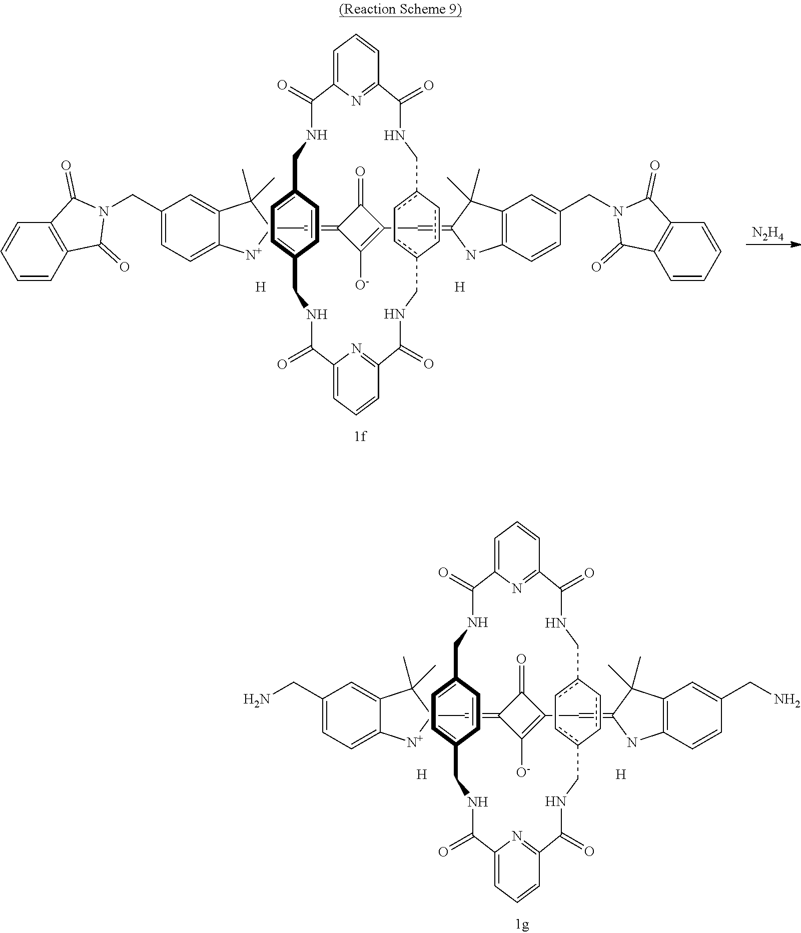

2.00 g of the compound 1f and 801 mg of hydrazine hydrate were added to 10 mL of ethanol and then, stirred at ambient temperature for 1 hour. Subsequently, precipitates produced therein were removed by filtration under a reduced pressure. A filtrate therefrom was concentrated under a reduced pressure to obtain 1.50 g of the compound 1g.

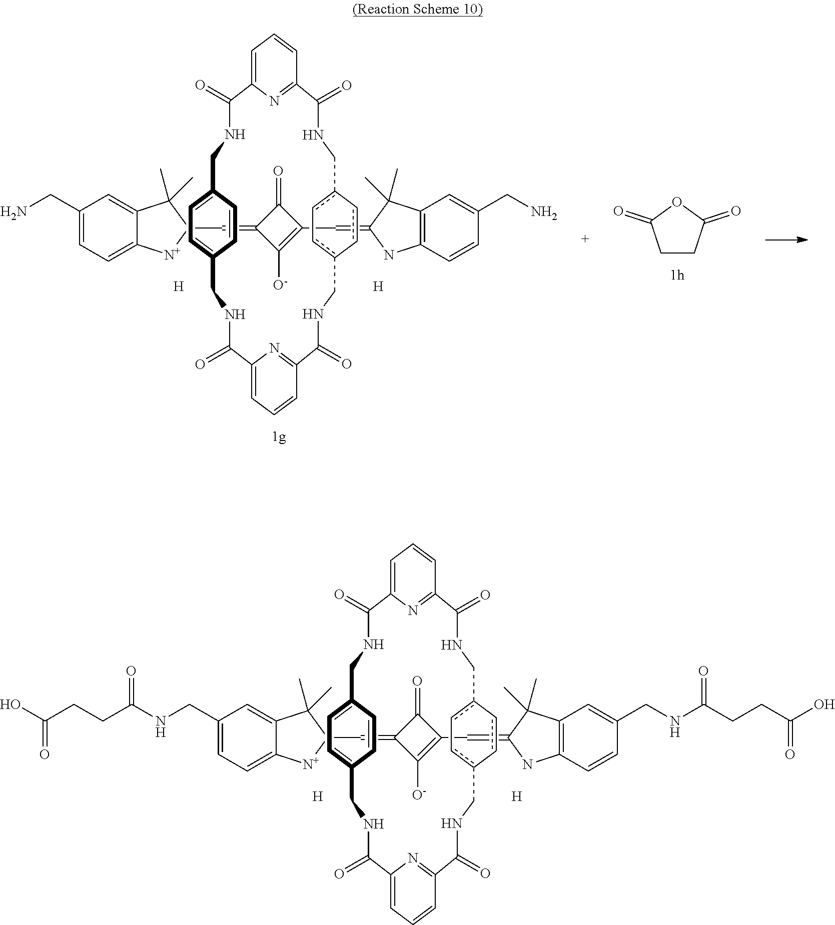

The compound 1g and the compound 1h were added to chloroform and then, stirred at ambient temperature for 1 hour. Subsequently, diethyl ether was added to the reaction mixture to produce solids, and the solids were separated by filtration under a reduced pressure.

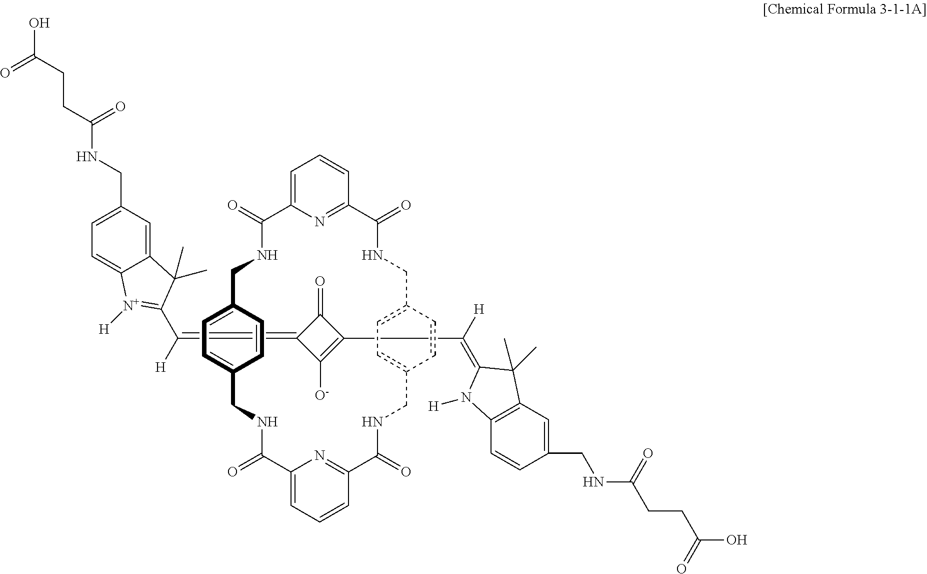

The compound represented by Chemical Formula 3-1-1A had the following MALDI-TOF analysis result.

m/z calcd for C₆₆H₆₅N₁₀O₁₂, 1189.48; found, 1189.48.

### Synthesis Example 2: Compound Represented by Chemical Formula 3-1-1B

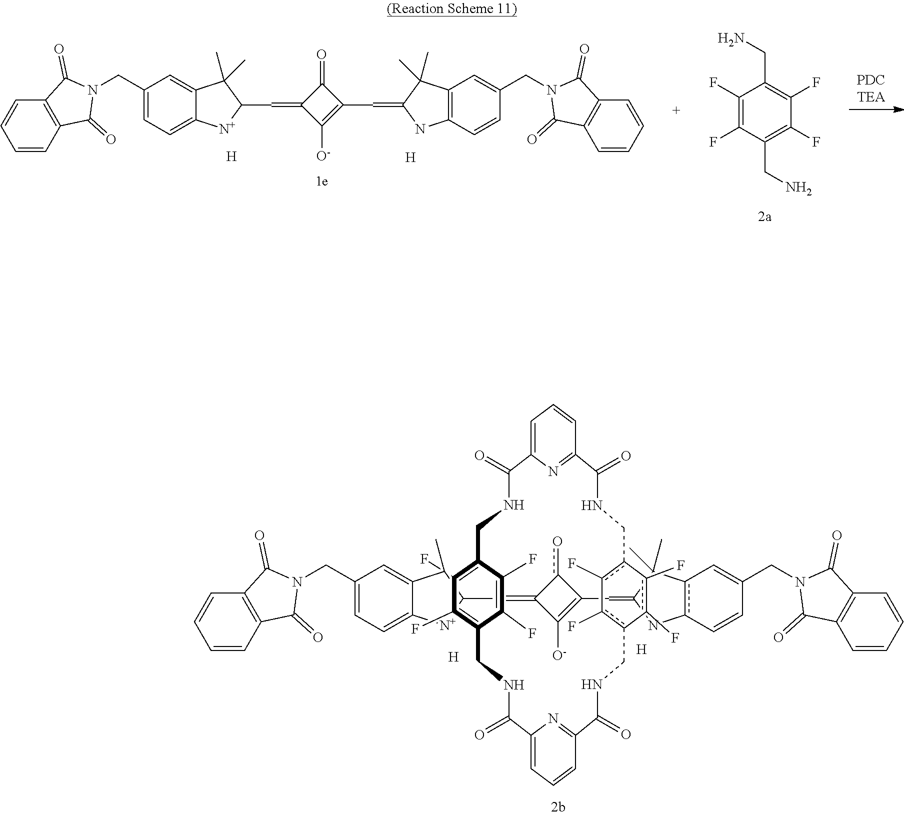

The compound 2b was obtained in the same manner as the synthesis method of the compound 1f except that the compound 2a was used instead of XDA.

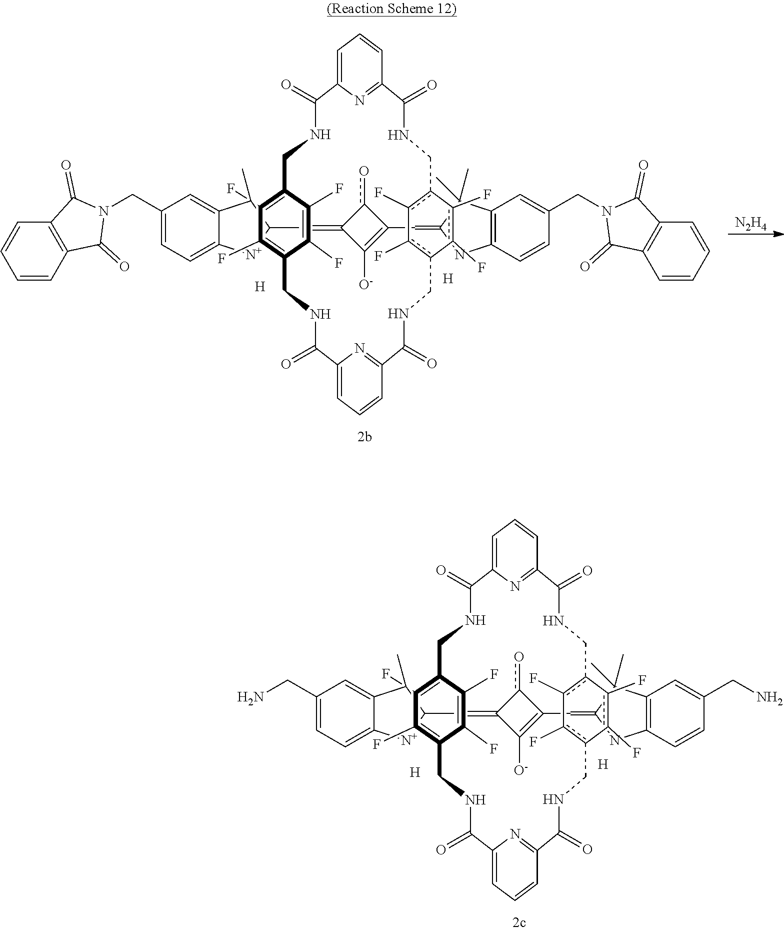

The compound 2c was obtained in the same manner as the synthesis method of the compound 1g.

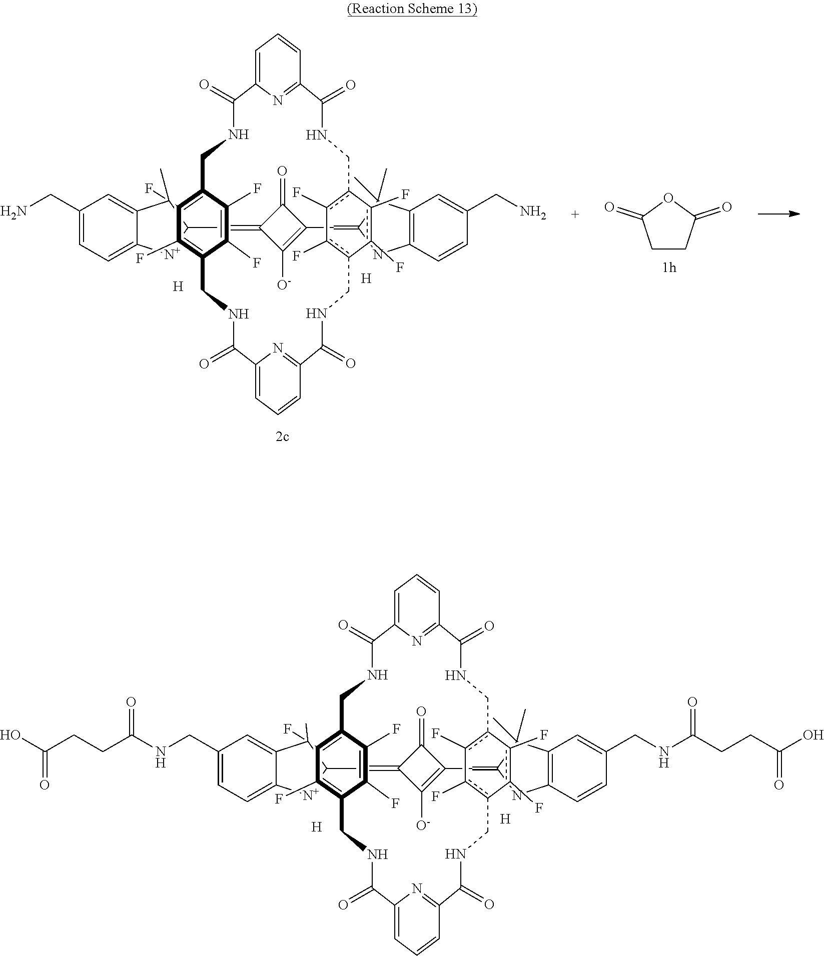

A compound represented by Chemical Formula 3-1-1B was obtained in the same manner as the synthesis method of the compound represented by Chemical Formula 3-1-1A.

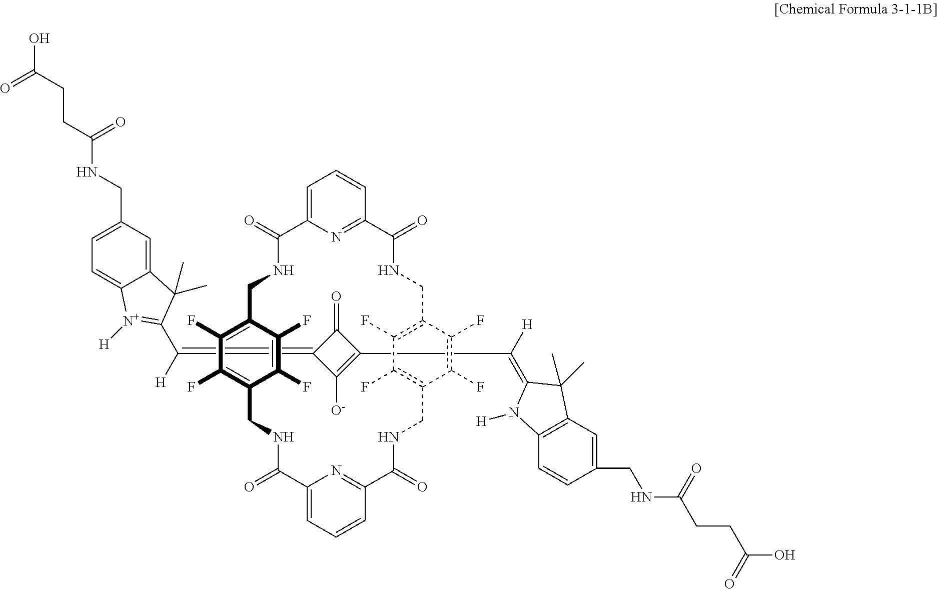

The compound represented by Chemical Formula 3-1-1B had the following

MALDI-TOF analysis result.

m/z calcd for C₆₆H₅₇F₈N₁₀O₁₂, 1333.40; found, 1333.40.

### Synthesis Example 3: Compound Represented by Chemical Formula 3-1-1C

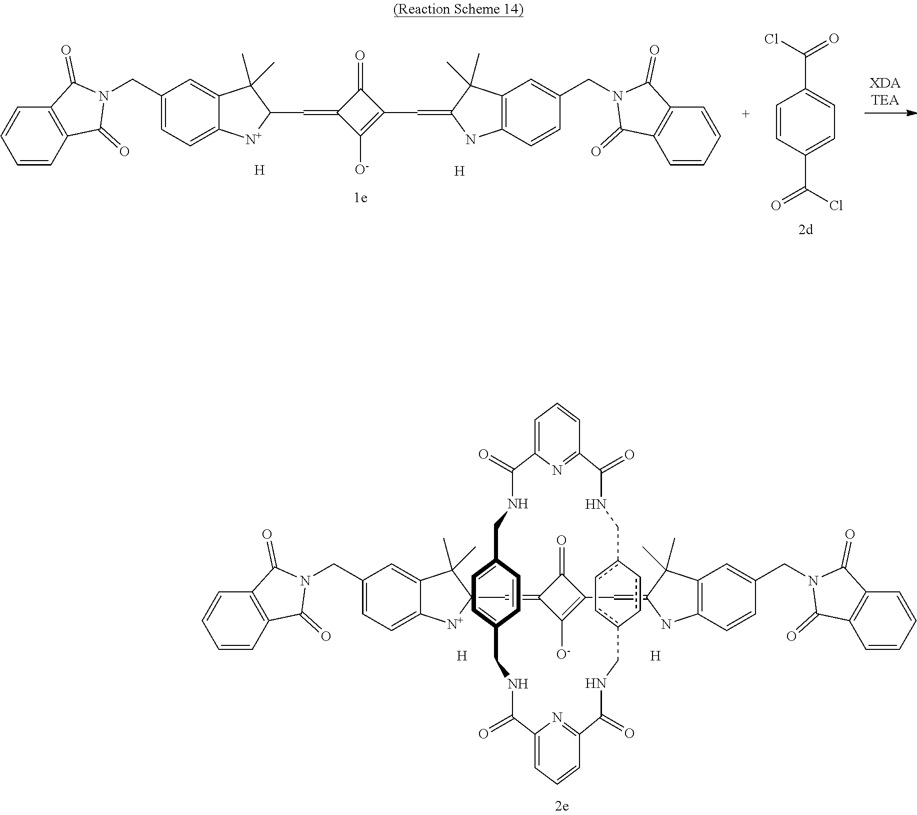

The compound 2e was obtained in the same manner as the synthesis method of the compound 1f except that the compound 2d was used instead of PDC.

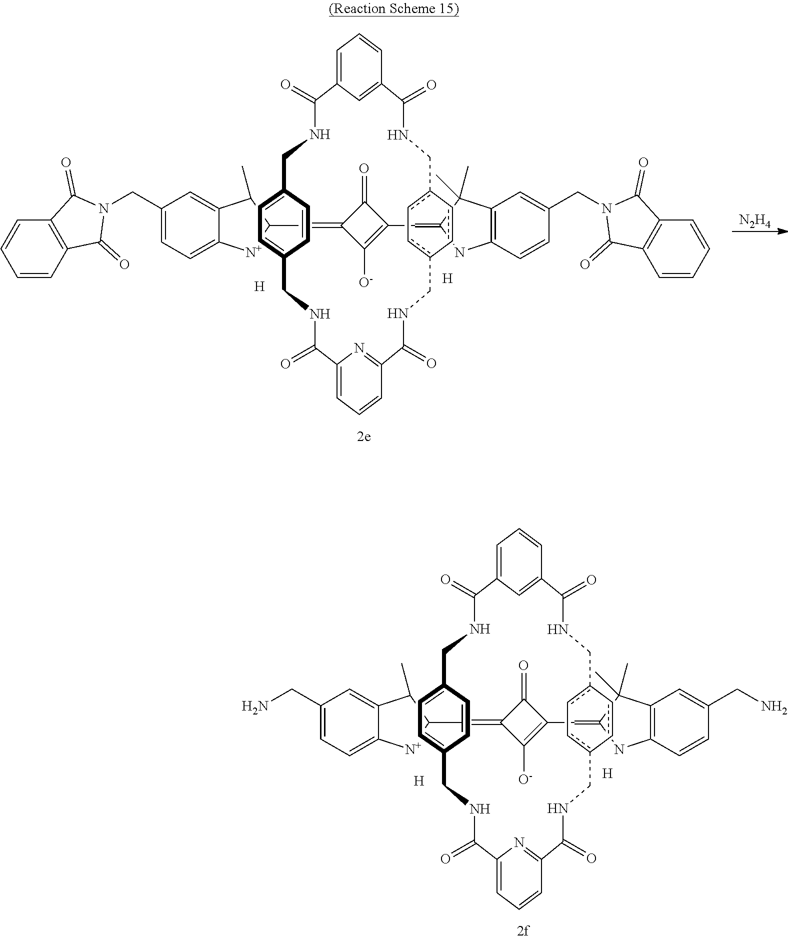

The compound 2f was obtained in a similar manner as the synthesis method of the compound 1g.

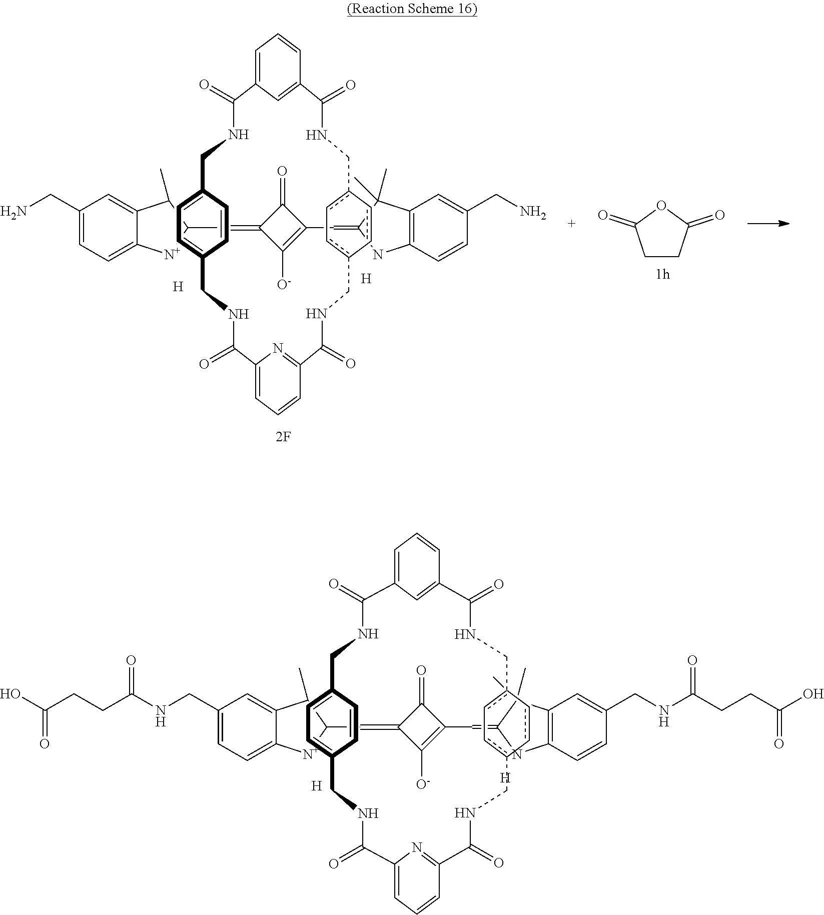

A compound represented by Chemical Formula 3-1-1C was obtained in a similar manner as the synthesis method of the compound represented by Chemical Formula 3-1-1A.

The compound represented by Chemical Formula 3-1-1C had the following MALDI-TOF analysis result.

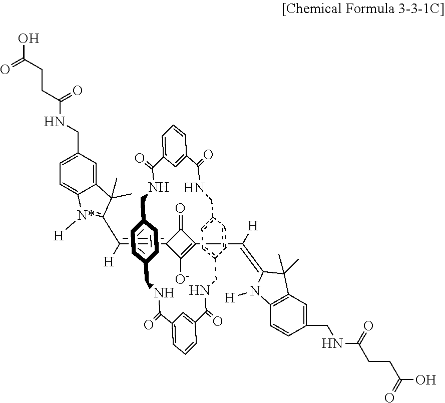

m/z calcd for (C₆₈H₆₇N₈O₁₂), 1187.49; found, 1187.49.

### Synthesis Example 4: Compound Represented by Chemical Formula 3-1-1D

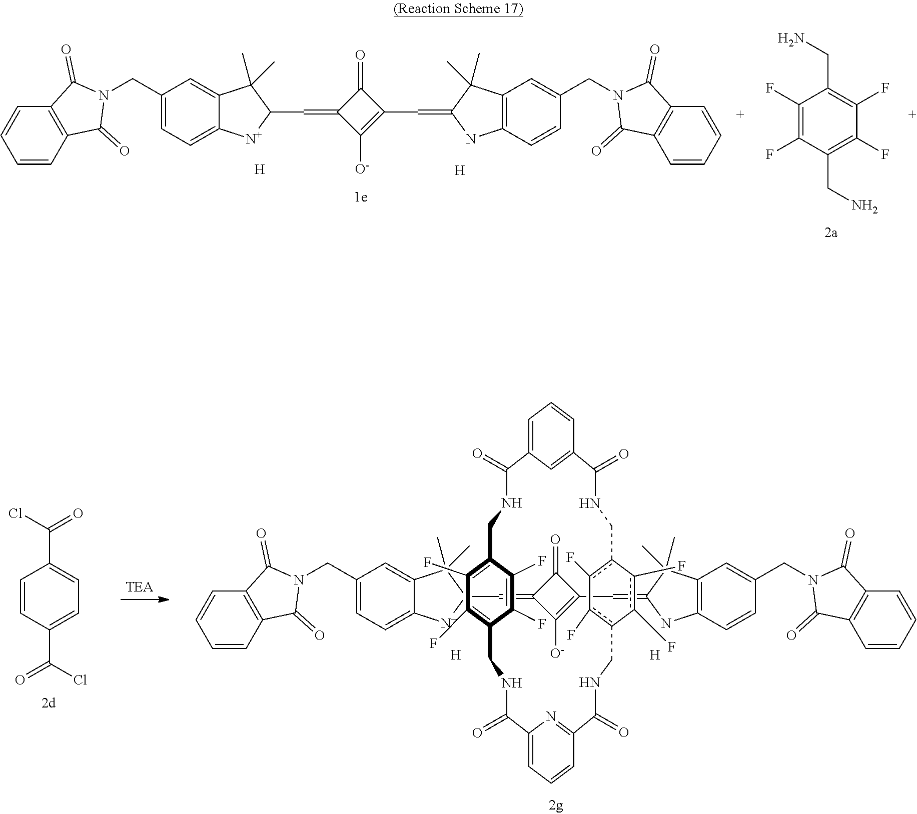

The compound 2g was obtained in the same manner as the synthesis method of the compound 1f except that the compound 2a instead of XDA and the compound 2d instead of PDC were used.

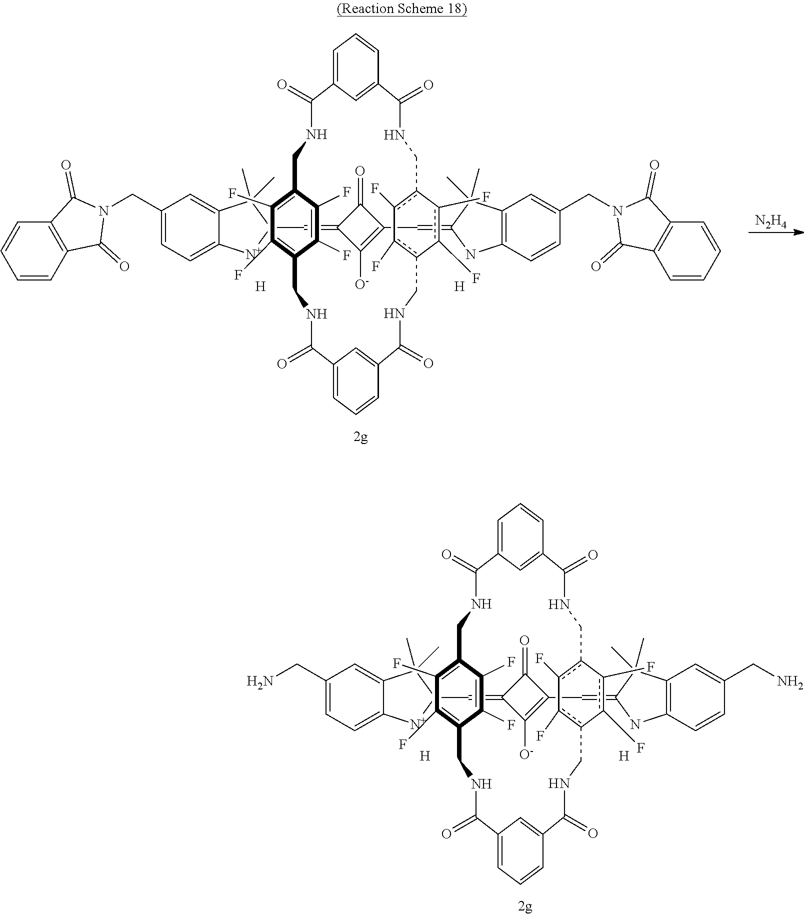

The compound 2h was obtained in a similar manner as the synthesis method of the compound 1g.

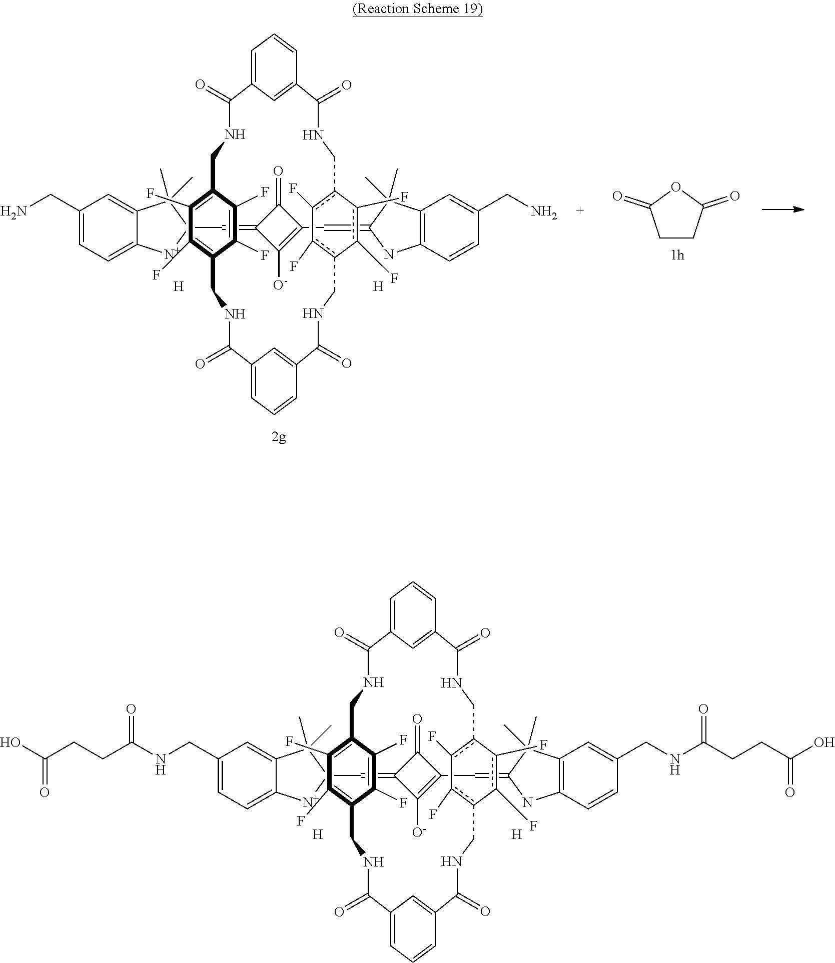

A compound represented by Chemical Formula 3-1-1D was obtained in the same manner as the synthesis method of the compound 1 represented by Chemical Formula 3-1-1A.

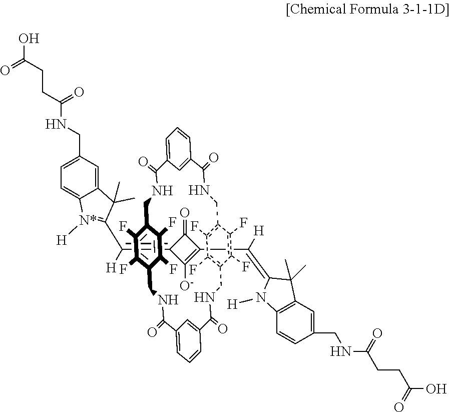

The compound represented by Chemical Formula 3-1-1D had the following MALDI-TOF analysis result.

m/z calcd for C₆₈H₅₉F₈N₈O₁₂, 1331.41; found, 1331.41.

### Synthesis Example 5: Compound Represented by Chemical Formula 3-1-1E

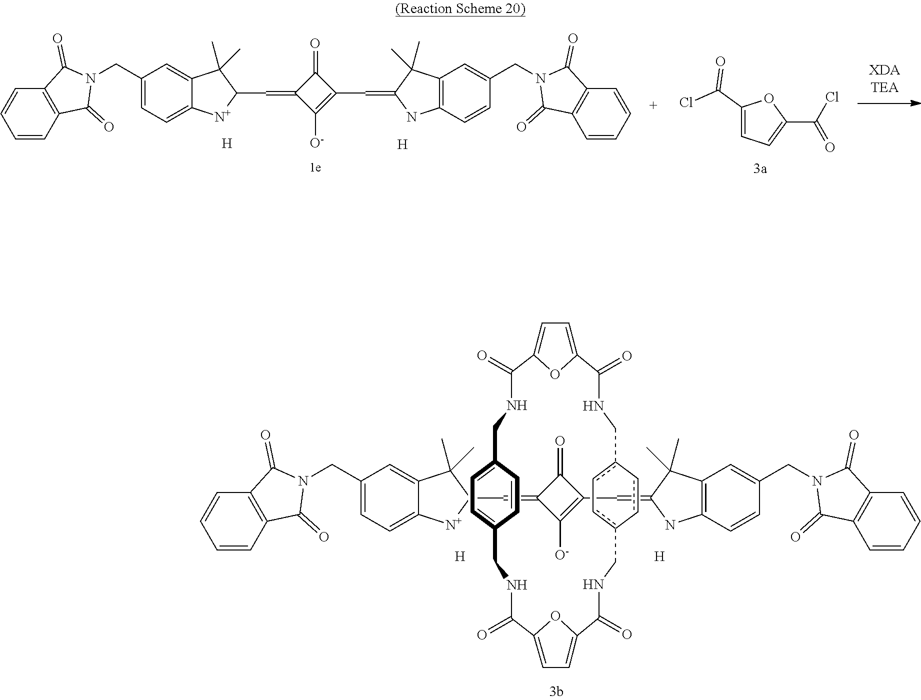

The compound 3b was obtained in the same manner as the synthesis method of the compound 1f except that the compound 3a was used instead of PDC.

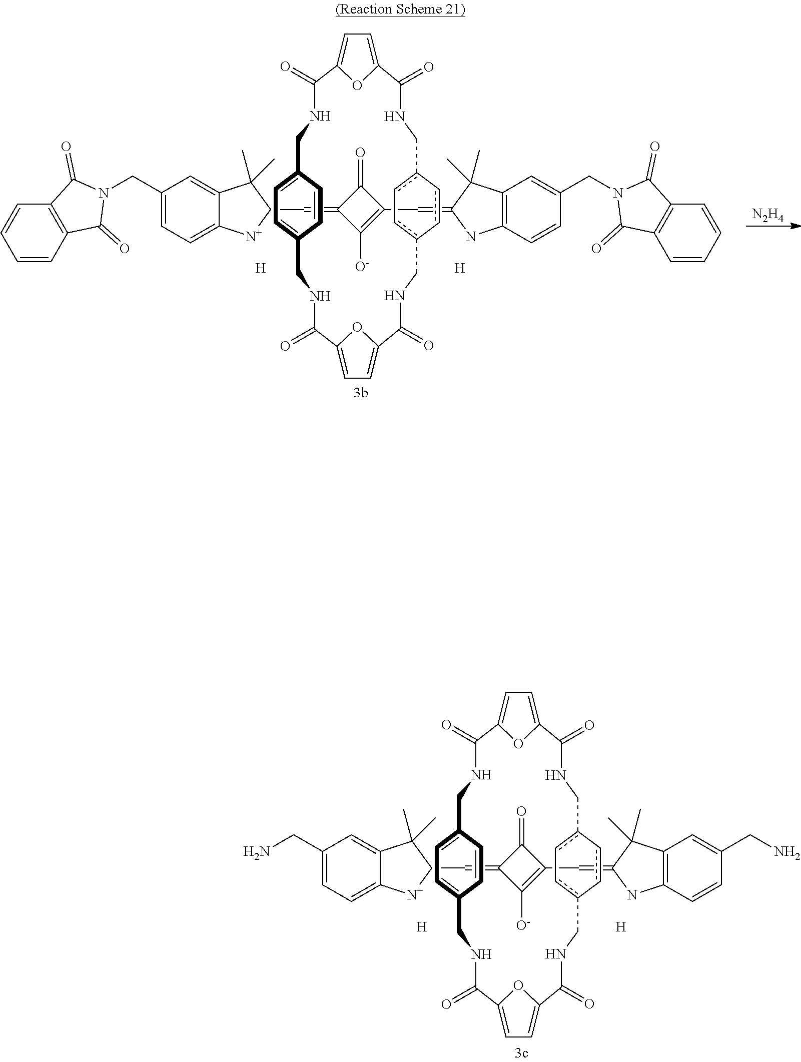

The compound 3c was obtained in a similar manner as the synthesis method of the compound 1g.

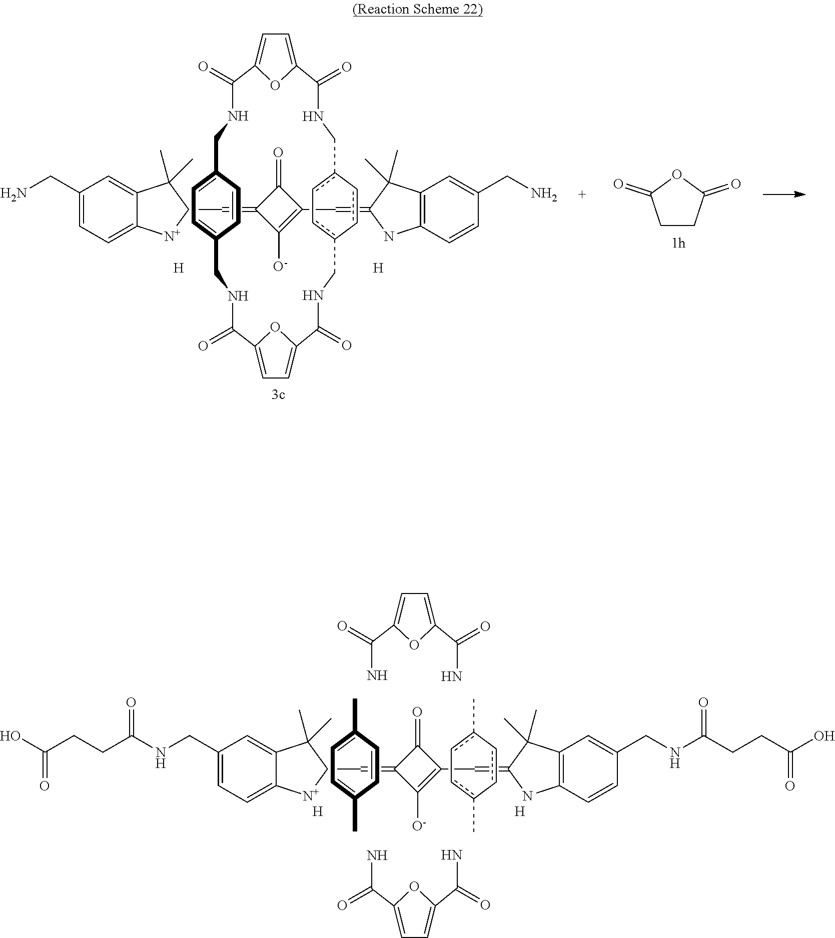

A compound represented by Chemical Formula 3-1-1E was obtained in a similar manner as the synthesis method of the compound 1 represented by Chemical Formula 3-1-1A.

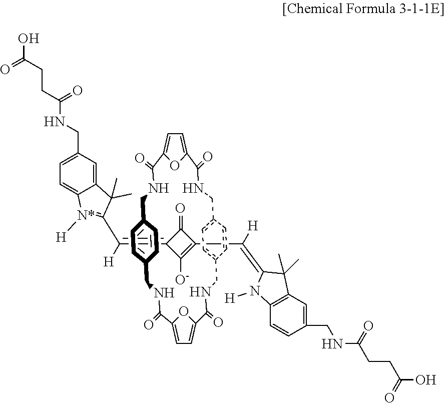

The compound represented by Chemical Formula 3-1-1E had the following MALDI-TOF analysis result.

m/z calcd for C₆₄H₆₃N₈O₁₄, 1167.45; found, 1167.45.

### Synthesis Example 5: Compound Represented by Chemical Formula 3-1-1F

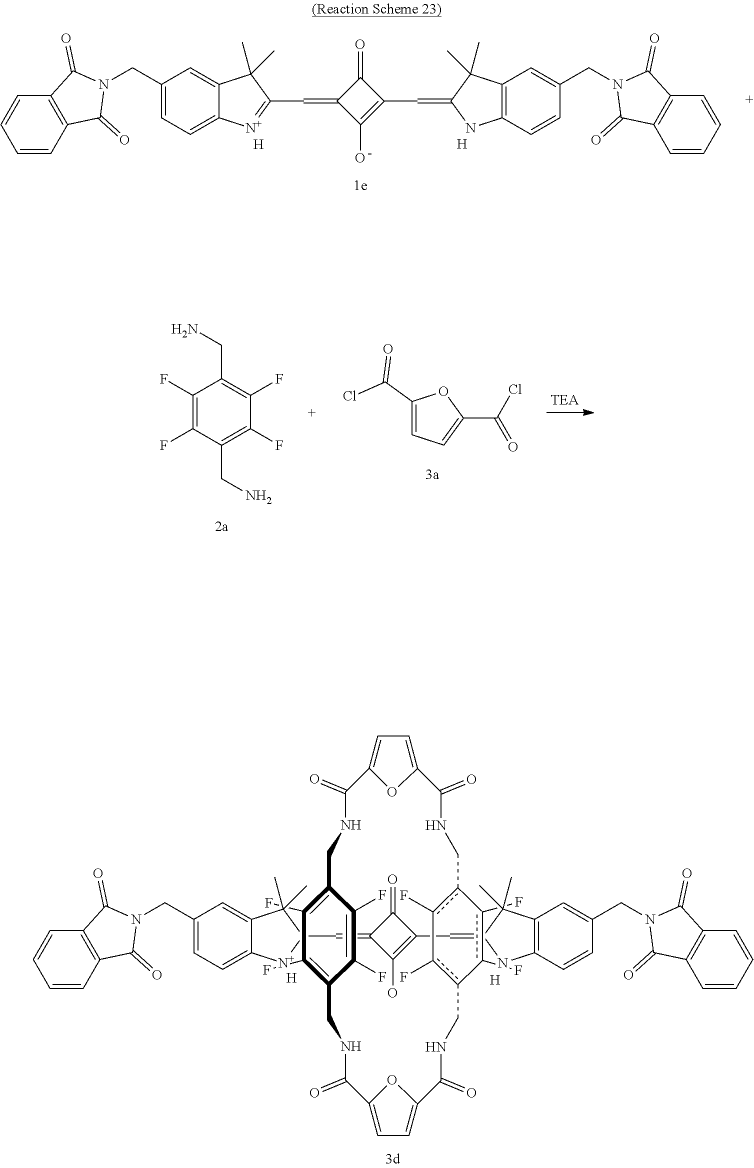

The compound 3d was obtained in the same manner as the synthesis method of the compound 1f except that the compound 2a instead of XDA and the compound 3a instead of PDC were used.

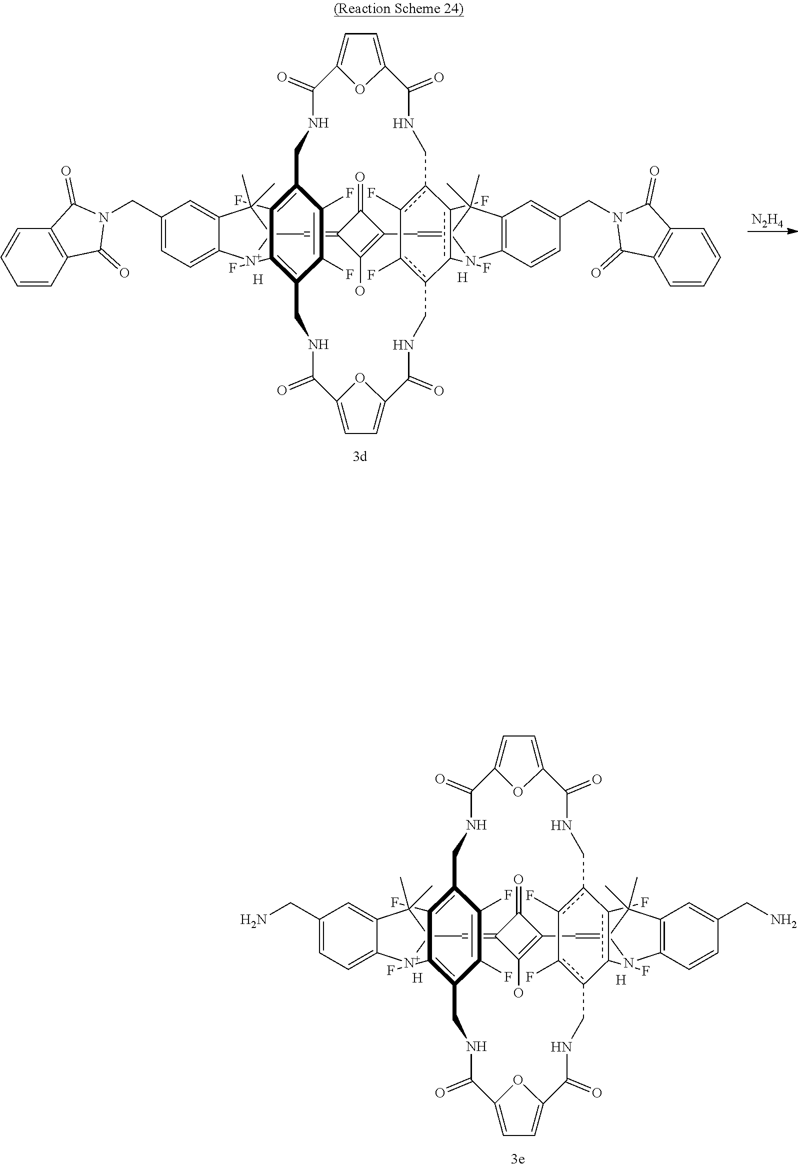

The compound 3e was obtained in a similar manner as the synthesis method of the compound 1g.

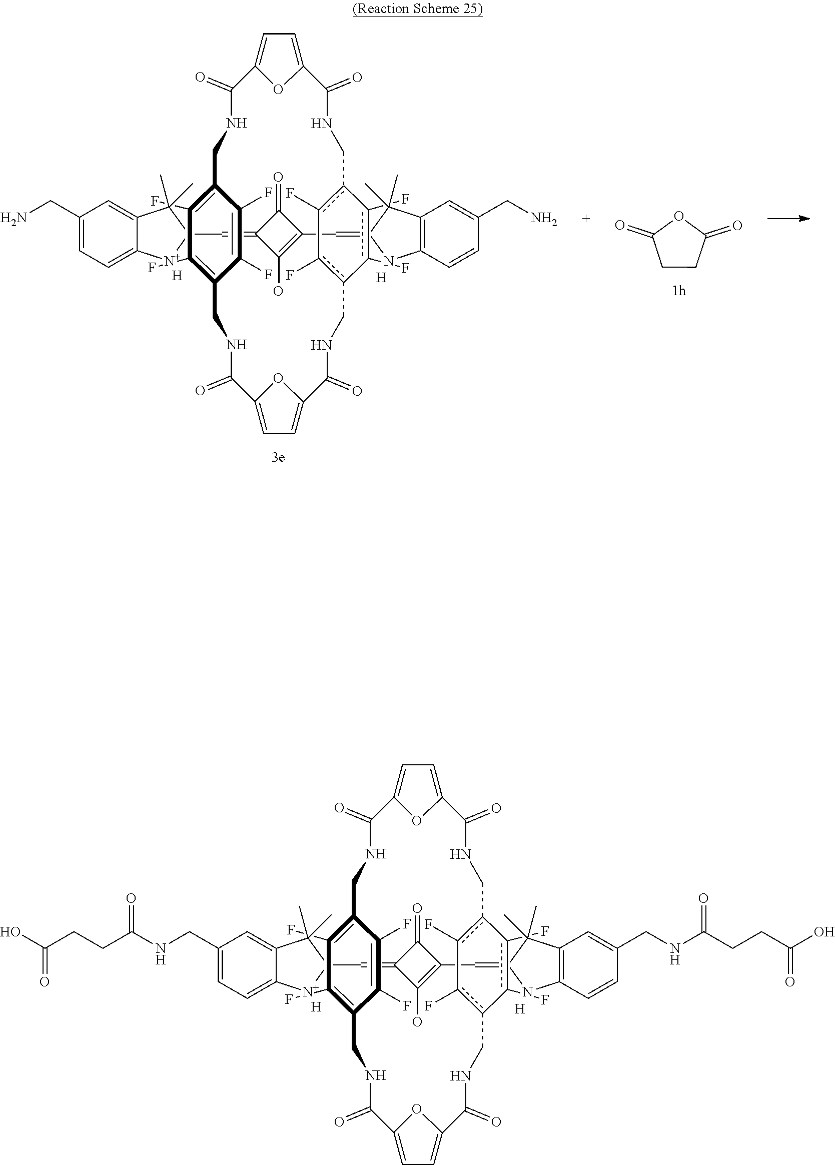

A compound represented by Chemical Formula 3-1-1F was obtained in a similar manner as the synthesis method of the compound represented by Chemical Formula 3-1-1A.

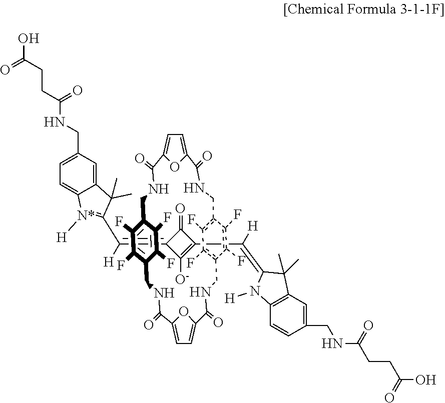

The compound represented by Chemical Formula 3-1-1F had the following MALDI-TOF analysis result.

m/z calcd for C₆₄H₅₅F₈N₈O₁₄, 1311.37; found, 1311.37.

### Synthesis Example 7: Compound Represented by Chemical Formula 3-2-1A

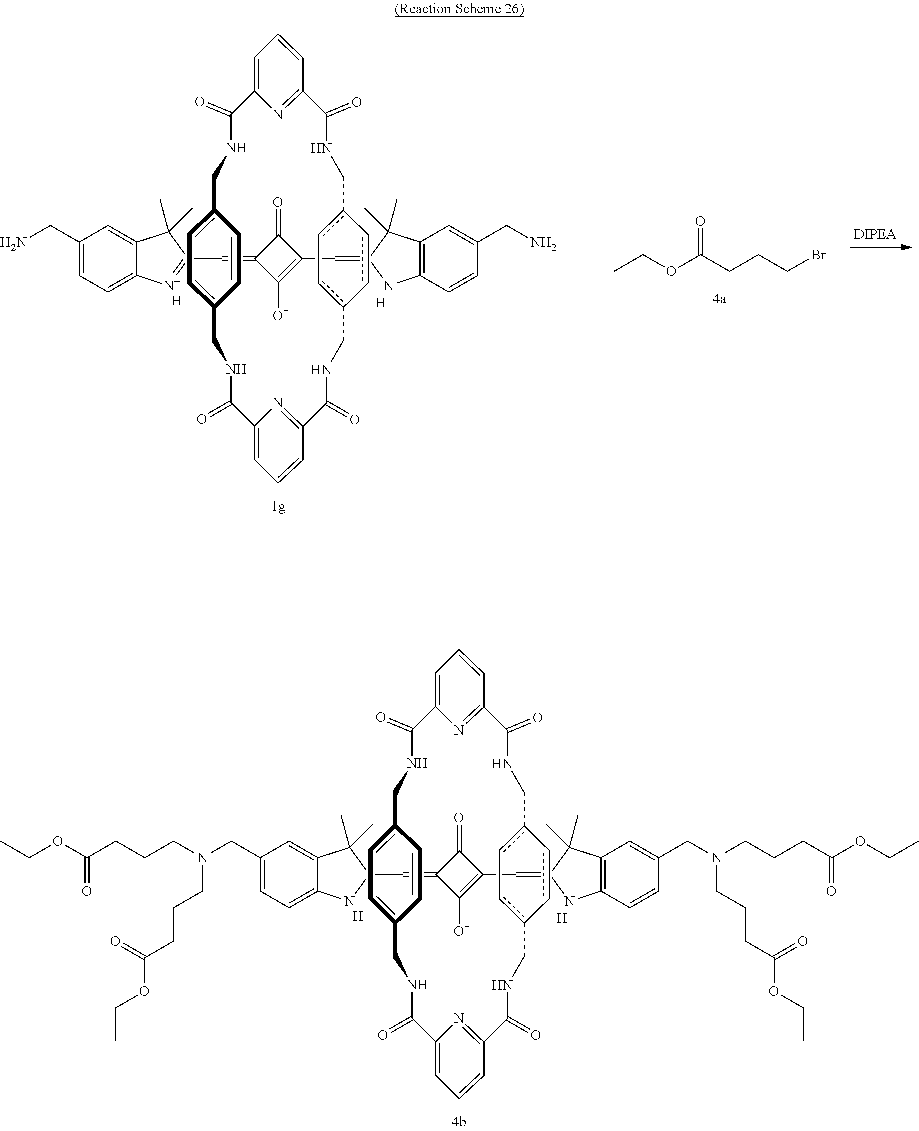

The compound 1g and the compound 4a with DIPEA were dissolved in acetonitrile and then, stirred at 120° C. overnight in a high-pressure tube. After removing the solvent under a reduced pressure, the residue was redissolved in dichloromethane and then, filtered to remove a salt. After removing the solvent again under a reduced pressure, solids were precipitated with diethyl ether and recovered by filtration to obtain the compound 4b.

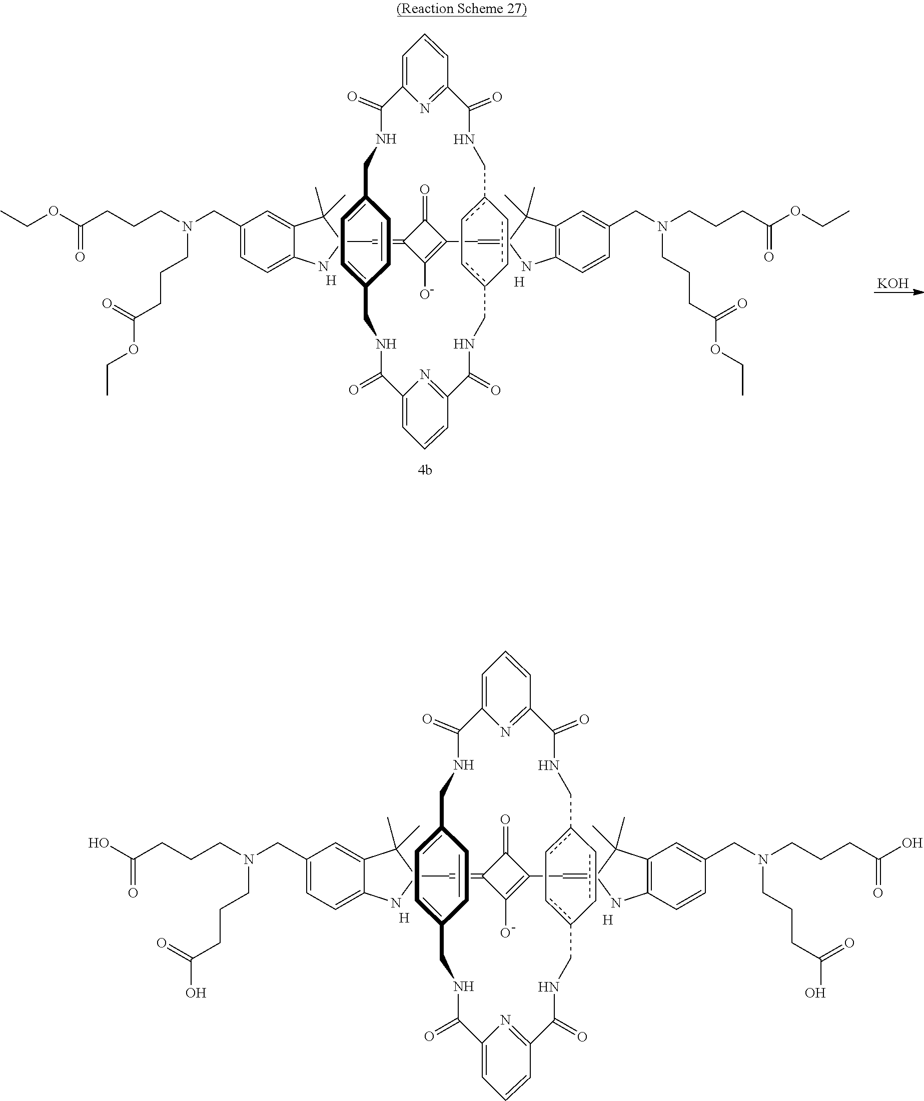

The compound 4b was dissolved in THF, and 0.2 N KOH (aq) was added thereto and then, stirred overnight at ambient temperature. The resultant was acidified (pH 1 to 2) by using 1 N HCl (aq) and then, extracted with DCM. An organic layer therefrom was passed through MgSO₄ and concentrated to obtain a compound represented by Chemical Formula 3-2-1A.

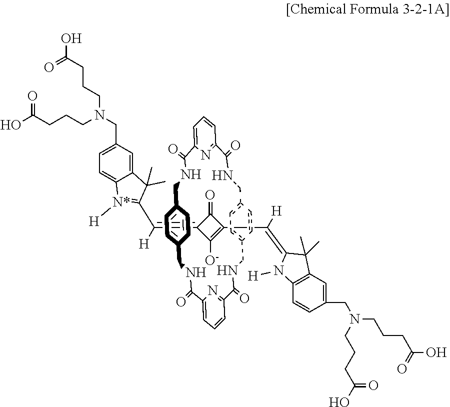

The compound represented by Chemical Formula 3-2-1A had the following MALDI-TOF analysis result.

m/z calcd for C₇H₈₁N₁₀O₁₄, 1333.59; found, 1333.59.

### Synthesis Example 8: Compound Represented by Chemical Formula 3-3-1A

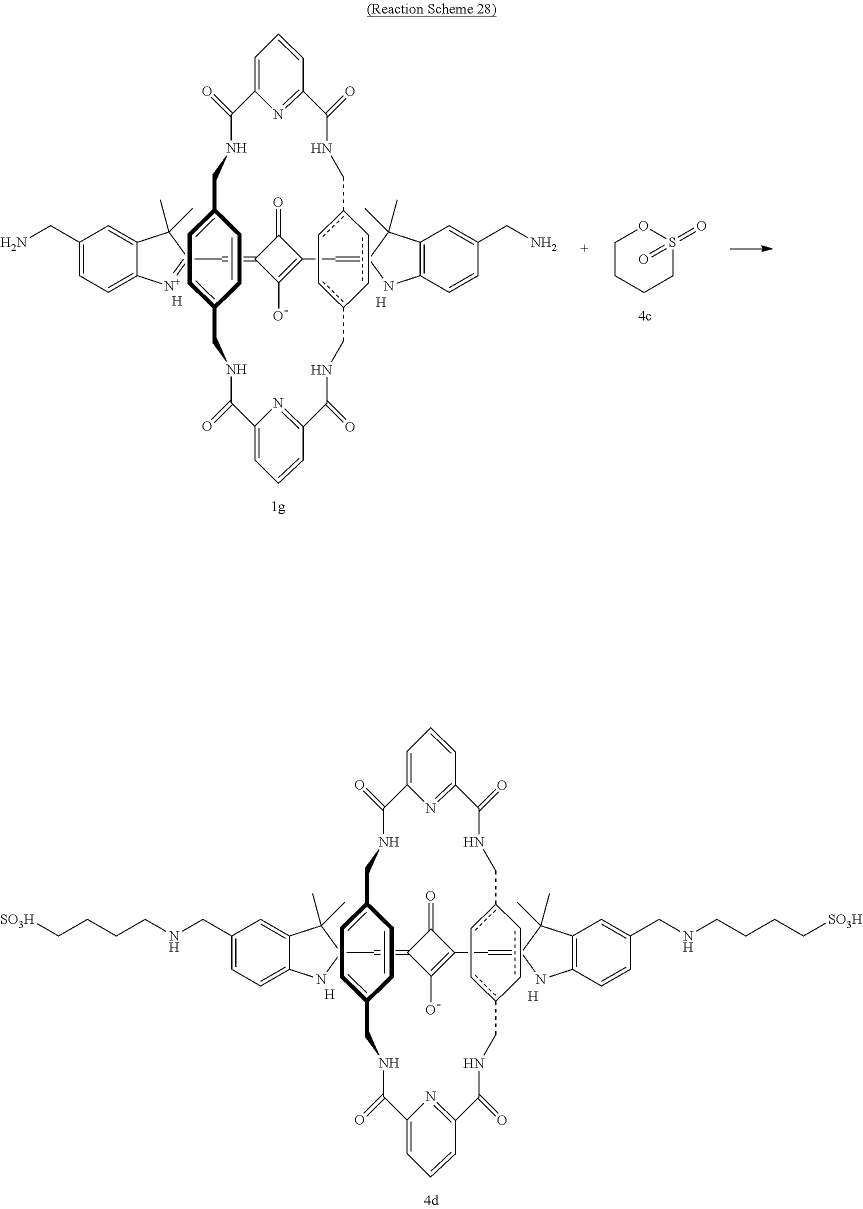

The compound 1g and the compound 4c were dissolved in acetonitrile and then, stirred overnight at 120° C. in a high-pressure tube. After removing the solvent under a reduced pressure, solids were precipitated therefrom with diethyl ether, filtered, and washed with diethyl ether to obtain the compound 4d.

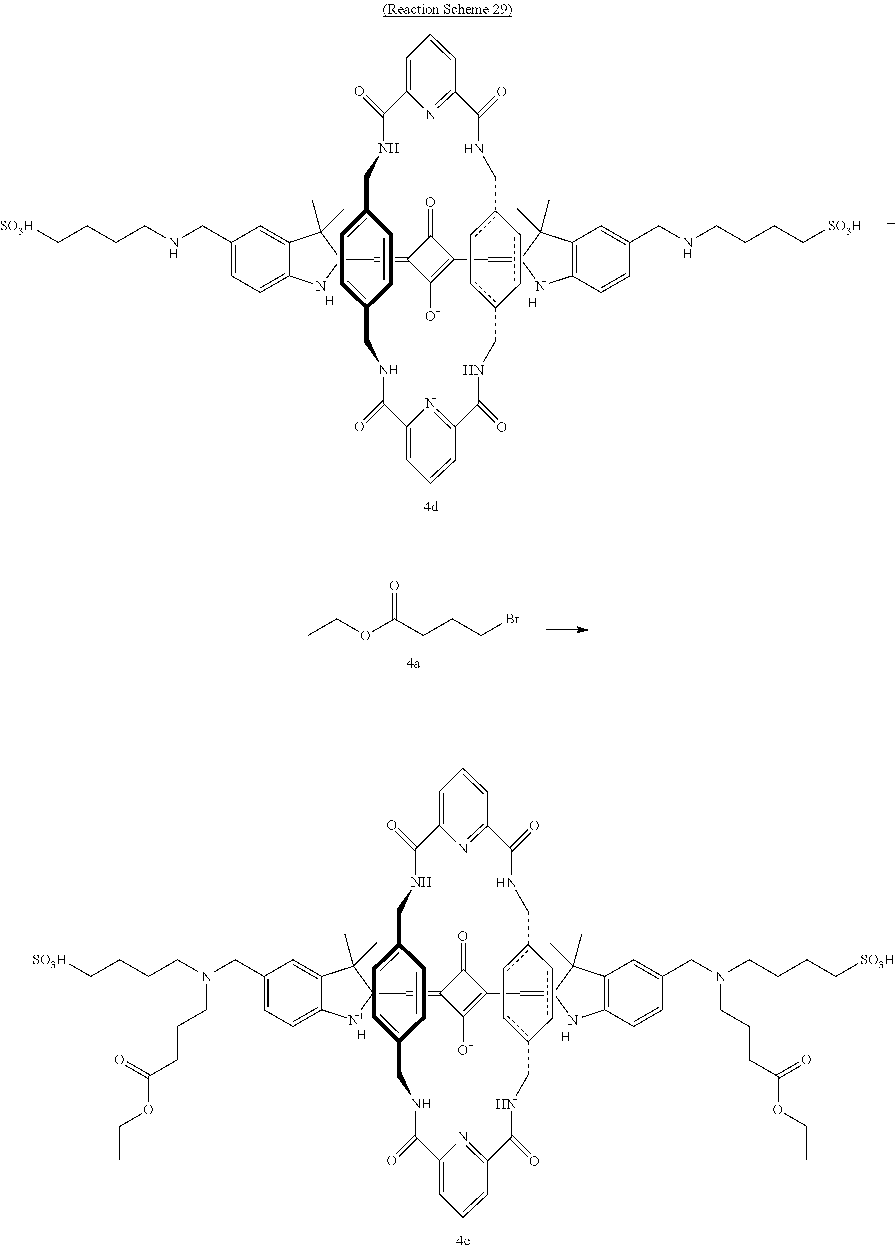

The compound 4e was obtained in a similar manner as the synthesis method of the compound 4b.

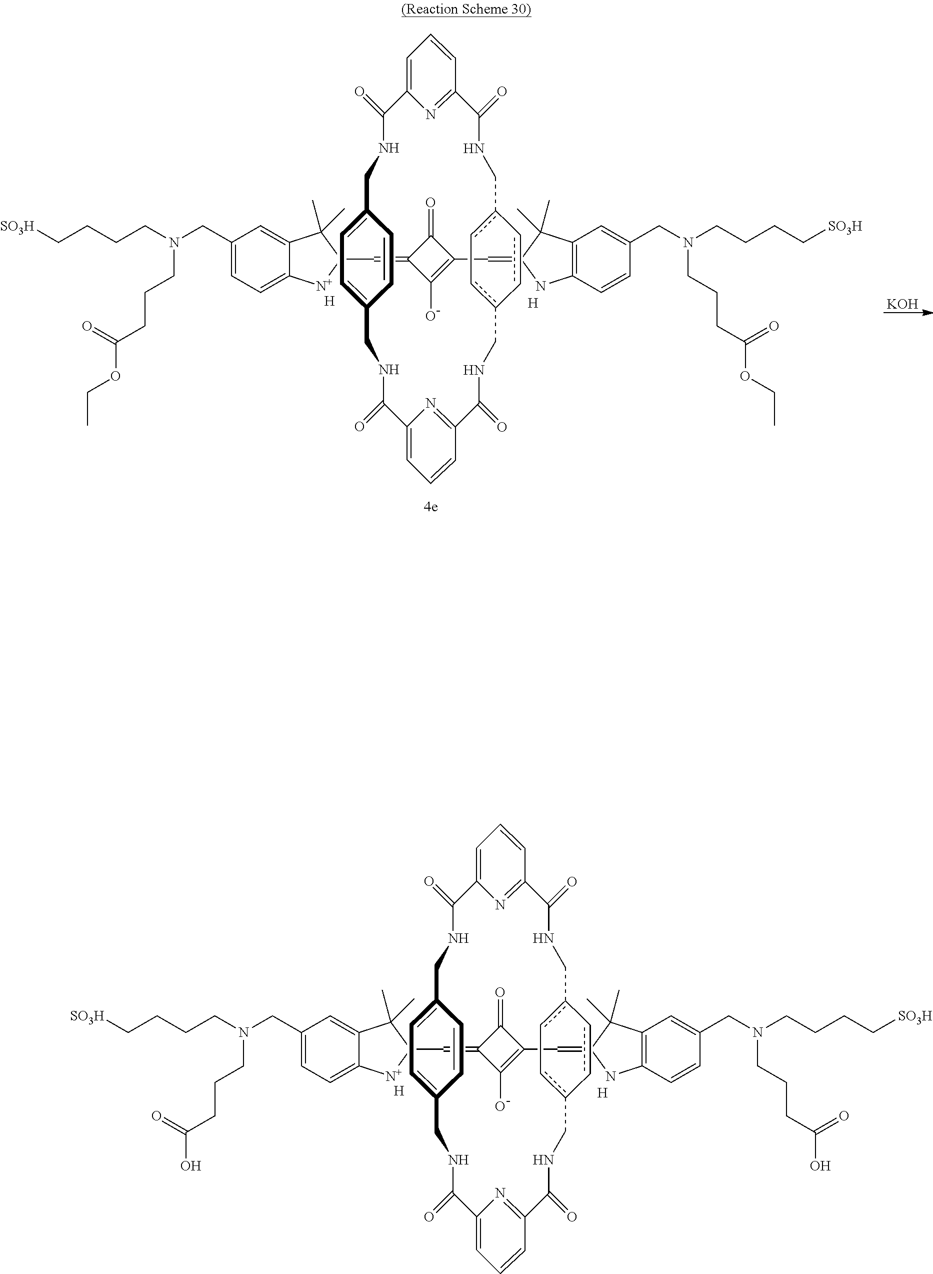

A compound represented by Chemical Formula 3-3-1A was obtained in a similar manner as the synthesis method of the compound represented by Chemical Formula 3-2-1A.

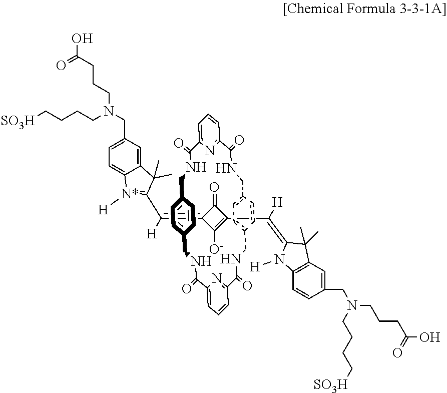

The compound represented by Chemical Formula 3-3-1A had the following MALDI-TOF analysis result.

m/z calcd for C74H85N10016S2, 1433.56; found, 1433.56.

### Synthesis Example 9: Compound Represented by Chemical Formula 3-4-1A

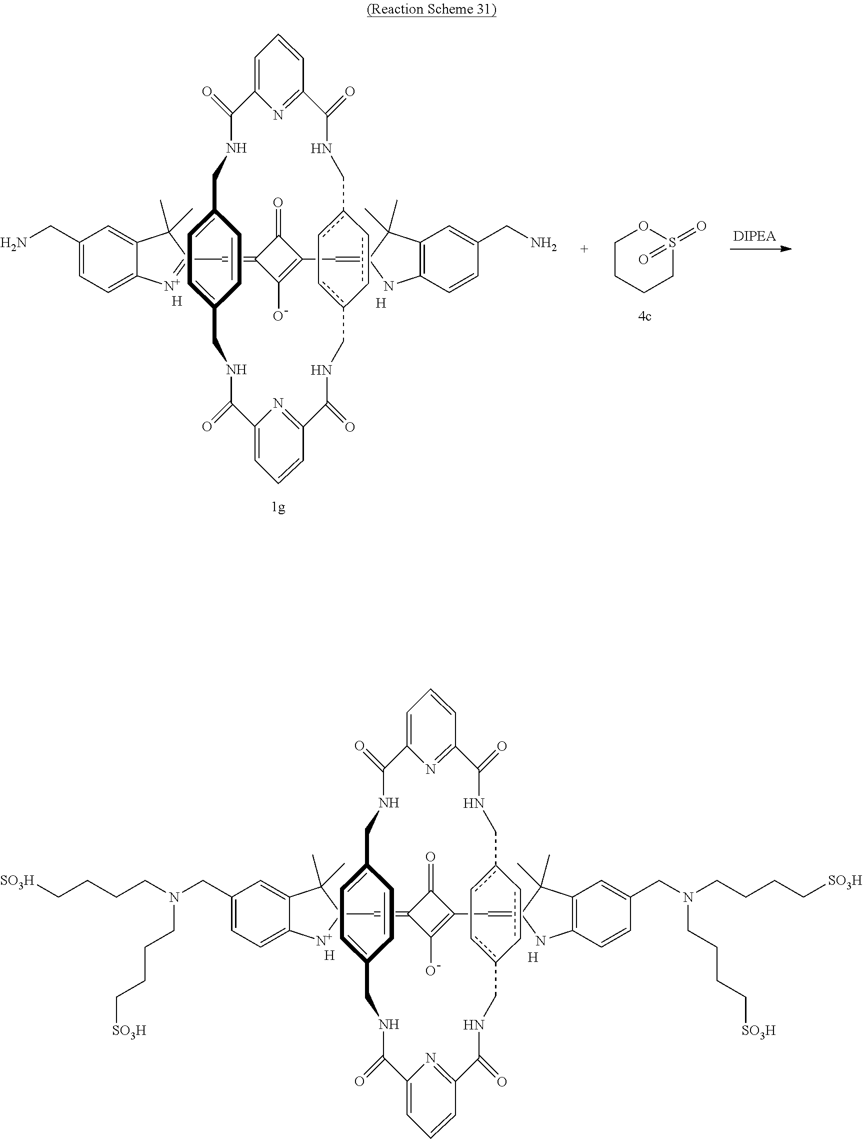

A compound represented by Chemical Formula 3-4-1A was obtained in the same manner as the synthesis method of the compound 4b except that the compound 4c was used instead of the compound 4a.

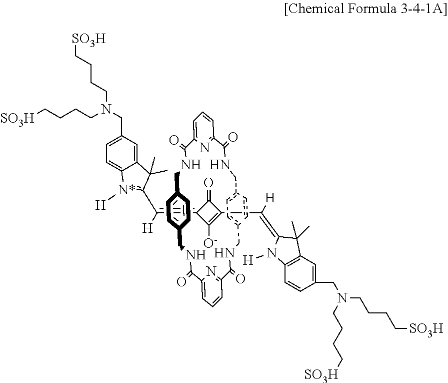

The compound represented by Chemical Formula 3-4-1A had the following MALDI-TOF analysis result.

m/z calcd for C74H39N10018S4, 1533.52; found, 1533.52.

### Comparative Synthesis Example 1: Compound Represented by Chemical Formula X

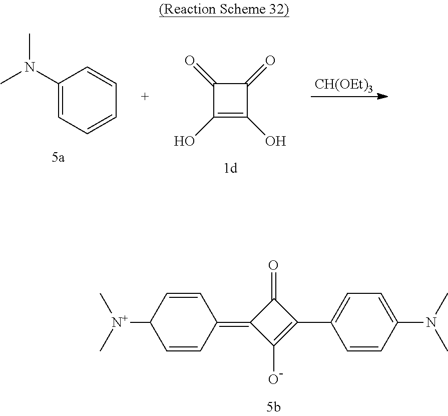

2.93 g of the compound 5a and 12.1 mL of triethyl orthoformate were dissolved in n-amyl alcohol, and 1.93 g of the compound 1d was added thereto at ambient temperature. The corresponding solution was stirred at 90° C. for 4 hours, and a reaction progress thereof was checked through TLC. After completely removing the solvent under a reduced pressure, the residue was dispersed in n-hexane. The reaction mixture was cooled to 0° C. for 15 minutes to precipitate solids. The precipitates were separated from the mixture through vacuum filtration and washed by using n-hexane. 5.64 g of Compound 5b was used without additional purification in the following reaction step.

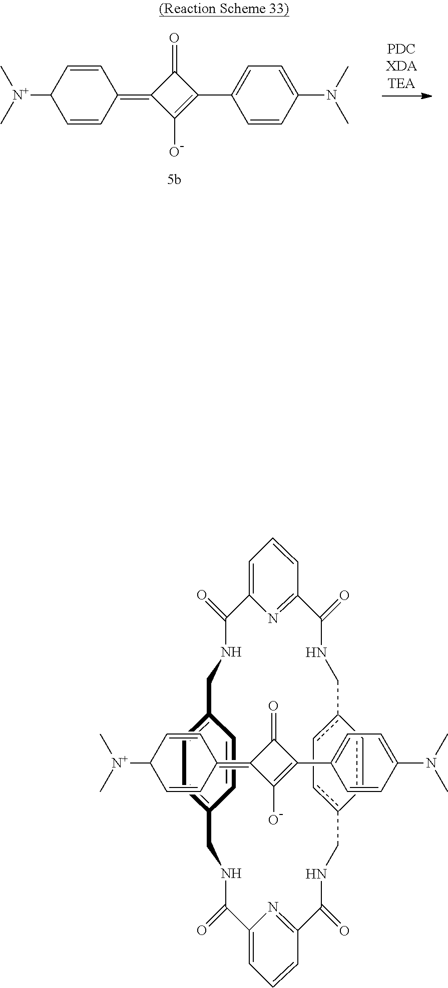

A compound represented by Chemical Formula X was obtained in a similar manner as the synthesis method of the compound represented by Chemical Formula 3-1-1A.

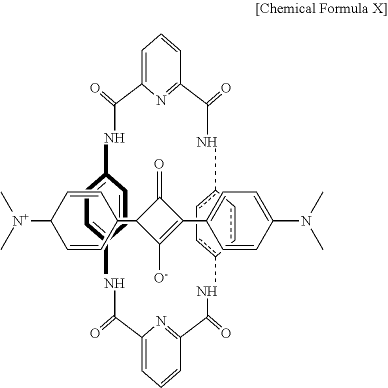

The compound represented by Chemical Formula X had the following MALDI-TOF analysis result.

m/z calcd for C₅₀H₄₇N₈O₆, 855.36; found, 855.36.

### Comparative Synthesis Example 2: Compound Represented by Chemical Formula Y

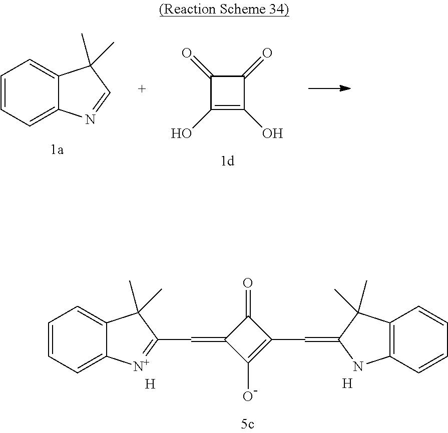

The compound 2f was obtained in a similar manner as the synthesis method of the compound 1e.

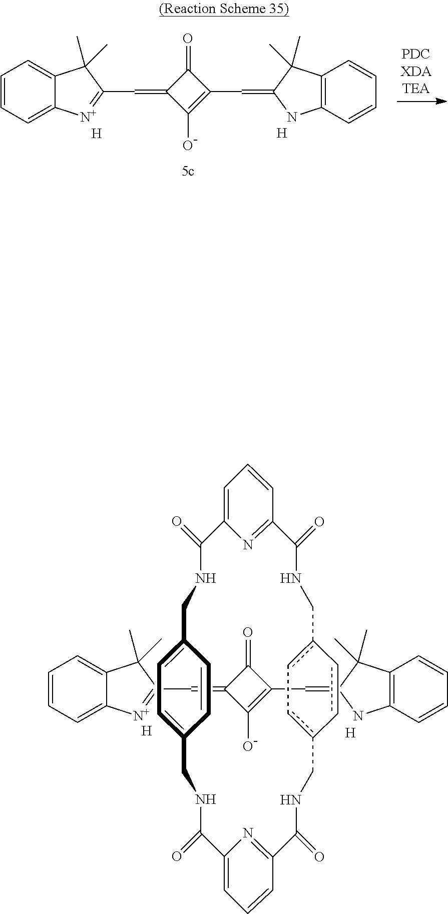

A compound represented by Chemical Formula Y was obtained in a similar manner as the synthesis method of the compound represented by Chemical Formula 3-1-1A.

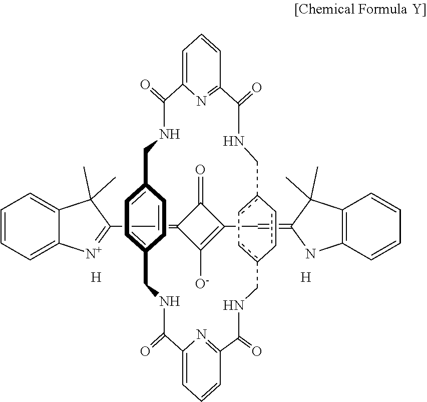

The compound represented by Chemical Formula Y had the following MALDI-TOF analysis result.

m/z calcd for C₅₆H₅₁N₈O₆, 931.39; found, 931.39.

### Comparative Synthesis Example 3: Compound Represented by Chemical Formula Z

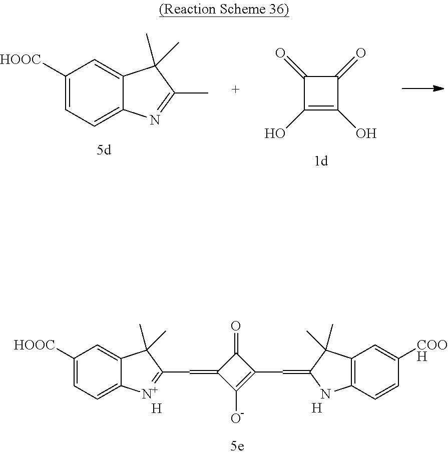

The compound 5e was obtained in the same manner as the synthesis method of the compound 1e.

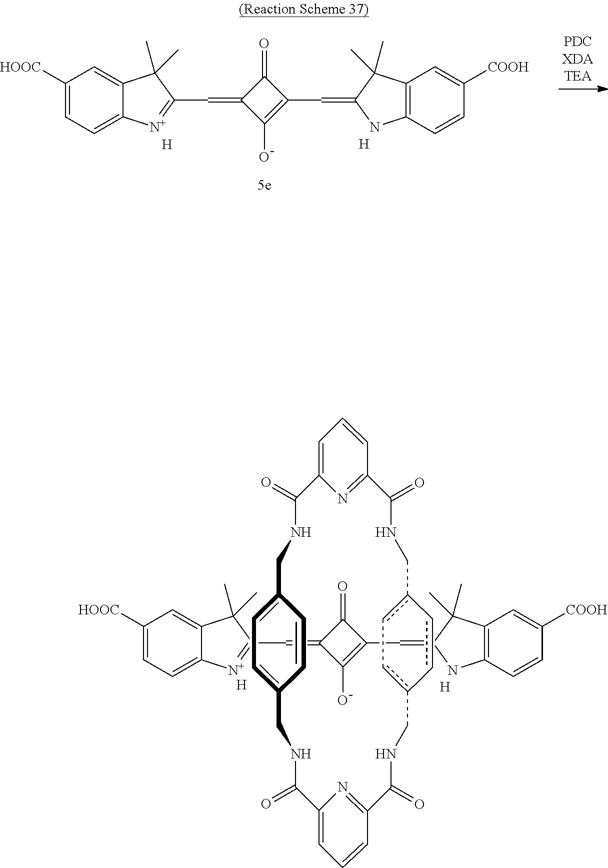

A compound represented by Chemical Formula Z was obtained in a similar manner as the synthesis method of the compound represented by Chemical Formula 3-1-1A.

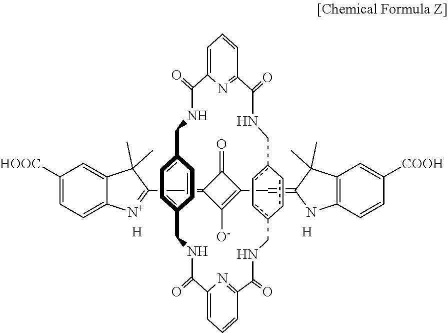

The compound represented by Chemical Formula Z had the following MALDI-TOF analysis result.

m/z calcd for C58H51N8010, (1019.37); found, 1019.37.

### Preparation of Green Pigment Dispersion

Green pigment dispersions of Preparation Examples 1 to 14 and Comparative Examples 1 to 4 were prepared to have each composition shown in Tables 1 to 4.

The green pigment dispersion was obtained by mixing a green pigment, a dispersant, a dispersion aid, and a solvent, adding 300 parts by weight of zirconia beads (diameter: 0.4 μm) based on 100 parts by weight of this mixture thereto, shaking and dispersing the mixture for 3 hours by using a paint shaker, and removing the zirconia beads therefrom by filtration.

TABLE 1
(unit: wt %)

| | | Preparation | Preparation | Preparation | Preparation | Preparation | Preparation |
|---|---|---|---|---|---|---|---|
| | | Example 1 | Example 2 | Example 3 | Example 4 | Example 5 | Example 6 |
| Green pigment | | 12.0 | 12.0 | 12.0 | 12.0 | 12.0 | 12.0 |
| Dispersant | | 2.5 | 2.5 | 2.5 | 2.5 | 2.5 | 2.5 |
| Dispersion | Synthesis | 0.5 | — | — | — | — | — |
| aid | Example 1 | | | | | | |
| | Synthesis | — | 0.5 | — | — | — | — |
| | Example 2 | | | | | | |
| | Synthesis | — | — | 0.5 | — | — | — |
| | Example 3 | | | | | | |
| | Synthesis | — | — | — | 0.5 | — | — |
| | Example 4 | | | | | | |
| | Synthesis | — | — | — | — | 0.5 | — |
| | Example 5 | | | | | | |
| | Synthesis | — | — | — | — | — | 0.5 |
| | Example 6 | | | | | | |

| | | | | | | |
|---|---|---|---|---|---|---|
| Solvent | 85.0 | 85.0 | 85.0 | 85.0 | 85.0 | 85.0 |

TABLE 2
(unit: wt %)

| | | Preparation | Preparation | Preparation |
|---|---|---|---|---|
| | | Example 7 | Example 8 | Example 9 |
| Green pigment | | 12.0 | 12.0 | 12.0 |
| Dispersant | | 2.5 | 2.5 | 2.5 |
| Dispersion | Synthesis | 0.5 | — | — |
| aid | Example 7 | | | |
| | Synthesis | — | 0.5 | — |
| | Example 8 | | | |
| | Synthesis | — | — | 0.5 |
| | Example 9 | | | |

| | | | |
|---|---|---|---|
| Solvent | 85.0 | 85.0 | 85.0 |

TABLE 3
(unit: wt %)

| | | | | Preparation | | |
|---|---|---|---|---|---|---|
| | | | | Example 12 | | |
| | | Preparation | Preparation | (=Preparation | Preparation | Preparation |
| | | Example 10 | Example 11 | Example 1) | Example 13 | Example 14 |
| Green pigment | | 12.0 | 12.0 | 12.0 | 12.0 | 12.0 |
| Dispersant | | 2.5 | 2.5 | 2.5 | 2.5 | 2.5 |
| Dispersion | Synthesis | 0.3 | 0.4 | 0.5 | 0.6 | 1.0 |
| aid | Example 1 | | | | | |

| | | | | | |
|---|---|---|---|---|---|
| Solvent | 85.2 | 85.1 | 85.0 | 84.9 | 84.5 |

TABLE 4
(unit: wt %)

| | | Comparative | Comparative | Comparative | Comparative |
|---|---|---|---|---|---|
| | | Preparation | Preparation | Preparation | Preparation |
| | | Example 1 | Example 2 | Example 3 | Example 4 |
| Green pigment | | 12.0 | 12.0 | 12.0 | 12.0 |
| Dispersant | | 2.5 | 2.5 | 2.5 | 2.5 |
| Dispersion | Comparative | — | 0.5 | — | — |
| aid | Synthesis | | | | |
| | Example 1 | | | | |
| | Comparative | — | — | 0.5 | — |
| | Synthesis | | | | |
| | Example 2 | | | | |
| | Comparative | — | — | — | 0.5 |
| | Synthesis | | | | |
| | Example 3 | | | | |

| | | | | |
|---|---|---|---|---|
| Solvent (PGMEA) | 85.0 | 85.0 | 85.0 | 85.0 |

The materials used in Tables 1 to 4 were as follows.

Green pigment: C.I. PIGMENT Green 58 G58

Dispersant: BYK-LPN6919 (Manufacturer: BYK)

Dispersion aid: each compound of Synthesis Examples 1 to 9, Comparative

### Synthesis Examples 1 to 3

Solvent: propylene glycol monomethylether acetate (PGMEA)

### Preparation Example 15: Preparation of Yellow Pigment Dispersion

A yellow pigment dispersion was prepared by mixing 12.0 parts by weight of a yellow pigment (C.I. PIGMENT Yellow 138), 3.0 parts by weight of a dispersant (BYK-LPN6919, Manufacturer: BYK), and 85.0 parts by weight of a solvent (propylene glycol monomethylether acetate, PGMEA), adding 300 parts by weight of zirconia beads (diameter: 0.4 μm) based on 100 parts by weight of this mixture thereto, shaking and dispersing the mixture by a paint shaker for 3 hours, and removing the zirconia beads by filtration.

### Preparation of Photosensitive Resin Composition

The photosensitive resin compositions of Examples 1 to 14 and Comparative Examples 1 to 4 were prepared by mixing the compositions shown in Tables 5 to 8.

Each photosensitive colored resin composition was prepared by mixing the green pigment dispersion, the yellow pigment dispersion, the photopolymerizable monomer, the photopolymerization initiator, the binder resin, and the solvent.

TABLE 5

| | | | Example 1 | Example 2 | Example 3 | Example 4 | Example 5 | Example 6 |
|---|---|---|---|---|---|---|---|---|
| (A) | A-1 | Preparation | 20.0 | — | — | — | — | — |
| Colorant | | Example 1 | | | | | | |
| | | Preparation | — | 20.0 | — | — | — | — |
| | | Example 2 | | | | | | |
| | | Preparation | — | — | 20.0 | — | — | — |
| | | Example 3 | | | | | | |
| | | Preparation | — | — | — | 20.0 | — | — |
| | | Example 4 | | | | | | |
| | | Preparation | — | — | — | — | 20.0 | — |
| | | Example 5 | | | | | | |
| | | Preparation | — | — | — | — | — | 20.0 |
| | | Example 6 | | | | | | |

| | | | | | | |
|---|---|---|---|---|---|---|
| A-2 | 15.0 | 15.0 | 15.0 | 15.0 | 15.0 | 15.0 |

| (B) | B-1 | 5.0 | 5.0 | 5.0 | 5.0 | 5.0 | 5.0 |
|---|---|---|---|---|---|---|---|
| Photopolymerization | | | | | | | |
| monomer | | | | | | | |
| (C) | C-1 | 1.0 | 1.0 | 1.0 | 1.0 | 1.0 | 1.0 |
| Photopolymerization | C-2 | 0.5 | 0.5 | 0.5 | 0.5 | 0.5 | 0.5 |
| initiator | | | | | | | |
| (D) Binder resin | | 3.5 | 3.5 | 3.5 | 3.5 | 3.5 | |
| (E) Solvent | E-1 | 56.0 | 56.0 | 56.0 | 56.0 | 56.0 | 56.0 |

TABLE 6

| | | | Example 7 | Example 8 | Example 9 | Example 10 | Example 11 |
|---|---|---|---|---|---|---|---|
| (A) | A-1 | Preparation | 20.0 | — | — | — | — |
| Colorant | | Example 7 | | | | | |
| | | Preparation | — | 20.0 | — | — | — |
| | | Example 8 | | | | | |
| | | Preparation | — | — | 20.0 | — | — |
| | | Example 9 | | | | | |

| | | | | | |
|---|---|---|---|---|---|
| A-2 | 15.0 | 15.0 | 15.0 | 15.0 | 15.0 |

| (B) Photopolymerization monomer | | 5.0 | 5.0 | 5.0 | 5.0 | 5.0 |
|---|---|---|---|---|---|---|
| (C) | C-1 | 1.0 | 1.0 | 1.0 | 1.0 | 1.0 |
| Photopolymerization | C-2 | 0.5 | 0.5 | 0.5 | 0.5 | 0.5 |
| initiator | | | | | | |

| | | | | | |
|---|---|---|---|---|---|
| (D) Binder resin | 3.5 | 3.5 | 3.5 | 3.5 | 3.5 |
| (E) Solvent | 56.0 | 56.0 | 56.0 | 56.0 | 56.0 |

TABLE 7

| | | | | | Example 12 | | |
|---|---|---|---|---|---|---|---|
| | | | Example 10 | Example 11 | (=Example 1) | Example 13 | Example 14 |
| (A) | A-1 | Preparation | 20.0 | — | — | — | — |
| Colorant | | Example 12 | | | | | |
| | | Preparation | — | 20.0 | — | — | — |
| | | Example 13 | | | | | |
| | | Preparation | — | — | 20.0 | — | — |
| | | Example 14 | | | | | |
| | | Preparation | — | — | — | 20.0 | — |
| | | Example 15 | | | | | |
| | | Preparation | — | — | — | — | 20.0 |
| | | Example 16 | | | | | |

| | | | | | |
|---|---|---|---|---|---|
| A-2 | 15.0 | 15.0 | 15.0 | 15.0 | 15.0 |

| (B) Photopolymerization monomer | | 5.0 | 5.0 | 5.0 | 5.0 | 5.0 |
|---|---|---|---|---|---|---|
| (C) | C-1 | 1.0 | 1.0 | 1.0 | 1.0 | 1.0 |
| Photopolymerization | C-2 | 0.5 | 0.5 | 0.5 | 0.5 | 0.5 |
| initiator | | | | | | |

| | | | | | |
|---|---|---|---|---|---|
| (D) Binder resin | 3.5 | 3.5 | 3.5 | 3.5 | 3.5 |
| (E) Solvent | 56.0 | 56.0 | 56.0 | 56.0 | 56.0 |

TABLE 8

| | | | Comparative | Comparative | Comparative | Comparative |
|---|---|---|---|---|---|---|
| | | | Example 1 | Example 2 | Example 3 | Example 4 |
| (A) | A-1 | Comparative | 20.0 | — | — | — |
| Colorant | | Preparation | | | | |
| | | Example 1 | | | | |
| | | Comparative | — | 20.0 | — | — |
| | | Preparation | | | | |
| | | Example 2 | | | | |
| | | Comparative | — | — | 20.0 | — |
| | | Preparation | | | | |
| | | Example 3 | | | | |
| | | Comparative | — | — | — | 20.0 |
| | | Preparation | | | | |
| | | Example 4 | | | | |

| | | | | |
|---|---|---|---|---|
| A-2 | 15.0 | 15.0 | 15.0 | 15.0 |

| (B) Photopolymerization monomer | | 5.0 | 5.0 | 5.0 | 5.0 |
|---|---|---|---|---|---|
| (C) | C-1 | 1.0 | 1.0 | 1.0 | 1.0 |
| Photopolymerization | C-2 | 0.5 | 0.5 | 0.5 | 0.5 |
| initiator | | | | | |

| | | | | |
|---|---|---|---|---|
| (D) Binder resin | 3.5 | 3.5 | 3.5 | |
| (E) Solvent | 56.0 | 56.0 | 56.0 | 56.0 |

The materials used in Tables 5 to 8 are as follows.
(A) Colorant
(A-1) Each green pigment dispersion of Preparation Examples 1 to 10 and Comparative Preparation Examples 1 to 3
(A-2) Yellow pigment dispersion of Preparation Example 15
(B) Photopolymerizable Monomer
Dipentaerythritol hexaacrylate (DPHA, manufacturer: Nippon Kayaku Co., Ltd.)
(C) Photopolymerization initiator
(C-1) 1,2-octanedione
(C-2) 2-dimethylamino-2-(4-methyl-benzyl)-1-(4-morpholin-4-yl-phenyl)-butan-1-one
(D) Binder resin
Resin copolymerized with benzyl methacrylate and methacrylic acid at 85:15 (Mw=22,000 g/mol)
(E) Solvent
propylene glycol monomethylether acetate (PGMEA)

### Evaluation Example 1: Dispersibility and Dispersion Stability

Each of the photosensitive resin compositions according to Examples 1 to 14 and Comparative Examples 1 to 4 was measured a solid particle diameter by using dynamic light scattering analysis equipment, and the results are shown in Table 9.

In addition, the photosensitive resin compositions according to Examples 1 to 14 and Comparative Examples 1 to 4 were evaluated with respect to viscosity before and after stored at 23° C. for 1 week at 5 rpm (rpm with a Torque value of 50% to 100%) by using Brookfield DV-II Pro viscometer and CPE-52 Spindle at 25° C., and the results are shown in Table 9.

TABLE 9

| | Solid particle diameter (nm) | | | Viscosity (cPs) | | |
|---|---|---|---|---|---|---|
| | After | After | | After | After | |
| | preparation | 1 week | Difference | preparation | 1 week | Difference |
| Example 1 | 47 | 46 | 1 | 4.56 | 4.47 | 0.09 |
| Example 2 | 45 | 44 | 1 | 4.47 | 4.55 | 0.08 |
| Example 3 | 46 | 46 | 1 | 4.64 | 4.57 | 0.07 |
| Example 4 | 45 | 44 | 1 | 5.19 | 5.04 | 0.15 |
| Example 5 | 44 | 44 | 0 | 4.44 | 4.58 | 0.14 |
| Example 6 | 44 | 44 | 0 | 4.64 | 4.78 | 0.14 |
| Example 7 | 46 | 45 | 1 | 4.15 | 4.34 | 0.19 |
| Example 8 | 48 | 48 | 0 | 4.54 | 4.71 | 0.17 |
| Example 9 | 49 | 46 | 3 | 4.46 | 4.68 | 0.22 |
| Example 10 | 44 | 44 | 0 | 4.30 | 4.42 | 0.18 |
| Example 11 | 45 | 46 | 1 | 4.40 | 4.55 | 0.15 |
| Example 12 | 47 | 46 | 1 | 4.56 | 4.47 | 0.09 |
| (=Example 1) | | | | | | |
| Example 13 | 47 | 45 | 2 | 4.11 | 4.19 | 0.08 |
| Example 14 | 47 | 49 | 2 | 5.08 | 5.11 | 0.03 |
| Comparative | 50 | 64 | 14 | 5.14 | 5.88 | 0.74 |
| Example 1 | | | | | | |
| Comparative | 50 | 55 | 5 | 4.57 | 6.02 | 1.45 |
| Example 2 | | | | | | |
| Comparative | 49 | 59 | 10 | 5.18 | 6.11 | 0.93 |
| Example 3 | | | | | | |
| Comparative | 47 | 54 | 7 | 4.97 | 6.22 | 1.25 |
| Example 4 | | | | | | |

* In Table 9, when a change in a solid particle diameter and viscosity was 10%, “satisfactory” was given

Referring to Table 9, the photosensitive resin compositions according to Examples 1 to 14 exhibited a smaller change in the viscosity as well as in the solid particle diameter before and after stored for 1 week than the photosensitive resin compositions according to Comparative Examples 1 to 4.

### Evaluation Example 2: Coloring Power and Contrast

Each photosensitive resin composition of Examples 1 to 14 and Comparative Examples 1 to 4 was coated to be 0.6 μm thick on a 1 mm-thick glass substrate, which was degreased and washed, and then, dried on a hot plate at 90° C. for 2 minutes to obtain a film. Subsequently, the film was exposed to light by using a high-pressure mercury lamp with a main wavelength of 365 nm and dried in a forced convection drying furnace at 200° C. for 5 minutes to obtain a color filter specimen.

A pixel layer was evaluated were measuring a color coordinate (x, y), luminance (Y), and a thickness (THICK) of the color filter specimens by using a spectrophotometer (MCPD3000, Otsuka Electronics Co., Ltd.), and the results are shown in Tables 8 to 10.

TABLE 10

| | | | | THICK |
|---|---|---|---|---|
| | x | y | Y | (μm) |
| Example 1 | 348 | 0.574 | 72.8 | 6,131 |
| Example 2 | 348 | 0.573 | 73.2 | 6,078 |
| Example 3 | 350 | 0.569 | 75.2 | 6,114 |
| Example 4 | 348 | 0.572 | 73.1 | 6,002 |
| Example 5 | 348 | 0.571 | 72.7 | 6,099 |
| Example 6 | 348 | 0.572 | 72.5 | 6,108 |
| Example 7 | 348 | 0.574 | 72.8 | 6,004 |
| Example 8 | 348 | 0.573 | 73.1 | 6,001 |
| Example 9 | 348 | 0.572 | 73.3 | 6,011 |
| Example 10 | 348 | 0.572 | 73.2 | 6,022 |
| Example 11 | 348 | 0.574 | 72.9 | 6,100 |
| Example 12 | 348 | 0.574 | 72.8 | 6,131 |
| (=Example 1) | | | | |
| Example 13 | 348 | 0.575 | 73.1 | 6,004 |
| Example 14 | 348 | 0.576 | 73.1 | 6,011 |
| Comparative | 351 | 0.556 | 72.3 | 6,899 |
| Example 1 | | | | |
| Comparative | 352 | 0.554 | 72.0 | 6,771 |
| Example 2 | | | | |
| Comparative | 352 | 0.552 | 71.5 | 6,727 |
| Example 3 | | | | |
| Comparative | 352 | 0.555 | 71.5 | 6,687 |
| Example 4 | | | | |

* In Table 10, the higher Y and the thinner, the more excellent characteristics.

Referring to Table 10, the color filter specimens of Examples 1 to 14, compared to the color filter specimens of Comparative Examples 1 and 4, exhibited improved coloring power and luminance.

By using the dispersion aid represented by Chemical Formula 1, it is possible to provide a photosensitive resin composition that has high dispersibility and dispersion stability and has excellent coloring power and luminance even when the color filter is thinned.

By way of summation and review, in color filters manufactured using a pigment dispersion method, not only excellent pattern characteristics but also luminance characteristics along with high color reproducibility are required.

One or more embodiments may provide a photosensitive resin composition that has high dispersibility and dispersion stability and has excellent coloring power and luminance even when the color filter is thinned.

Accordingly, excellent color filters and display devices may be implemented by using the photosensitive resin composition according to some example embodiments.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated.

Accordingly, it will be understood by those of skill in the art that various changes in form and details may be made without departing from the spirit and scope of the present invention as set forth in the following claims.

What is claimed is:

1. A photosensitive resin composition, comprising:
a colorant;
a photopolymerizable compound;
a photopolymerization initiator;
a binder resin; and
a solvent;
wherein:
the colorant includes a pigment, a dispersant, and a dispersion aid represented by Chemical Formula 1: 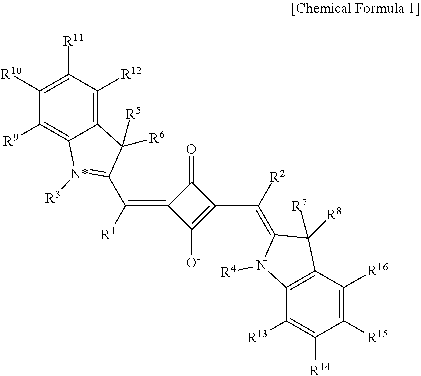
in Chemical Formula 1,
R¹ to R⁸ are each independently a hydrogen atom or a substituted or unsubstituted C1 to C20 alkyl group; and
R⁹ to R¹⁶ are each independently a hydrogen atom, a substituted or unsubstituted C1 to C20 alkyl group, or a substituted or unsubstituted acidic functional group, provided that at least one of R⁹ to R¹⁶ is an acidic functional group.

2. The photosensitive resin composition as claimed in claim 1, wherein both R¹ and R² are hydrogen atoms.

3. The photosensitive resin composition as claimed in claim 1, wherein both R³ and R⁴ are hydrogen atoms.

4. The photosensitive resin composition as claimed in claim 1, wherein all of R⁵ to R⁸ are each a substituted or unsubstituted methyl group.

5. The photosensitive resin composition as claimed in claim 1, wherein one of R⁹ to R¹² is a substituted or unsubstituted acidic functional group and one of R¹³ and R¹⁶ is a substituted or unsubstituted acidic functional group.

6. The photosensitive resin composition as claimed in claim 5, wherein:
the dispersion aid is represented by Chemical Formula 1-1: 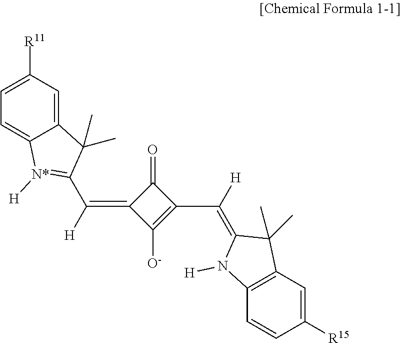
in R¹¹ and R¹⁵ are each independently a substituted or unsubstituted acidic functional group.

7. The photosensitive resin composition as claimed in claim 6, wherein the substituted or unsubstituted acidic functional group is an amine group having *—COOH or *—SO₃H at a terminal end.

8. The photosensitive resin composition as claimed in claim 7, wherein:
the substituted or unsubstituted acidic functional group is represented by Chemical Formula 1-1: 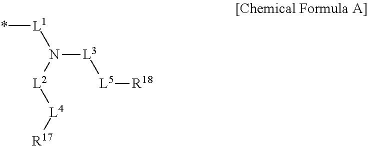
in Chemical Formula A,
L¹ is a single bond or a substituted or unsubstituted C1 to C20 alkylene group;
L² and L³ are a single bond, a substituted or unsubstituted C1 to C20 alkylene group, or *—(C═O)—*;
L⁴ and L⁵ are a single bond or a substituted or unsubstituted C1 to C20 alkylene group; and
R¹⁷ and R¹⁸ are each independently a hydrogen atom, *—COOH, or *—SO₃H, provided that at least one of R¹⁷ and R¹⁸ is *—COOH, or *—SO₃H.

9. The photosensitive resin composition as claimed in claim 8, wherein the dispersion aid includes a compound represented by one of the following Chemical Formulae:

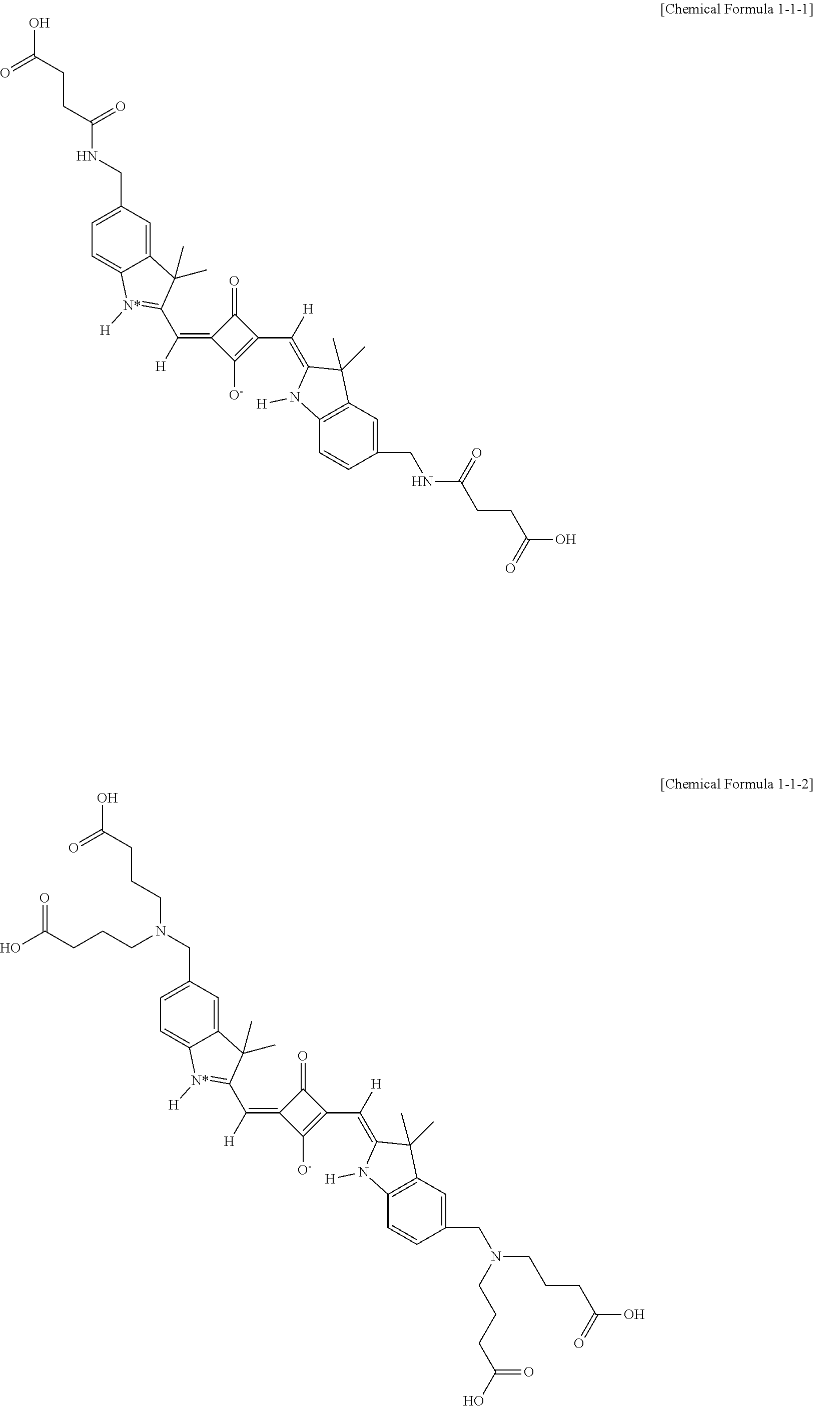

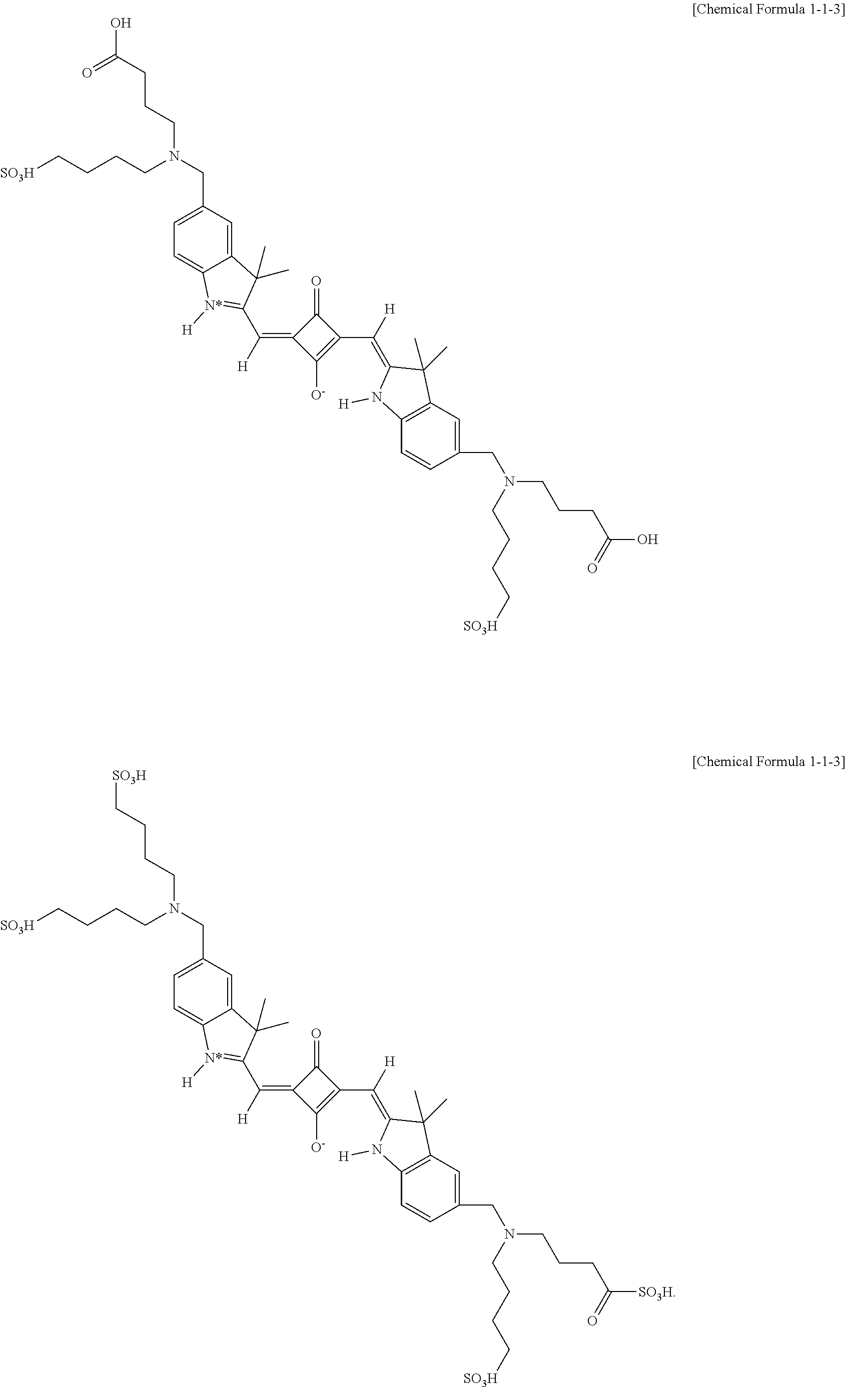

10. The photosensitive resin composition as claimed in claim 1, wherein:
the dispersion aid includes:
a compound represented by Chemical Formula 1 as a core, and
a shell surrounding the core, the shell being represented by Chemical Formula 2: 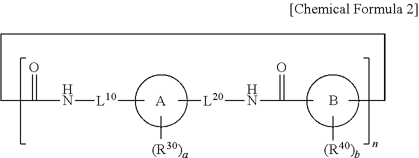
in Chemical Formula 2,
A and B are each independently a benzene ring, a pyridine ring, or a furan ring;
L¹⁰ and L²⁰ are each independently a single bond or a substituted or unsubstituted C1 to C10 alkylene group;
R³⁰ and R⁴⁰ are each independently a halogen;
a and b are each independently an integer of 0 to 4; and
n is an integer of 2 to 10.

11. The photosensitive resin composition as claimed in claim 10, wherein the shell is a compound represented by one of the following Chemical Formulae:

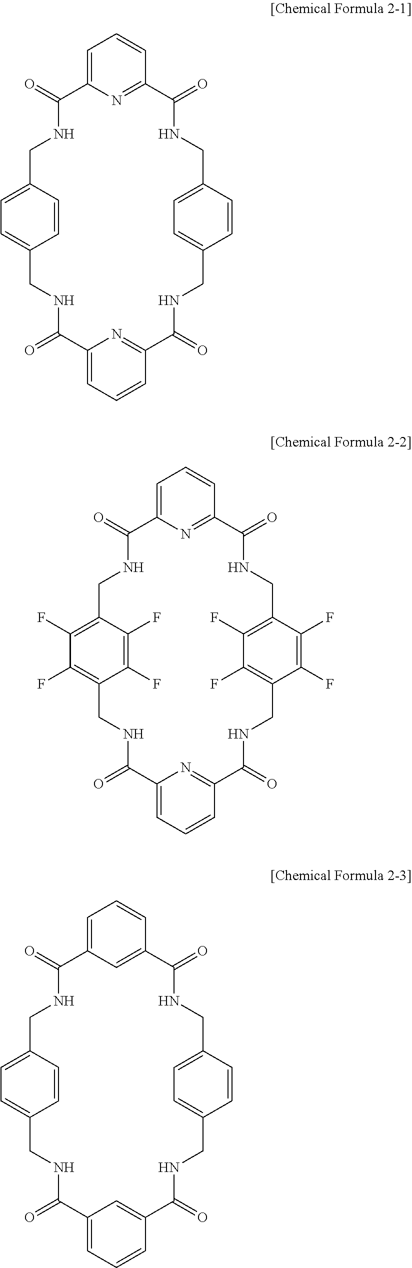 [SMILES: O=C1NCc2ccc(cc2)CNC(=O)c2cccc(n2)C(=O)NCc2ccc(cc2)CNC(=O)c2cccc1n2 ; O=C1NCc2c(F)c(F)c(c(F)c2F)CNC(=O)c2cccc(n2)C(=O)NCc2c(F)c(F)c(c(F)c2F)CNC(=O)c2cccc1n2 ; O=C1NCc2ccc(cc2)CNC(=O)c2cccc(c2)C(=O)NCc2ccc(cc2)CNC(=O)c2cccc1c2]

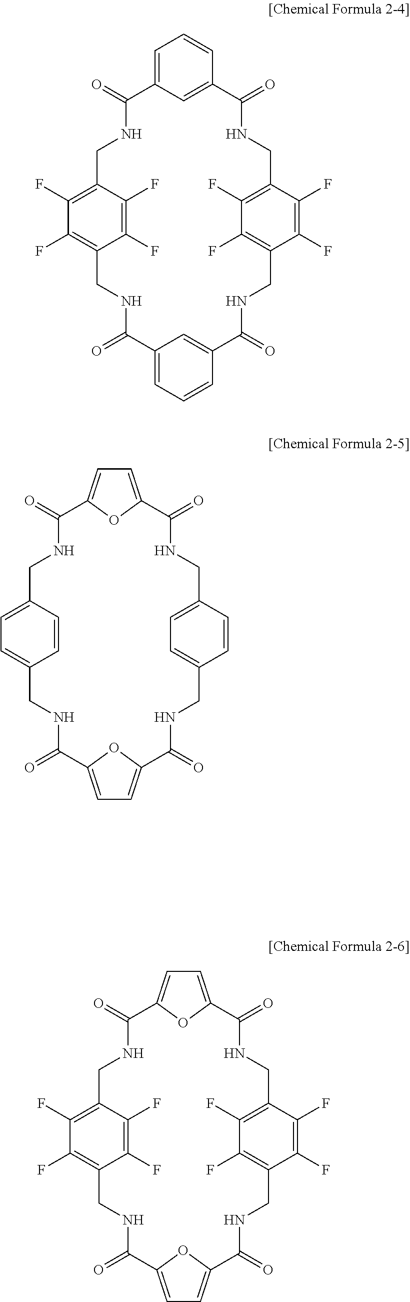 [SMILES: O=C1NCc2c(F)c(F)c(c(F)c2F)CNC(=O)c2cccc(c2)C(=O)NCc2c(F)c(F)c(c(F)c2F)CNC(=O)c2cccc1c2 ; O=C1NCc2ccc(cc2)CNC(=O)c2ccc(o2)C(=O)NCc2ccc(cc2)CNC(=O)c2ccc1o2 ; O=C1NCc2c(F)c(F)c(c(F)c2F)CNC(=O)c2ccc(o2)C(=O)NCc2c(F)c(F)c(c(F)c2F)CNC(=O)c2ccc1o2]

12. The photosensitive resin composition as claimed in claim 10, wherein the dispersion aid is represented by one of the following Chemical Formulae:

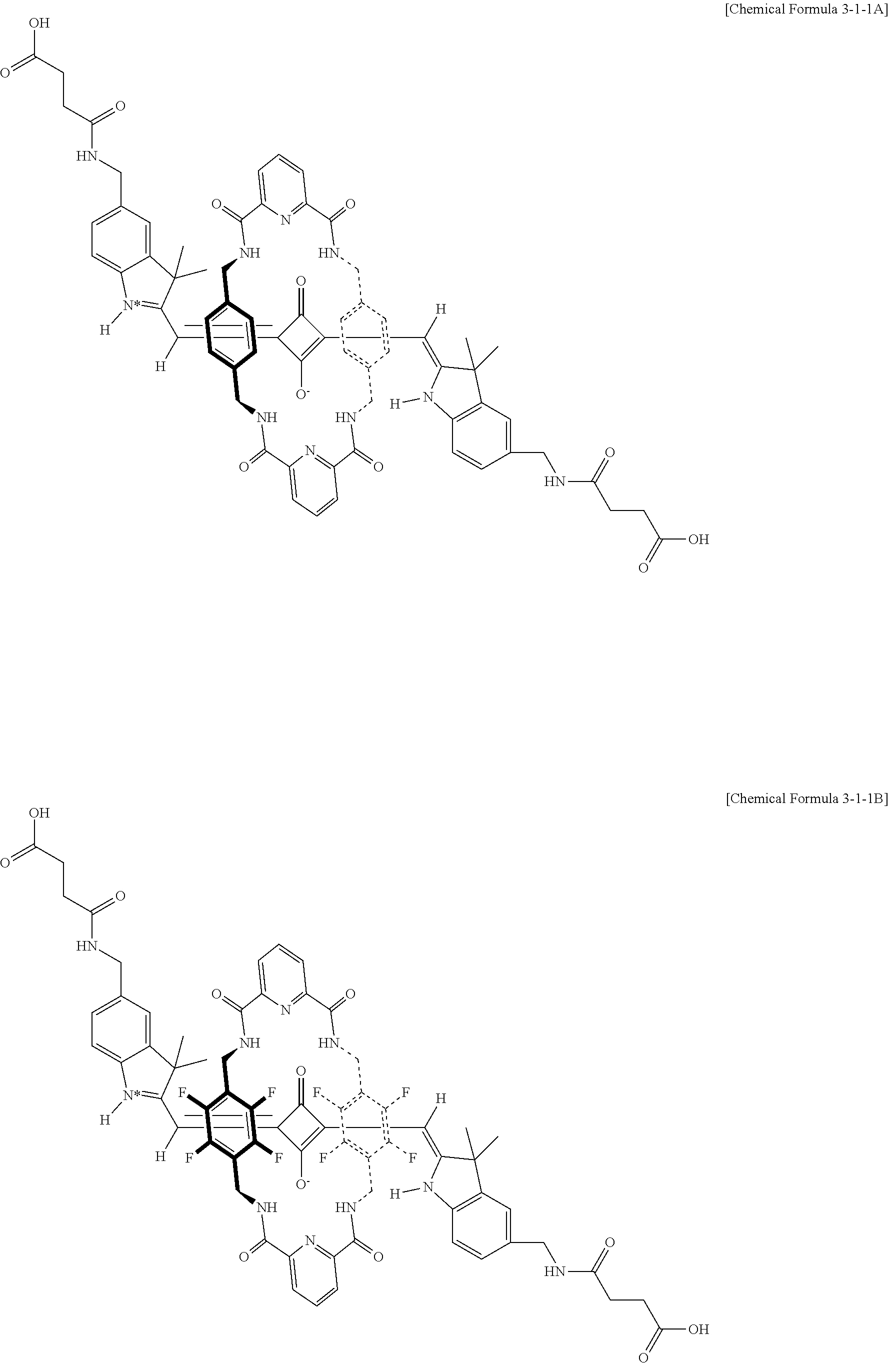

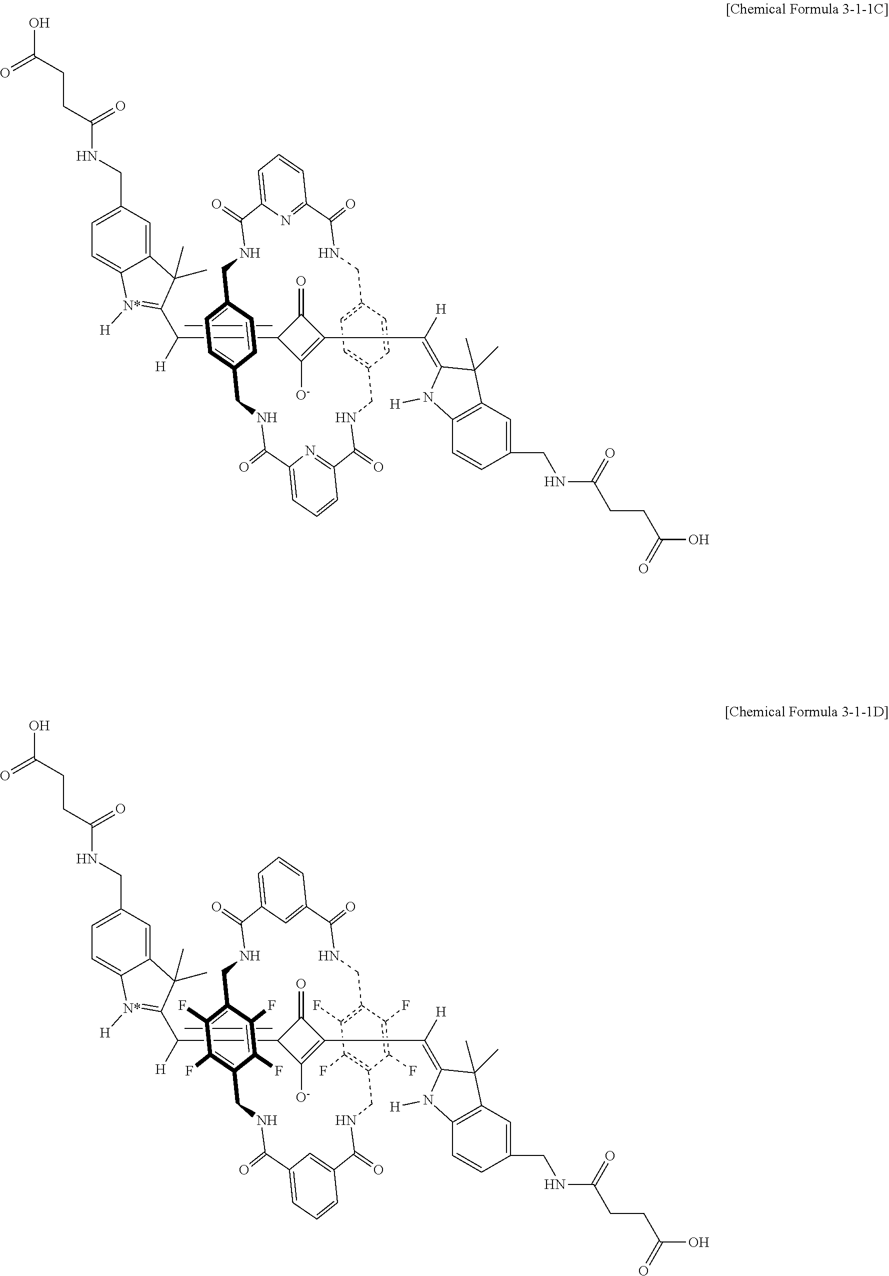

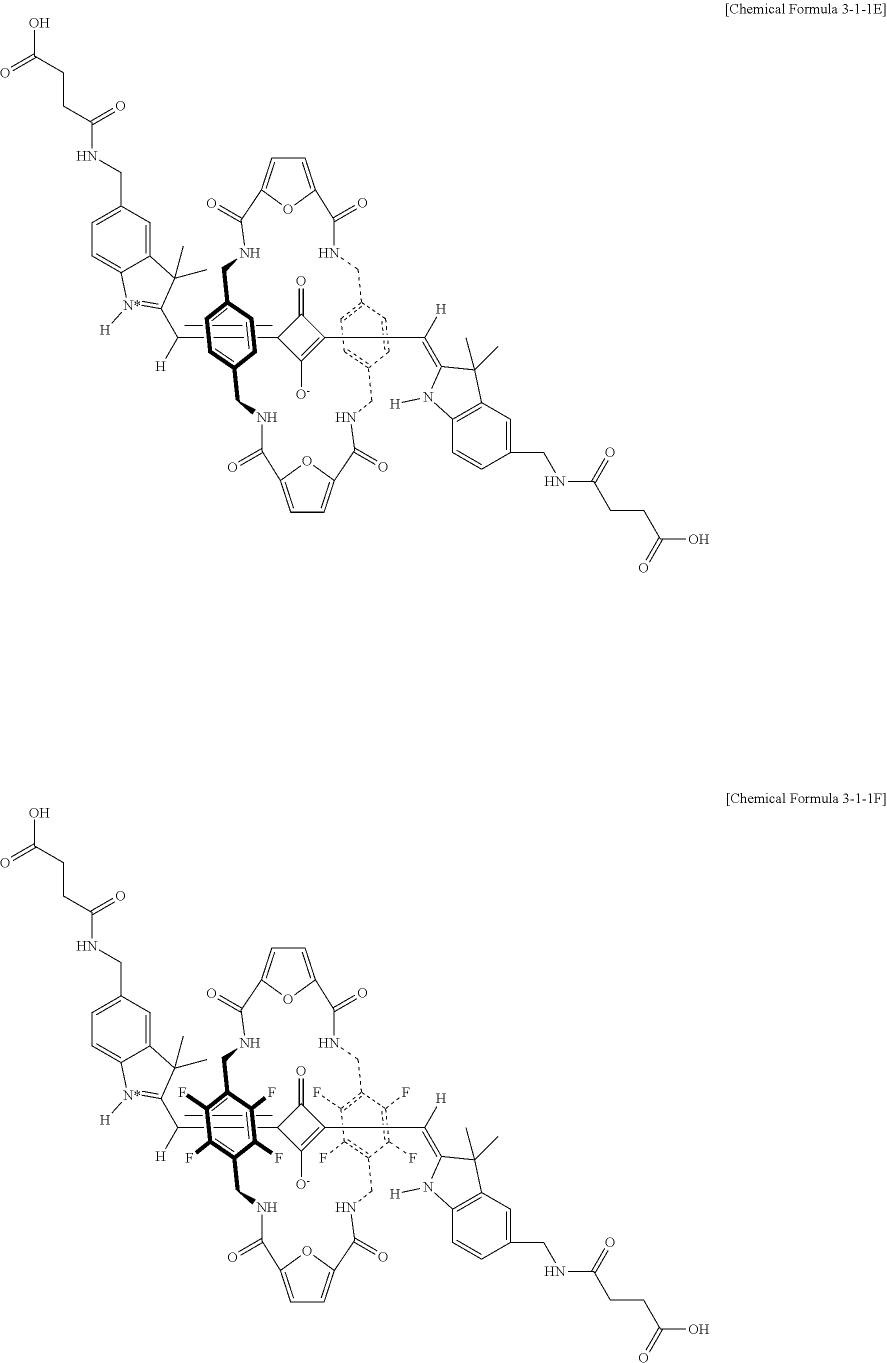

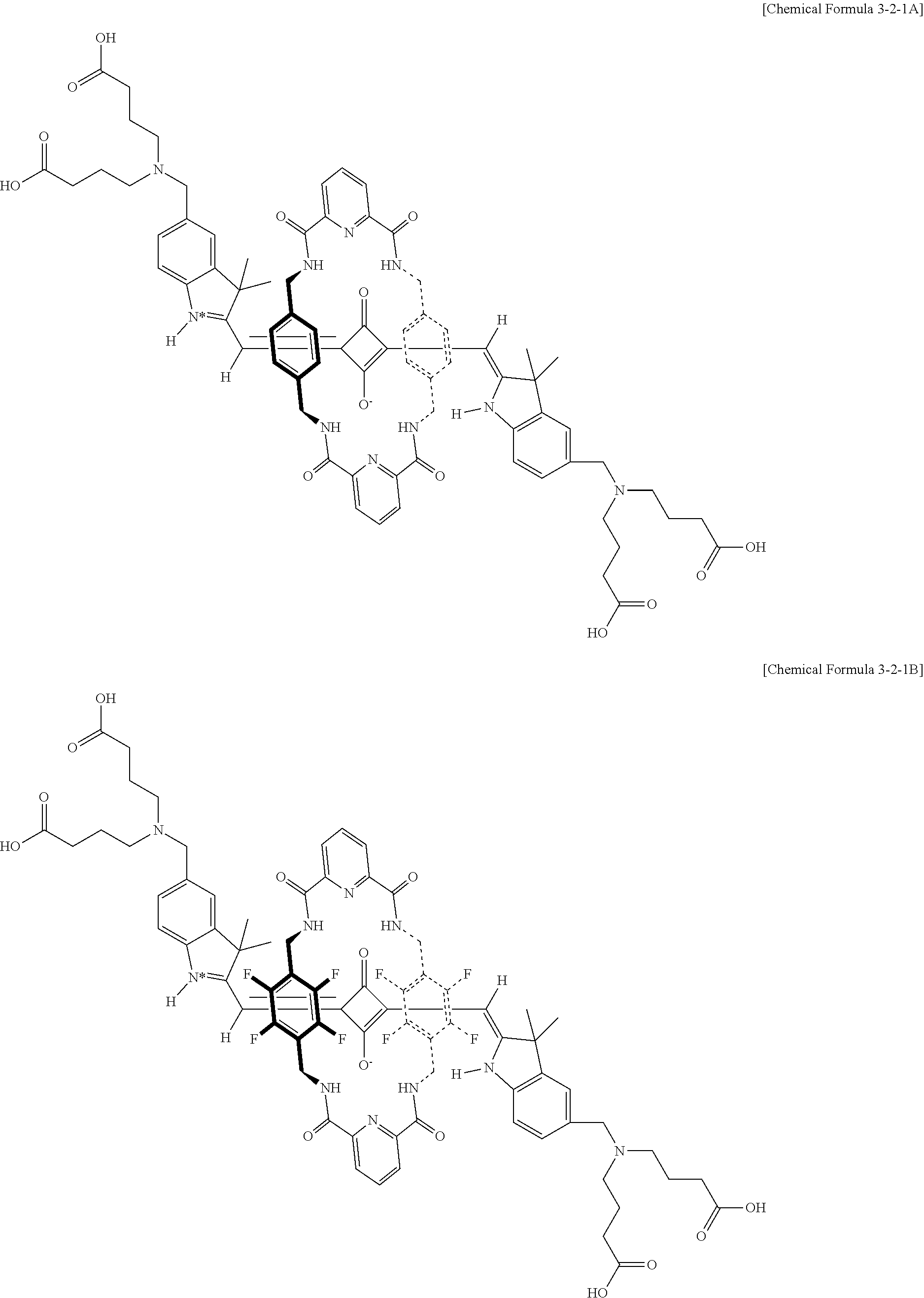

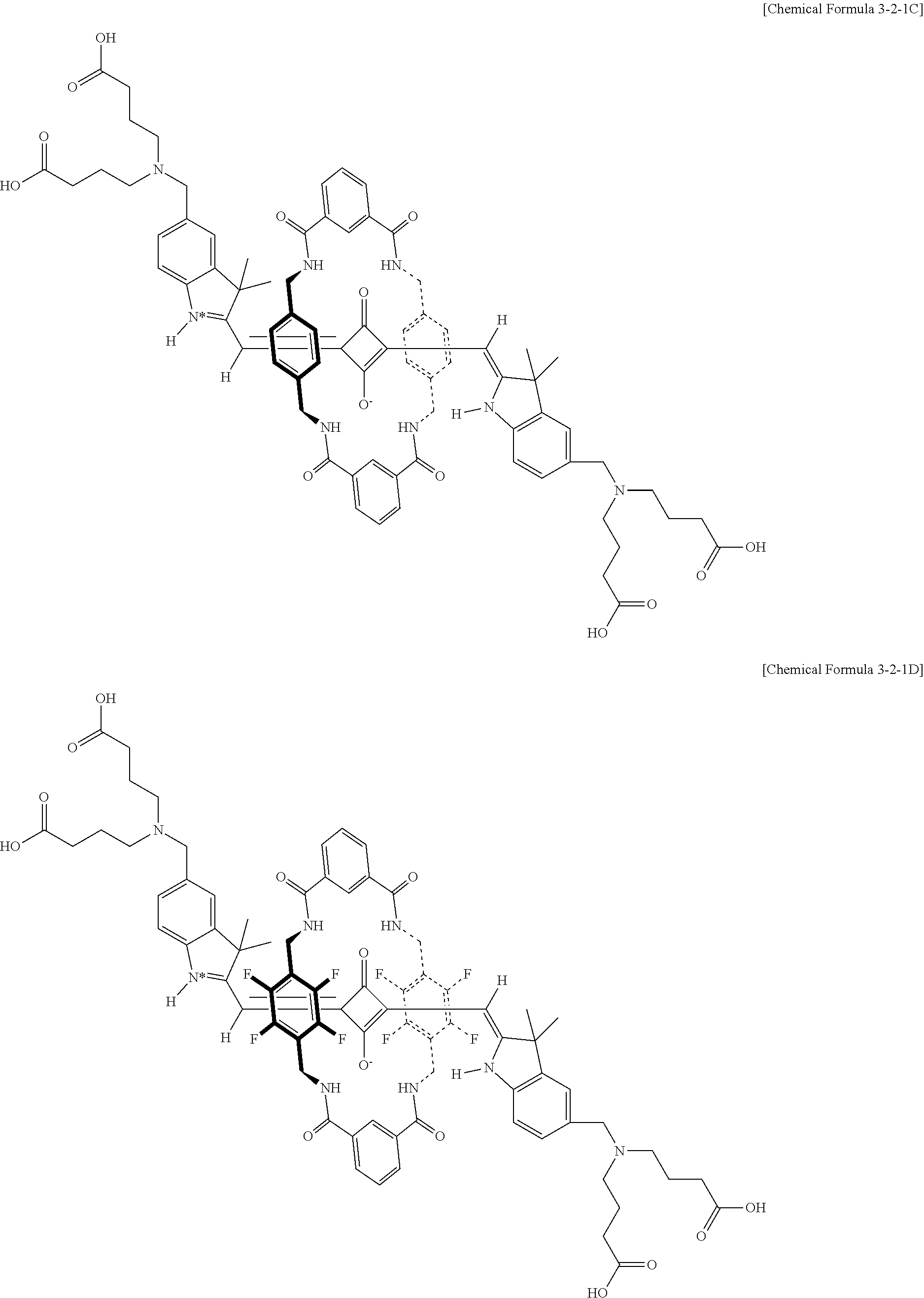

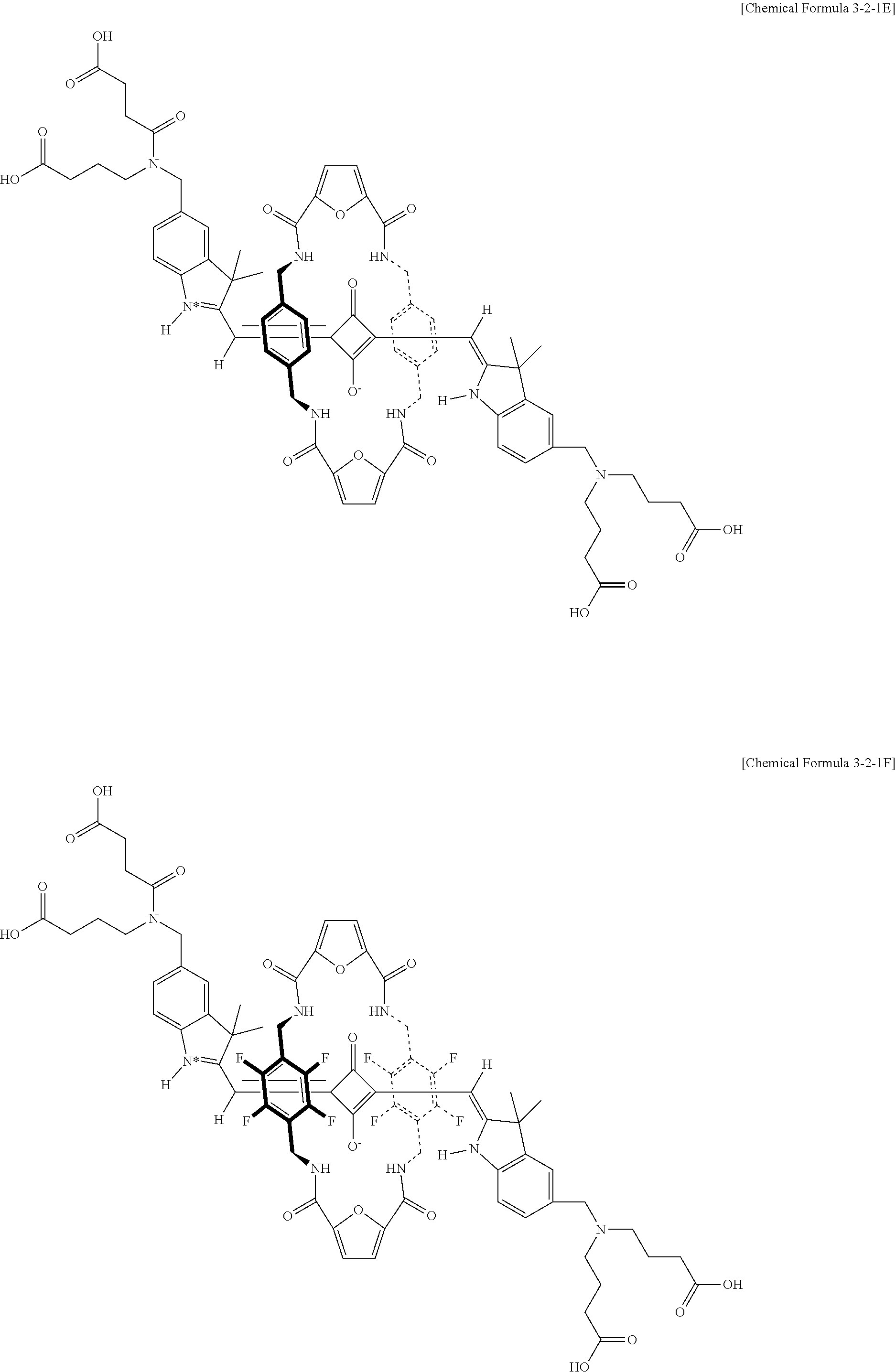

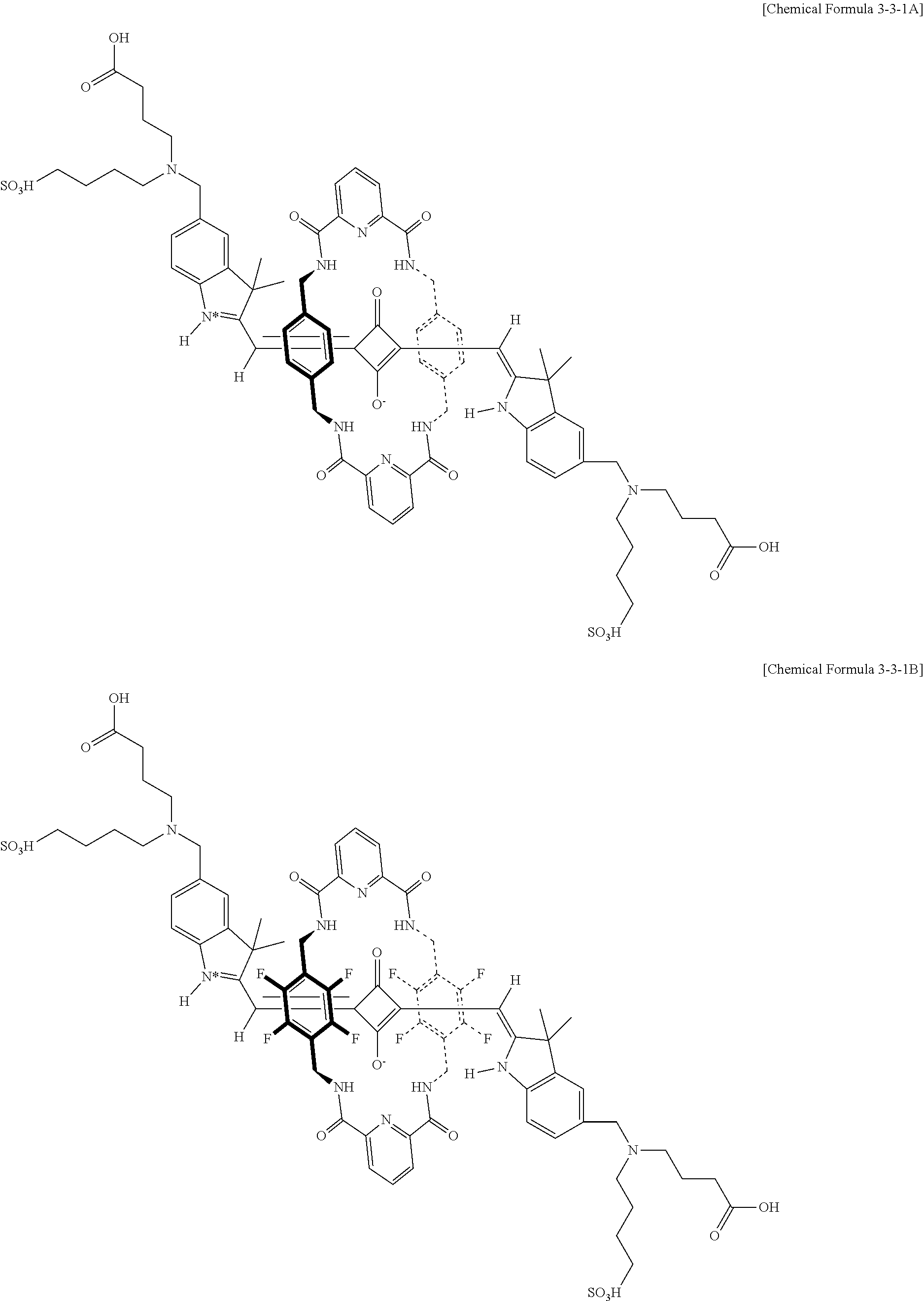

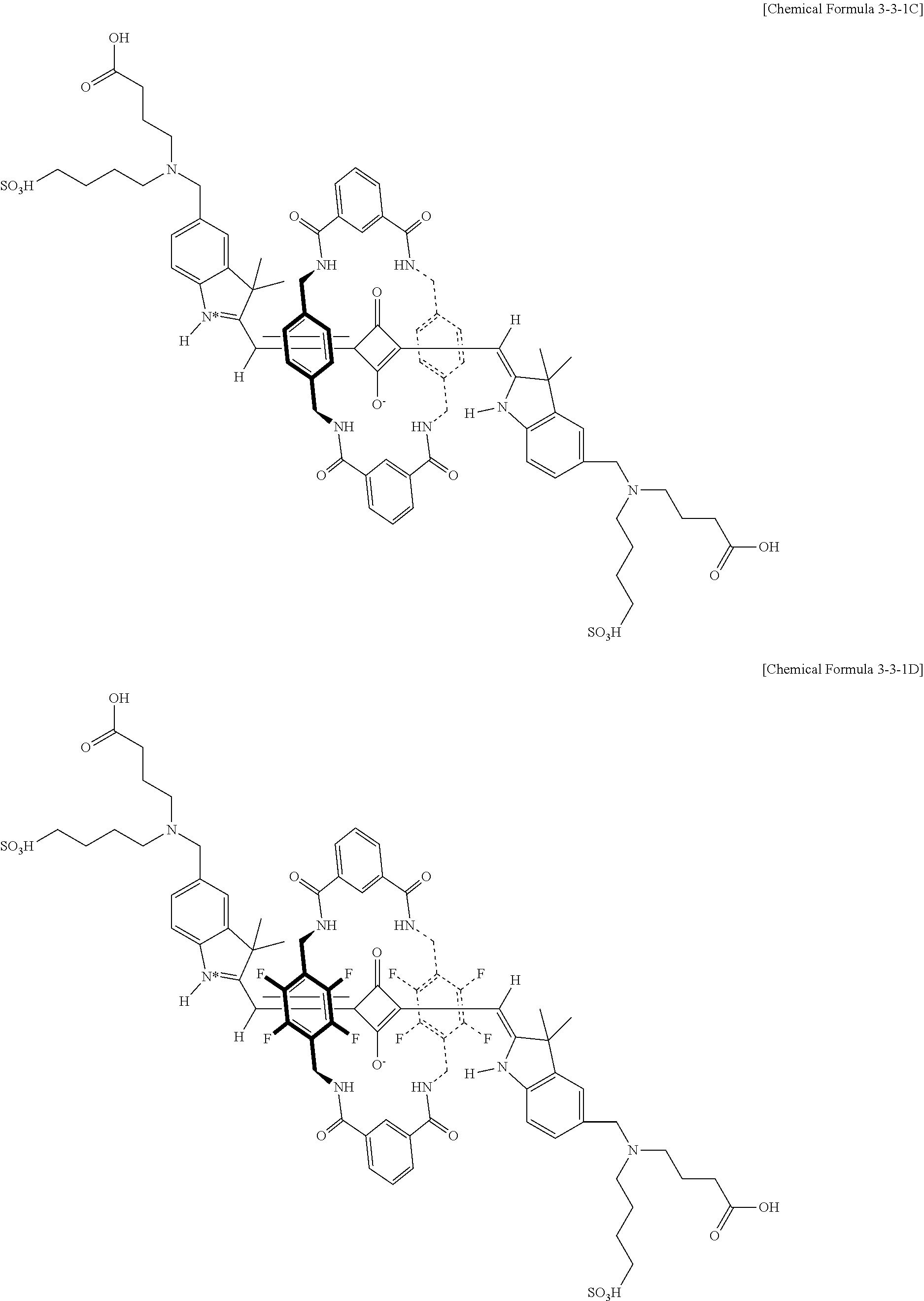

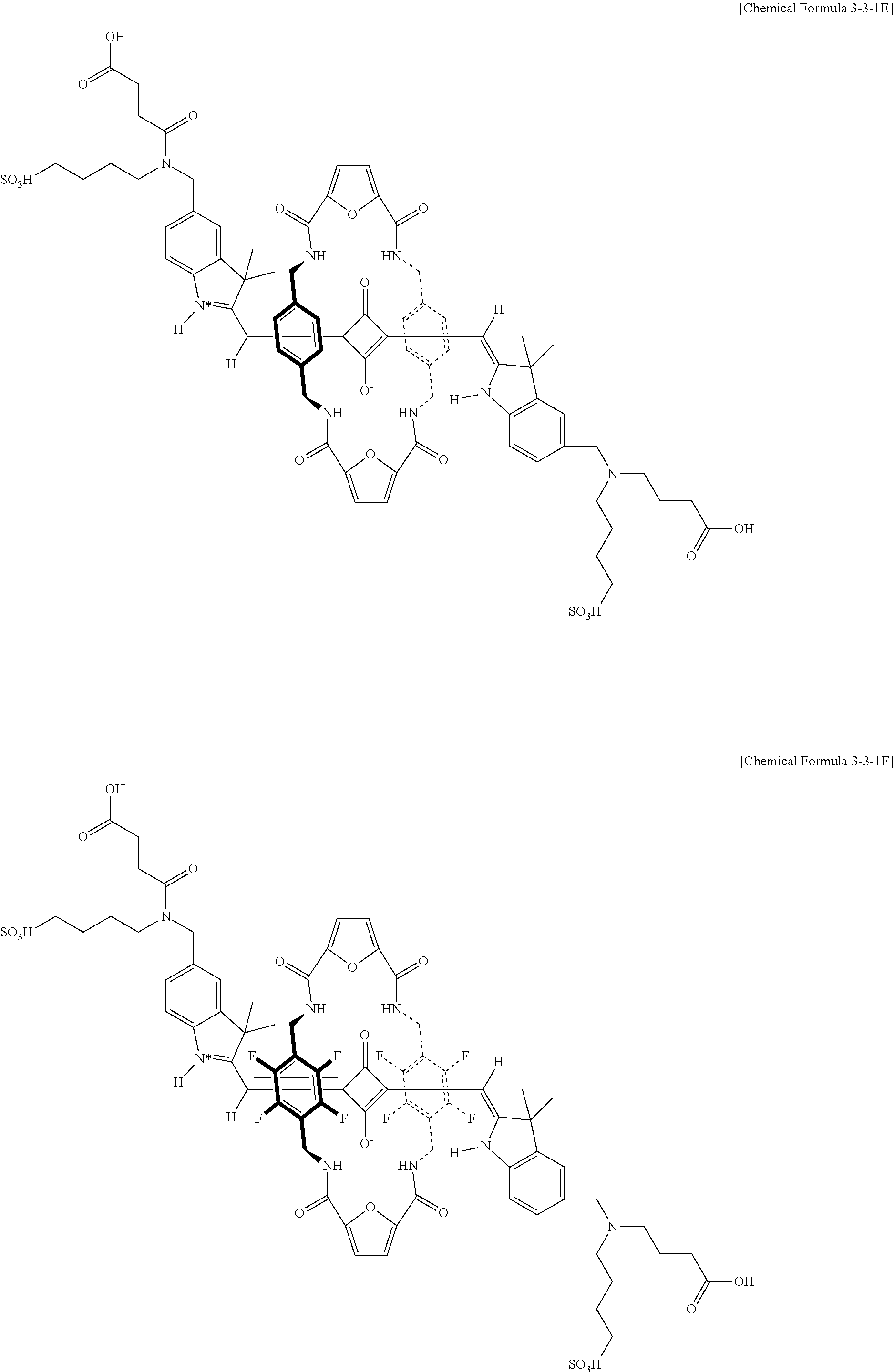

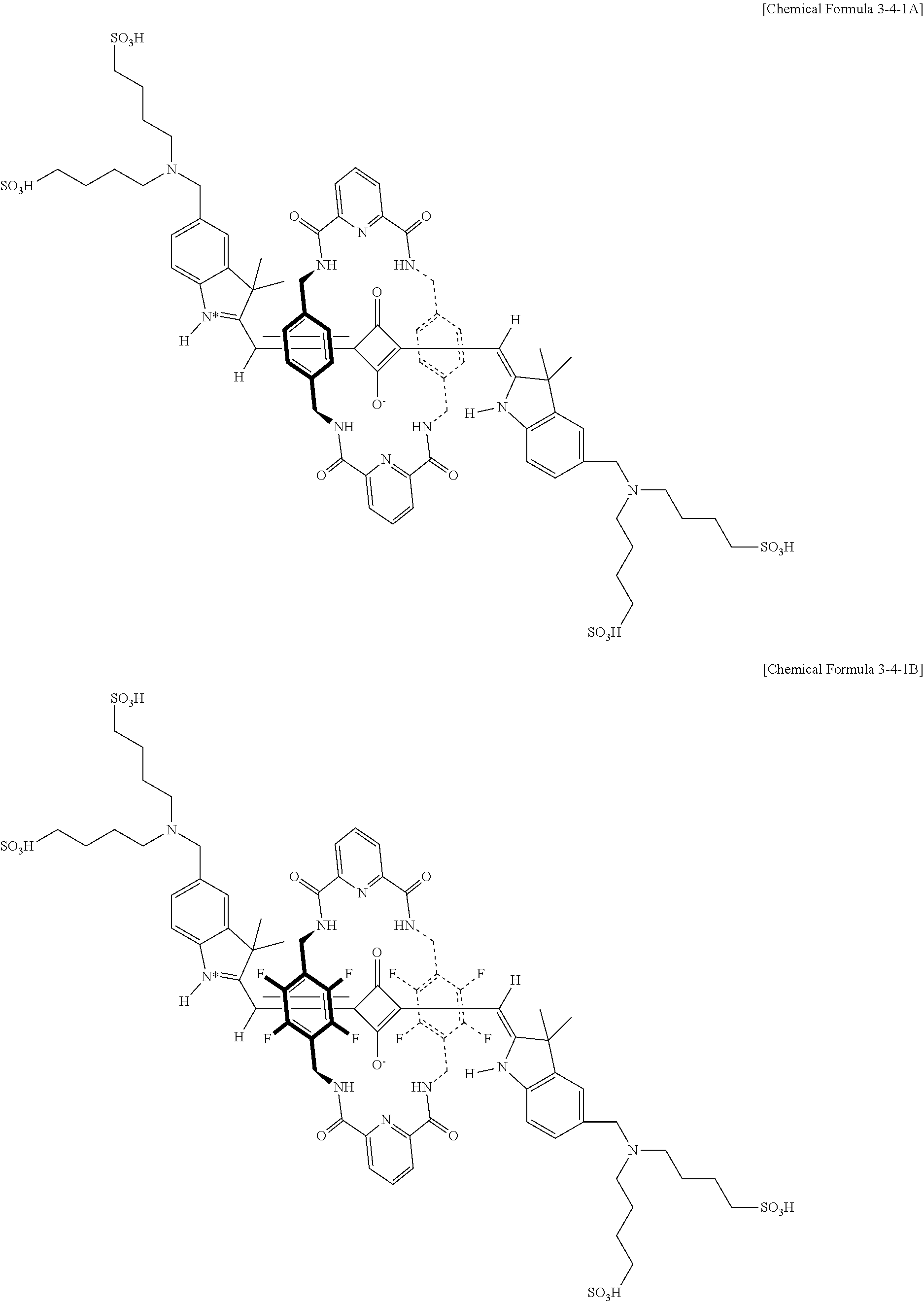

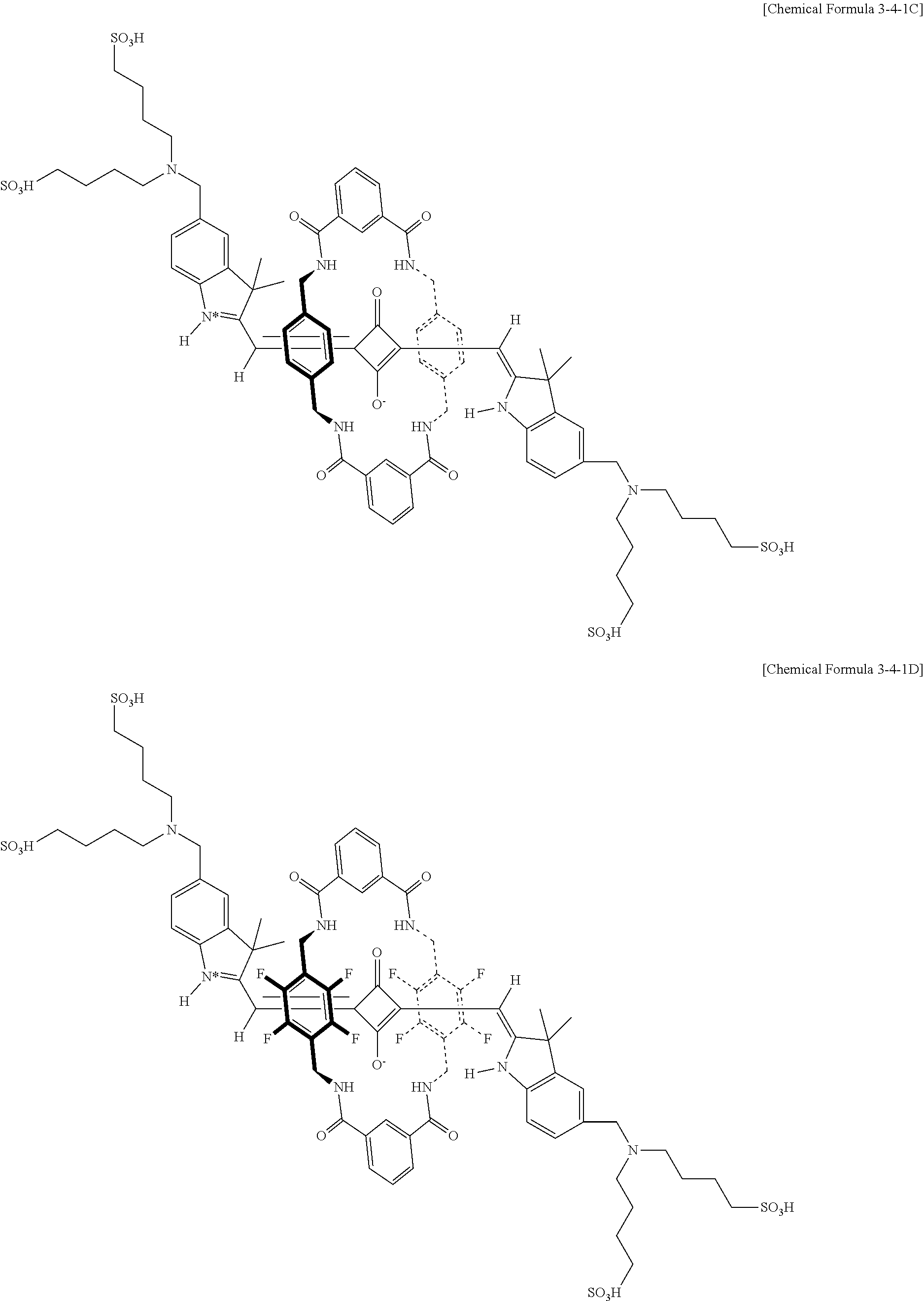

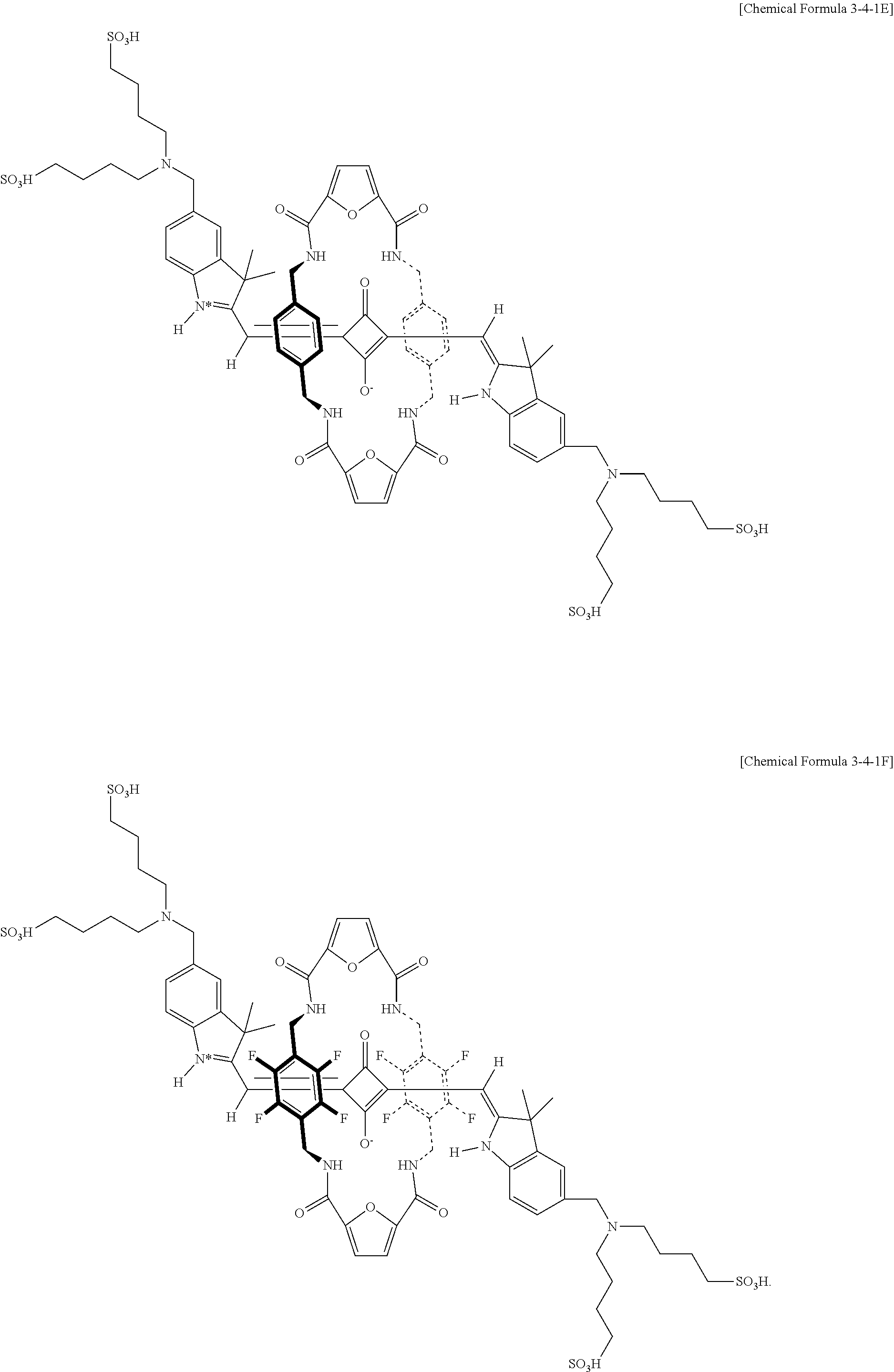

13. The photosensitive resin composition as claimed in claim 1, wherein the dispersion aid has a maximum absorption wavelength of about 530 nm to about 700 nm.

14. The photosensitive resin composition as claimed in claim 1, wherein the dispersion aid is included in an amount of about 0.5 wt % to about 6 wt %, based on a total weight of the photosensitive resin composition.

15. The photosensitive resin composition as claimed in claim 1, wherein a weight ratio of the dispersion aid and the pigment is about 1:20 to about 1:70.

16. The photosensitive resin composition as claimed in claim 1, wherein the photosensitive resin composition includes:
about 5 wt % to about 50 wt % of the colorant;
about 0.1 wt % to about 10 wt % of the photopolymerizable compound;
about 0.1 wt % to about 10 wt % of the photopolymerization initiator;
about 0.5 wt % to about 15 wt % of the binder resin; and
the solvent, all wt % being based on a total weight of the photosensitive resin composition.

17. A photosensitive resin layer manufactured using the photosensitive resin composition according to claim 1.

18. The photosensitive resin layer as claimed in claim 17, wherein the photosensitive resin layer is a color negative photoresist.

19. A color filter comprising the photosensitive resin layer according to claim 17.

20. A display device comprising the color filter according to claim 19.
